(19) United States
(12) Patent Application Publication
(10) Pub. No.: US 2024/0229107 A1
(43) Pub. Date: Jul. 11, 2024

(54) MULTI-PART OLIGONUCLEOTIDE PROBES AND METHODS OF USE THEREOF

(71) Applicant: Singular Genomics Systems, Inc., San Diego, CA (US)

(72) Inventor: Michael Lawson, Pasadena, CA (US)

(21) Appl. No.: 18/390,804

(22) Filed: Dec. 20, 2023

Related U.S. Application Data
(60) Provisional application No. 63/434,902, filed on Dec. 22, 2022.

(51) Int. Cl. C12Q 1/682; C12Q 1/6876

(52) U.S. Cl. CPC C12Q 1/682; C12Q 1/6876; C12Q 2600/16

(57) ABSTRACT

Disclosed herein, inter alia, are oligonucleotides, methods, and kits useful for amplifying and detecting targets such as nucleic acids, proteins, and carbohydrates.

### CROSS-REFERENCES TO RELATED APPLICATIONS

This application claims the benefit of U.S. Provisional Application No. 63/434,902, filed Dec. 22, 2022, which is incorporated herein by reference in its entirety and for all purposes.

### BACKGROUND

Single-cell technologies have emerged to enable profiling the composition of the genome, epigenome, transcriptome, or proteome of a single cell. Uncovering the distribution, heterogeneity, spatial gene and protein co-expression patterns within cells and tissues is vital for understanding how cell co-localization influences tissue development and the spread of diseases such as cancer, which could lead to important new discoveries and therapeutics. Beyond quantifying gene and protein expression, obtaining precise sequencing information enables identification, monitoring, and possible treatment at the molecular level. Disclosed herein, inter alia, are solutions to these and other problems in the art.

### BRIEF SUMMARY

In an aspect is provided a method of generating an amplification product, the method including: a) hybridizing a first oligonucleotide to a target polynucleotide, wherein the first oligonucleotide includes a first target hybridization sequence, a first probe hybridization sequence, a barcode sequence, and a second probe hybridization sequence; b) hybridizing a second oligonucleotide to the target polynucleotide, wherein the second oligonucleotide includes a second target hybridization sequence, a third probe hybridization sequence, and a fourth probe hybridization sequence; c) hybridizing a first oligonucleotide probe to the first oligonucleotide and to the second oligonucleotide; d) hybridizing a second oligonucleotide probe to the first oligonucleotide and the second oligonucleotide; e) extending the second oligonucleotide probe along the barcode sequence with a polymerase to generate a complementary barcode sequence, ligating the complementary barcode sequence to the first oligonucleotide probe, and ligating the first oligonucleotide probe to the second oligonucleotide probe, thereby generating a circular oligonucleotide; and f) amplifying the circular oligonucleotide, thereby generating an amplification product including multiple complements of the complementary barcode sequence.

In an aspect is provided a method of generating an amplification product, the method including: a) contacting a target polynucleotide with a first oligonucleotide including a first target hybridization sequence, wherein the first oligonucleotide includes a first probe hybridization sequence, a barcode sequence, and a second probe hybridization sequence, and hybridizing the first target hybridization sequence to a first target sequence of the target polynucleotide; b) contacting the target polynucleotide with a second oligonucleotide including a second target hybridization sequence, wherein the second oligonucleotide includes a third probe hybridization sequence and a fourth probe hybridization sequence, and hybridizing the second target hybridization sequence to a second target sequence of the target polynucleotide; c) hybridizing a first oligonucleotide probe to the first oligonucleotide and to the second oligonucleotide, wherein the first oligonucleotide probe includes a sequence complementary to the second probe hybridization sequence and a sequence complementary to the fourth probe hybridization sequence; d) hybridizing a second oligonucleotide probe to the first oligonucleotide and the second oligonucleotide, wherein the second oligonucleotide probe includes a sequence complementary to the first probe hybridization sequence and a sequence complementary to the third probe hybridization sequence; c) extending the second oligonucleotide probe along the barcode sequence with a polymerase to generate a complementary barcode sequence, ligating the complementary barcode sequence to the first oligonucleotide probe, and ligating the first oligonucleotide probe to the second oligonucleotide probe, thereby generating a circular oligonucleotide; and f) amplifying the circular oligonucleotide, thereby generating an amplification product including multiple complements of the complementary barcode sequence.

In an aspect is provided a kit including a first oligonucleotide, a second oligonucleotide, a first oligonucleotide probe, a second oligonucleotide probe, and a ligase, wherein the first oligonucleotide includes a first probe hybridization sequence, a barcode sequence, and a second probe hybridization sequence; the second oligonucleotide includes a third probe hybridization sequence and a fourth probe hybridization sequence; the first oligonucleotide probe includes a sequence complementary to the second probe hybridization sequence and a sequence complementary to the fourth probe hybridization sequence; and the second oligonucleotide probe includes a sequence complementary to the first probe hybridization sequence and a sequence complementary to the third probe hybridization sequence.

### BRIEF DESCRIPTION OF THE DRAWINGS

FIGS. 1A-1E. A cartoon depiction of a cell (1) that is attached to a surface, and is also fixed (e.g., using a fixing agent) and permeabilized according to known methods is illustrated in FIG. 1A. The cell may have been cultured on the surface, or the cell may have been initially cultured in suspension and then fixed to the surface. The RNA molecules (2) present in the cell may be subjected to an amplification technique where a targeted padlock probe (3), which contains an oligonucleotide barcode (4; e.g., 10-15 nucleotides), hybridizes on the RNA. Following padlock probe ligation, the excess content is washed away (e.g., unhybridized padlock probes). As shown in FIG. 1B, the padlock probe is primed across the ligation site (5; i.e., an amplification primer (6) hybridizes to both the 3′ and 5′ ends of the padlock probe) and amplified to produce an amplicon (7). Alternatively, as shown in FIG. 1C, the padlock probe is primed at a region within the padlock probe (i.e., an amplification primer hybridizes a region of the padlock probe) and amplified to produce an amplicon. Here, as well as in FIG. 1B, the ligation site (5) is depicted as a dashed line. The amplicon may be primed with a sequencing primer (8), as depicted in FIG. 1D, and subjected to a sequencing process, whereby the identity of the oligonucleotide barcode, and thus the identity of the RNA molecule is obtained (e.g., via barcode-specific colorimetric signals, 9). FIG. 1E shows the original cell wherein one resolved pixel (depicted by the dotted box) includes a plurality of amplicons. This particular example shows two unique barcodes, each with a unique detection signal (9 and 10, respectively).

FIG. 2. Resolving one pixel of the cell, as depicted in FIGS. 1A-1E, which includes the detection of a plurality of sequencing signals (e.g., fluorescent signals) arising from the barcoded RNA as described according to the methods disclosed herein. Crowding within cells leads to spatial overlap of fluorescence signals within the optically resolved volume when visualized simultaneously. A linear decomposition of the sequencing signal (i.e., the multiplexed signal) into the oligonucleotide barcode basis set allows for the detection and quantitative measurement of multiple RNA in each resolved volume.

FIG. 3. A cartoon depiction of a voxel with the primary coordinate system in Cartesian coordinates. The optically resolved volume has a lateral resolution corresponding to the xy plane, and an axial resolution, corresponding to the z axis as observed in FIG. 3. In embodiments, the dimensions (i.e., the x, y, and z dimensions) of the optically resolved volume are given as (x-dimension)×(y-dimension)×(z-dimension); for example, 0.5 μm×0.5 μm×2 μm.

FIGS. 4A-4B illustrate embodiments of the various oligonucleotides and probes described herein for targeting and detecting polynucleotides. FIG. 4A illustrates an embodiment for detecting a target polynucleotide including a first oligonucleotide (11) and a second oligonucleotide (12), wherein the first oligonucleotide includes a first target hybridization sequence at a 3′ end (13), a first hybridization sequence (14; e.g., a sequence complementary to a second oligonucleotide probe as described in FIG. 4B), a barcode sequence (15), and a second hybridization sequence (16; e.g., a sequence complementary to a first oligonucleotide probe as described in FIG. 4B), and wherein the second oligonucleotide includes a second target hybridization sequence at a 5′ end (17), a third hybridization sequence (18; e.g., a sequence complementary to the second oligonucleotide probe as described in FIG. 4B), and a fourth hybridization sequence (19; e.g., a sequence complementary to the first oligonucleotide probe as described in FIG. 4B). In some embodiment, the 3′ ends of the first and second oligonucleotides are blocked (e.g., reversibly blocked). FIG. 4B illustrates a first oligonucleotide probe (20) and a second oligonucleotide probe (21), wherein the first oligonucleotide probe includes a sequence (22) at a 3′ end complementary to the fourth hybridization sequence of the second oligonucleotide (19) and a sequence at a 5′ end (23) complementary to the second hybridization sequence (16) of the first oligonucleotide as in FIG. 4A, and wherein the second oligonucleotide probe (21) includes a sequence at a 5′ end (24) complementary to the third hybridization sequence (18) of the second oligonucleotide and a sequence at a 3′ end (25) complementary to the first hybridization sequence (14) of the first oligonucleotide as in FIG. 4A. A linker sequence may be present, as illustrated in FIG. 4B, between the first hybridization sequence complement and the third hybridization sequence complement (26), and between the second hybridization sequence complement and the fourth hybridization sequence complement (27).

FIGS. 5A-5D illustrate an embodiment of the invention herein for detecting a plurality of target polynucleotides. FIG. 5A illustrates a first set of three independently targeted pairs of oligonucleotide probes (e.g., first and second oligonucleotides as described in FIG. 4A) hybridized to a first mRNA molecule (28) and a second set of three independently targeted pairs of oligonucleotide probes hybridized to a second mRNA molecule (29), wherein for each set, the first oligonucleotide of each pair includes a unique barcode (e.g., 30 or 31). Once each pair of first and second oligonucleotides are hybridized to the target regions (e.g., via target sequences, labeled as 32-37 in FIG. 5A), a first oligonucleotide probe and a second oligonucleotide probe (as described in FIG. 4B) are hybridized to each pair of first and second oligonucleotides, as shown in FIG. 5B, wherein the 5′ end of the hybridized first oligonucleotide probe (23) and the 3′ end of the hybridized second oligonucleotide probe (25) flanks the barcode sequence of the first oligonucleotide (30 or 31). A gap (e.g., a nick) is also present between the 3′ end of the first oligonucleotide probe (22) and the 5′ end of the second oligonucleotide probe (24). Gap-filling is then performed with a polymerase to incorporate a complementary barcode sequence (38 or 39) into the 3′ end of the second oligonucleotide probe, as shown in FIG. 5C, followed by ligation of the nick and the complementary barcode sequence to the first oligonucleotide probe to generate a circular oligonucleotide. Following gap filling and ligation, this circular oligonucleotide may be amplified, for example, by rolling circle amplification (RCA) using a strand-displacing polymerase (40) and the 3′ end of the second oligonucleotide as an amplification primer, as shown in FIG. 5D.

### DETAILED DESCRIPTION

The aspects and embodiments described herein relate to polynucleotide probes, methods, and kits useful for amplifying and detecting target nucleic acids, optionally within a cell or in situ.

### I. Definitions

All patents, patent applications, articles and publications mentioned herein, both supra and infra, are hereby expressly incorporated herein by reference in their entireties. The practice of the technology described herein will employ, unless indicated specifically to the contrary, conventional methods of chemistry, biochemistry, organic chemistry, molecular biology, bioinformatics, microbiology, recombinant DNA techniques, genetics, immunology, and cell biology that are within the skill of the art, many of which are described below for the purpose of illustration. Examples of such techniques are available in the literature. Sec, e.g., Singleton et al., DICTIONARY OF MICROBIOLOGY AND MOLECULAR BIOLOGY 2nd ed., J. Wiley & Sons (New York, NY 1994); and Sambrook and Green, Molecular Cloning: A Laboratory Manual, 4th Edition (2012). Methods, devices and materials similar or equivalent to those described herein can be used in the practice of this invention.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art. The following definitions are provided to facilitate understanding of certain terms used frequently herein and are not meant to limit the scope of the present disclosure.

As used herein, the singular terms “a”, “an”, and “the” include the plural reference unless the context clearly indicates otherwise. Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein, the term “control” or “control experiment” is used in accordance with its plain and ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

As used herein, the term “complement” is used in accordance with its plain and ordinary meaning and refers to a nucleotide (e.g., RNA nucleotide or DNA nucleotide) or a sequence of nucleotides capable of base pairing with a complementary nucleotide or sequence of nucleotides (e.g., Watson-Crick base pairing). As described herein and commonly known in the art the complementary (matching) nucleotide of adenosine is thymidine and the complementary (matching) nucleotide of guanosine is cytosine. Thus, a complement may include a sequence of nucleotides that base paired with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence. Another example of complementary sequences are a template sequence and an amplicon sequence polymerized by a polymerase along the template sequence. “Duplex” means at least two oligonucleotides and/or polynucleotides that are fully or partially complementary undergo Watson-Crick type base pairing among all or most of their nucleotides so that a stable complex is formed. Complementary single stranded nucleic acids and/or substantially complementary single stranded nucleic acids can hybridize to each other under hybridization conditions, thereby forming a nucleic acid that is partially or fully double stranded. When referring to a double-stranded polynucleotide including a first strand hybridized to a second strand, it is understood that each of the first strand and the second strand are independently single-stranded polynucleotides. All or a portion of a nucleic acid sequence may be substantially complementary to another nucleic acid sequence, in some embodiments. As referred to herein, “substantially complementary” refers to nucleotide sequences that can hybridize with each other under suitable hybridization conditions. Hybridization conditions can be altered to tolerate varying amounts of sequence mismatch within complementary nucleic acids that are substantially complementary. Substantially complementary portions of nucleic acids that can hybridize to each other can be 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other. In some embodiments substantially complementary portions of nucleic acids that can hybridize to each other are 100% complementary. Nucleic acids, or portions thereof, that are configured to hybridize to each other often include nucleic acid sequences that are substantially complementary to each other.

As described herein, the complementarity of sequences may be partial, in which only some of the nucleic acids match according to base pairing, or complete, where all the nucleic acids match according to base pairing. Thus, two sequences that are complementary to each other, may have a specified percentage of nucleotides that complement one another (e.g., about 60%, preferably 65%, 70%, 75%, 80%, 85%, 90%, 91%, 92%, 93%, 94%, 95%, 96%, 97%, 98%, 99%, or higher complementarity over a specified region). In embodiments, two sequences are complementary when they are completely complementary, having 100% complementarity. In embodiments, sequences in a pair of complementary sequences form portions of a single polynucleotide with non-base-pairing nucleotides (e.g., as in a hairpin or loop structure, with or without an overhang) or portions of separate polynucleotides. In embodiments, one or both sequences in a pair of complementary sequences form portions of longer polynucleotides, which may or may not include additional regions of complementarity.

As used herein, the term “contacting” is used in accordance with its plain ordinary meaning and refers to the process of allowing at least two distinct species (e.g., chemical compounds including biomolecules, particles, solid supports, or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be a compound as described herein and a protein or enzyme.

As may be used herein, the terms “nucleic acid,” “nucleic acid molecule,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may include natural nucleic acid sequences and variants thereof, artificial nucleic acid sequences, or a combination of such sequences. As may be used herein, the terms “nucleic acid oligomer” and “oligonucleotide” are used interchangeably and are intended to include, but are not limited to, nucleic acids having a length of 200 nucleotides or less. In some embodiments, an oligonucleotide is a nucleic acid having a length of 2 to 200 nucleotides, 2 to 150 nucleotides, 5 to 150 nucleotides or 5 to 100 nucleotides. The terms “polynucleotide,” “oligonucleotide,” “oligo” or the like refer, in the usual and customary sense, to a linear sequence of nucleotides. Oligonucleotides are typically from about 5, 6, 7, 8, 9, 10, 12, 15, 25, 30, 40, 50 or more nucleotides in length, up to about 100 nucleotides in length. In some embodiments, an oligonucleotide is a primer configured for extension by a polymerase when the primer is annealed completely or partially to a complementary nucleic acid template. A primer is often a single stranded nucleic acid. In certain embodiments, a primer, or portion thereof, is substantially complementary to a portion of an adapter. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. In some embodiments, an oligonucleotide may be immobilized to a solid support.

As used herein, the terms “polynucleotide primer” and “primer” refers to any polynucleotide molecule that may hybridize to a polynucleotide template, be bound by a polymerase, and be extended in a template-directed process for nucleic acid synthesis (e.g., amplification and/or sequencing). The primer may be a separate polynucleotide from the polynucleotide template, or both may be portions of the same polynucleotide (e.g., as in a hairpin structure having a 3′ end that is extended along another portion of the polynucleotide to extend a double-stranded portion of the hairpin). Primers (e.g., forward or reverse primers) may be attached to a solid support. A primer can be of any length depending on the particular technique it will be used for. For example, PCR primers are generally between 10 and 40 nucleotides in length. The length and complexity of the nucleic acid fixed onto the nucleic acid template may vary. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. In certain embodiments, a primer has a length of 10 to 150 nucleotides, 15 to 150 nucleotides, 5 to 100 nucleotides, 5 to 50 nucleotides or 10 to 50 nucleotides. A primer typically has a length of 10 to 50 nucleotides. For example, a primer may have a length of 10 to 40, 10 to 30, 10 to 20, 25 to 50, 15 to 40, 15 to 30, 20 to 50, 20 to 40, or 20 to 30 nucleotides. In some embodiments, a primer has a length of 18 to 24 nucleotides. One of skill can adjust these factors to provide optimum hybridization and signal production for a given hybridization procedure. The primer permits the addition of a nucleotide residue thereto, or oligonucleotide or polynucleotide synthesis therefrom, under suitable conditions. In an embodiment the primer is a DNA primer, i.e., a primer consisting of, or largely consisting of, deoxyribonucleotide residues. The primers are designed to have a sequence that is the complement of a region of template/target DNA to which the primer hybridizes. The addition of a nucleotide residue to the 3′ end of a primer by formation of a phosphodiester bond results in a DNA extension product. The addition of a nucleotide residue to the 3′ end of the DNA extension product by formation of a phosphodiester bond results in a further DNA extension product. In another embodiment the primer is an RNA primer. In embodiments, a primer is hybridized to a target polynucleotide. A “primer” is complementary to a polynucleotide template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

As used herein, the term “primer binding sequence” refers to a polynucleotide sequence that is complementary to at least a portion of a primer (e.g., a sequencing primer or an amplification primer). Primer binding sequences can be of any suitable length. In embodiments, a primer binding sequence is about or at least about 10, 15, 20, 25, 30, or more nucleotides in length. In embodiments, a primer binding sequence is 10-50, 15-30, or 20-25 nucleotides in length. The primer binding sequence may be selected such that the primer (e.g., sequencing primer) has the preferred characteristics to minimize secondary structure formation or minimize non-specific amplification, for example having a length of about 20-30 nucleotides; approximately 50% GC content, and a Tm of about 55° C. to about 65° C.

Nucleic acids, including e.g., nucleic acids with a phosphorothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

As used herein, a platform primer is a primer oligonucleotide immobilized or otherwise bound to a solid support (i.e. an immobilized oligonucleotide). Examples of platform primers include P7 and P5 primers (i.e., Illumina® platform sequences), or S1 and S2 primers (i.e., Singular Genomics® platform sequences), or the reverse complements thereof. A “platform primer binding sequence” refers to a sequence or portion of an oligonucleotide that is capable of binding to a platform primer (e.g., the platform primer binding sequence is complementary to the platform primer). In embodiments, a platform primer binding sequence may form part of an adapter. In embodiments, a platform primer binding sequence is complementary to a platform primer sequence. In embodiments, a platform primer binding sequence is complementary to a primer.

The order of elements within a nucleic acid molecule is typically described herein from 5′ to 3′. In the case of a double-stranded molecule, the “top” strand is typically shown from 5′ to 3′, according to convention, and the order of elements is described herein with reference to the top strand.

The term “messenger RNA” or “mRNA” refers to an RNA that is without introns and is capable of being translated into a polypeptide. The term “RNA” refers to any ribonucleic acid, including but not limited to mRNA, tRNA (transfer RNA), rRNA (ribosomal RNA), and/or noncoding RNA (such as lncRNA (long noncoding RNA)). The term “cDNA” refers to a DNA that is complementary or identical to an RNA, in either single stranded or double stranded form.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

As used herein, the term “associated” or “associated with” can mean that two or more species are identifiable as being co-located at a point in time. An association can mean that two or more species are or were within a similar container. An association can be an informatics association, where for example digital information regarding two or more species is stored and can be used to determine that one or more of the species were co-located at a point in time. An association can also be a physical association. In some instances two or more associated species are “tethered”, “coated”, “attached”, or “immobilized” to one another or to a common solid or semisolid support (e.g. a receiving substrate). An association may refer to a relationship, or connection, between two entities. For example, a barcode sequence may be associated with a particular target by binding a probe including the barcode sequence to the target. In embodiments, detecting the associated barcode provides detection of the target. Associated may refer to the relationship between a sample and the DNA molecules, RNA molecules, or polynucleotides originating from or derived from that sample. These relationships may be encoded in oligonucleotide barcodes, as described herein. A polynucleotide is associated with a sample if it is an endogenous polynucleotide, i.e., it occurs in the sample at the time the sample is obtained, or is derived from an endogenous polynucleotide. For example, the RNAs endogenous to a cell are associated with that cell. cDNAs resulting from reverse transcription of these RNAs, and DNA amplicons resulting from PCR amplification of the cDNAs, contain the sequences of the RNAs and are also associated with the cell. The polynucleotides associated with a sample need not be located or synthesized in the sample, and are considered associated with the sample even after the sample has been destroyed (for example, after a cell has been lysed). Barcoding can be used to determine which polynucleotides in a mixture are associated with a particular sample. In embodiments, a proximity probe is associated with a particular barcode, such that identifying the barcode identifies the probe with which it is associated. Because the proximity probe specifically binds to a target, identifying the barcode thus identifies the target.

The term “adapter” as used herein refers to any oligonucleotide that can be ligated to a nucleic acid molecule, thereby generating nucleic acid products that can be sequenced on a sequencing platform (e.g., an Illumina® or Singular Genomics G4® sequencing platform). In embodiments, adapters include two reverse complementary oligonucleotides forming a double-stranded structure. In embodiments, an adapter includes two oligonucleotides that are complementary at one portion and mismatched at another portion, forming a Y-shaped or fork-shaped adapter that is double stranded at the complementary portion and has two overhangs at the mismatched portion. Since Y-shaped adapters have a complementary, double-stranded region, they can be considered a special form of double-stranded adapters. When this disclosure contrasts Y-shaped adapters and double stranded adapters, the term “double-stranded adapter” or “blunt-ended” is used to refer to an adapter having two strands that are fully complementary, substantially (e.g., more than 90% or 95%) complementary, or partially complementary. In embodiments, adapters include sequences that bind to sequencing primers. In embodiments, adapters include sequences that bind to immobilized oligonucleotides (e.g., P7 and P5 sequences) or reverse complements thereof. In embodiments, the adapter is substantially non-complementary to the 3′ end or the 5′ end of any target polynucleotide present in the sample. In embodiments, the adapter can include a sequence that is substantially identical, or substantially complementary, to at least a portion of a primer, for example a universal primer. In embodiments, the adapter can include an index sequence (also referred to as barcode or tag) to assist with downstream error correction, identification or sequencing. In some embodiments, an adapter is hairpin adapter (also referred to herein as a hairpin). In some embodiments, a hairpin adapter includes a single nucleic acid strand including a stem-loop structure. In some embodiments, a hairpin adapter includes a nucleic acid having a 5′-end, a 5′-portion, a loop, a 3′-portion and a 3′-end (e.g., arranged in a 5′ to 3′ orientation). In some embodiments, the 5′ portion of a hairpin adapter is annealed and/or hybridized to the 3′ portion of the hairpin adapter, thereby forming a stem portion of the hairpin adapter. In some embodiments, the 5′ portion of a hairpin adapter is substantially complementary to the 3′ portion of the hairpin adapter. In certain embodiments, a hairpin adapter includes a stem portion (i.e., stem) and a loop, wherein the stem portion is substantially double stranded thereby forming a duplex. In some embodiments, the loop of a hairpin adapter includes a nucleic acid strand that is not complementary (e.g., not substantially complementary) to itself or to any other portion of the hairpin adapter. In some embodiments, a method herein includes ligating a first adapter to a first end of a double stranded nucleic acid, and ligating a second adapter to a second end of a double stranded nucleic acid. In some embodiments, the first adapter and the second adapter are different. For example, in certain embodiments, the first adapter and the second adapter may include different nucleic acid sequences or different structures. In some embodiments, the first adapter is a Y-adapter and the second adapter is a hairpin adapter. In some embodiments, the first adapter is a hairpin adapter and a second adapter is a hairpin adapter. In certain embodiments, the first adapter and the second adapter may include different primer binding sites, different structures, and/or different capture sequences (e.g., a sequence complementary to a capture nucleic acid). In some embodiments, some, all or substantially all of the nucleic acid sequence of a first adapter and a second adapter are the same. In some embodiments, some, all or substantially all of the nucleic acid sequence of a first adapter and a second adapter are substantially different.

As used herein, the terms “analogue” and “analog”, in reference to a chemical compound, refers to compound having a structure similar to that of another one, but differing from it in respect of one or more different atoms, functional groups, or substructures that are replaced with one or more other atoms, functional groups, or substructures. In the context of a nucleotide, a nucleotide analog refers to a compound that, like the nucleotide of which it is an analog, can be incorporated into a nucleic acid molecule (e.g., an extension product) by a suitable polymerase, for example, a DNA polymerase in the context of a nucleotide analogue. The terms also encompass nucleic acids containing known nucleotide analogs or modified backbone residues or linkages, which are synthetic, naturally occurring, or non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphorothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see, e.g., see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA)), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

As used herein, a “native” nucleotide is used in accordance with its plain and ordinary meaning and refers to a naturally occurring nucleotide that does not include an exogenous label (e.g., a fluorescent dye, or other label) or chemical modification such as may characterize a nucleotide analog. Examples of native nucleotides useful for carrying out procedures described herein include: dATP (2′-deoxyadenosine-5′-triphosphate); dGTP (2′-deoxyguanosine-5′-triphosphate); dCTP (2′-deoxycytidine-5′-triphosphate); dTTP (2′-deoxythymidine-5′-triphosphate); and dUTP (2′-deoxyuridine-5′-triphosphate).

In embodiments, the nucleotides of the present disclosure use a cleavable linker to attach the label to the nucleotide. The use of a cleavable linker ensures that the label can, if required, be removed after detection, avoiding any interfering signal with any labelled nucleotide incorporated subsequently. The use of the term “cleavable linker” is not meant to imply that the whole linker is required to be removed from the nucleotide base. The cleavage site can be located at a position on the linker that ensures that part of the linker remains attached to the nucleotide base after cleavage. The linker can be attached at any position on the nucleotide base provided that Watson-Crick base pairing can still be carried out. In the context of purine bases, it is preferred if the linker is attached via the 7-position of the purine or the preferred deazapurine analogue, via an 8-modified purine, via an N-6 modified adenosine or an N-2 modified guanine. For pyrimidines, attachment is preferably via the 5-position on cytidine, thymidine or uracil and the N-4 position on cytosine.

The term “cleavable linker” or “cleavable moiety” as used herein refers to a divalent or monovalent, respectively, moiety which is capable of being separated (e.g., detached, split, disconnected, hydrolyzed, a stable bond within the moiety is broken) into distinct entities. A cleavable linker is cleavable (e.g., specifically cleavable) in response to external stimuli (e.g., enzymes, nucleophilic/basic reagents, reducing agents, photo-irradiation, electrophilic/acidic reagents, organometallic and metal reagents, or oxidizing reagents). A chemically cleavable linker refers to a linker which is capable of being split in response to the presence of a chemical (e.g., acid, base, oxidizing agent, reducing agent, Pd(0), tris-(2-carboxyethyl)phosphine, dilute nitrous acid, fluoride, tris(3-hydroxypropyl)phosphine), sodium dithionite (Na₂S₂O₄), or hydrazine (N₂H₄)). A chemically cleavable linker is non-enzymatically cleavable. In embodiments, the cleavable linker is cleaved by contacting the cleavable linker with a cleaving agent. In embodiments, the cleaving agent is a phosphine containing reagent (e.g., TCEP or THPP), sodium dithionite (Na₂S₂O₄), weak acid, hydrazine (N₂H₄), Pd(0), or light-irradiation (e.g., ultraviolet radiation). In embodiments, cleaving includes removing. A “cleavable site” or “scissile linkage” in the context of a polynucleotide is a site which allows controlled cleavage of the polynucleotide strand (e.g., the linker, the primer, or the polynucleotide) by chemical, enzymatic, or photochemical means known in the art and described herein. A scissile site may refer to the linkage of a nucleotide between two other nucleotides in a nucleotide strand (i.e., an internucleosidic linkage). In embodiments, the scissile linkage can be located at any position within the one or more nucleic acid molecules, including at or near a terminal end (e.g., the 3′ end of an oligonucleotide) or in an interior portion of the one or more nucleic acid molecules. In embodiments, conditions suitable for separating a scissile linkage include a modulating the pH and/or the temperature. In embodiments, a scissile site can include at least one acid-labile linkage. For example, an acid-labile linkage may include a phosphoramidate linkage. In embodiments, a phosphoramidate linkage can be hydrolysable under acidic conditions, including mild acidic conditions such as trifluoroacetic acid and a suitable temperature (e.g., 30° C.), or other conditions known in the art, for example Matthias Mag, et al Tetrahedron Letters, Volume 33, Issue 48, 1992, 7319-7322. In embodiments, the scissile site can include at least one photolabile internucleosidic linkage (e.g., o-nitrobenzyl linkages, as described in Walker et al, J. Am. Chem. Soc. 1988, 110, 21, 7170-7177), such as o-nitrobenzyloxymethyl or p-nitrobenzyloxymethyl group(s). In embodiments, the scissile site includes at least one uracil nucleobase. In embodiments, a uracil nucleobase can be cleaved with a uracil DNA glycosylase (UDG) or Formamidopyrimidine DNA Glycosylase Fpg. In embodiments, the scissile linkage site includes a sequence-specific nicking site having a nucleotide sequence that is recognized and nicked by a nicking endonuclease enzyme or a uracil DNA glycosylase.

As used herein, the term “modified nucleotide” refers to nucleotide modified in some manner. Typically, a nucleotide contains a single 5-carbon sugar moiety, a single nitrogenous base moiety and 1 to three phosphate moieties. In embodiments, a nucleotide can include a blocking moiety and/or a label moiety. A blocking moiety on a nucleotide prevents formation of a covalent bond between the 3′ hydroxyl moiety of the nucleotide and the 5′ phosphate of another nucleotide. A blocking moiety on a nucleotide can be reversible, whereby the blocking moiety can be removed or modified to allow the 3′ hydroxyl to form a covalent bond with the 5′ phosphate of another nucleotide. A blocking moiety can be effectively irreversible under particular conditions used in a method set forth herein. In embodiments, the blocking moiety is attached to the 3′ oxygen of the nucleotide and is independently —NH₂, —CN, —CH₃, C₂-C₆ allyl (e.g., —CH₂—CH═CH₂), methoxyalkyl (e.g., —CH₂—O—CH₃), or —CH₂N₃. In embodiments, the blocking moiety is attached to the 3′ oxygen of the nucleotide and is independently

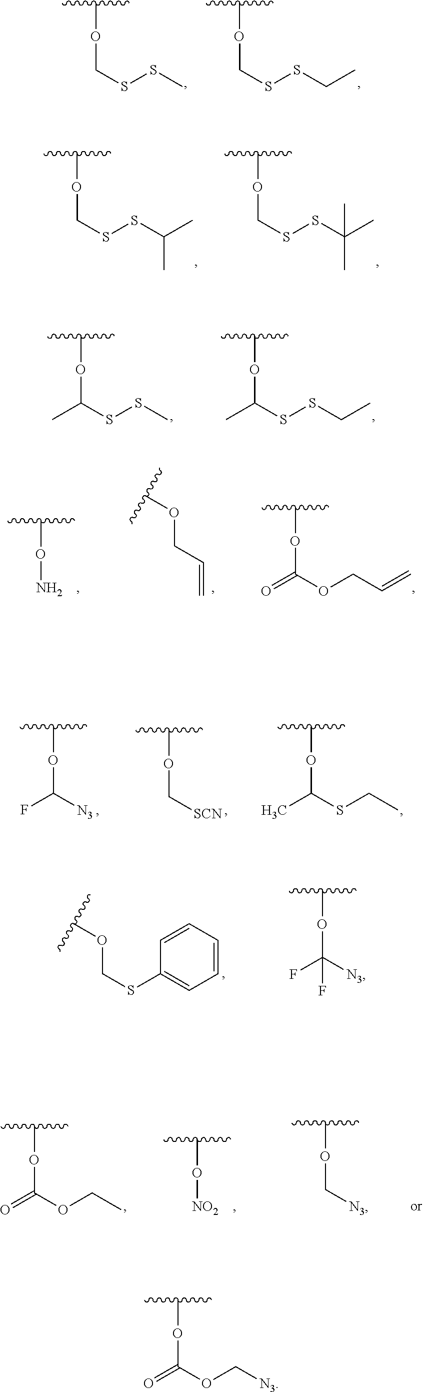

A label moiety of a modified nucleotide can be any moiety that allows the nucleotide to be detected, for example, using a spectroscopic method. Exemplary label moieties are fluorescent labels, mass labels, chemiluminescent labels, electrochemical labels, detectable labels and the like. One or more of the above moieties can be absent from a nucleotide used in the methods and compositions set forth herein. For example, a nucleotide can lack a label moiety or a blocking moiety or both. Examples of nucleotide analogues include, without limitation, 7-deaza-adenine, 7-deaza-guanine, the analogues of deoxynucleotides shown herein, analogues in which a label is attached through a cleavable linker to the 5-position of cytosine or thymine or to the 7-position of deaza-adenine or deaza-guanine, and analogues in which a small chemical moiety is used to cap the OH group at the 3′-position of deoxyribose. Nucleotide analogues and DNA polymerase-based DNA sequencing are also described in U.S. Pat. No. 6,664,079, which is incorporated herein by reference in its entirety for all purposes. Non-limiting examples of detectable labels include labels including fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In embodiments, the dye is a fluorescent dye. Non-limiting examples of dyes, some of which are commercially available, include CF dyes (Biotium, Inc.), Alexa Fluor dyes (Thermo Fisher), DyLight dyes (Thermo Fisher), Cy dyes (GE Healthscience), IRDyes (Li-Cor Biosciences, Inc.), and HiLyte dyes (Anaspec, Inc.). In embodiments, the label is a fluorophore.

In some embodiments, a nucleic acid includes a label. As used herein, the term “label” or “labels” is used in accordance with their plain and ordinary meanings and refer to molecules that can directly or indirectly produce or result in a detectable signal either by themselves or upon interaction with another molecule. Non-limiting examples of detectable labels include fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In embodiments, the label is a dye. In embodiments, the dye is a fluorescent dye. Non-limiting examples of dyes, some of which are commercially available, include CF dyes (Biotium, Inc.), Alexa Fluor dyes (Thermo Fisher), DyLight dyes (Thermo Fisher), Cy dyes (GE Healthscience), IRDyes (Li-Cor Biosciences, Inc.), and HiLyte dyes (Anaspec, Inc.). In embodiments, a particular nucleotide type is associated with a particular label, such that identifying the label identifies the nucleotide with which it is associated. In embodiments, the label is luciferin that reacts with luciferase to produce a detectable signal in response to one or more bases being incorporated into an elongated complementary strand, such as in pyrosequencing. In embodiment, a nucleotide includes a label (such as a dye). In embodiments, the label is not associated with any particular nucleotide, but detection of the label identifies whether one or more nucleotides having a known identity were added during an extension step (such as in the case of pyrosequencing). Examples of detectable agents (i.e., labels) include imaging agents, including fluorescent and luminescent substances, molecules, or compositions, including, but not limited to, a variety of organic or inorganic small molecules commonly referred to as “dyes,” “labels,” or “indicators.” Examples include fluorescein, rhodamine, acridine dyes, Alexa dyes, and cyanine dyes. In embodiments, the detectable moiety is a fluorescent molecule (e.g., acridine dye, cyanine, dye, fluorine dye, oxazine dye, phenanthridine dye, or rhodamine dye). In embodiments, the detectable moiety is a fluorescent molecule (e.g., acridine dye, cyanine, dye, fluorine dye, oxazine dye, phenanthridine dye, or rhodamine dye). The term “cyanine” or “cyanine moiety” as described herein refers to a detectable moiety containing two nitrogen groups separated by a polymethine chain. In embodiments, the cyanine moiety has 3 methine structures (i.e., cyanine 3 or Cy3). In embodiments, the cyanine moiety has 5 methine structures (i.e., cyanine 5 or Cy5). In embodiments, the cyanine moiety has 7 methine structures (i.e., cyanine 7 or Cy7).

The term “nucleoside” refers, in the usual and customary sense, to a glycosylamine including a nucleobase and a five-carbon sugar (ribose or deoxyribose). Non-limiting examples of nucleosides include cytidine, uridine, adenosine, guanosine, thymidine and inosine. Nucleosides may be modified at the base and/or the sugar. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g., polynucleotides contemplated herein include any types of RNA, e.g., mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (sec, e.g., NCBI web site www.ncbi.nlm.nih.gov/BLAST/or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

As used herein, the term “removable” group, e.g., a label or a blocking group or protecting group, is used in accordance with its plain and ordinary meaning and refers to a chemical group that can be removed from a nucleotide analogue such that a DNA polymerase can extend the nucleic acid (e.g., a primer or extension product) by the incorporation of at least one additional nucleotide. Removal may be by any suitable method, including enzymatic, chemical, or photolytic cleavage. Removal of a removable group, e.g., a blocking group, does not require that the entire removable group be removed, only that a sufficient portion of it be removed such that a DNA polymerase can extend a nucleic acid by incorporation of at least one additional nucleotide using a nucleotide or nucleotide analogue. In general, the conditions under which a removable group is removed are compatible with a process employing the removable group (e.g., an amplification process or sequencing process).

As used herein, the terms “reversible blocking groups” and “reversible terminators” are used in accordance with their plain and ordinary meanings and refer to a blocking moiety located, for example, at the 3′ position of a modified nucleotide and may be a chemically cleavable moiety such as an allyl group, an azidomethyl group or a methoxymethyl group, or may be an enzymatically cleavable group such as a phosphate ester. Non-limiting examples of nucleotide blocking moieties are described in applications WO 2004/018497, WO 96/07669, U.S. Pat. Nos. 7,057,026, 7,541,444, 5,763,594, 5,808,045, 5,872,244 and 6,232,465 the contents of which are incorporated herein by reference in their entirety. The nucleotides may be labelled or unlabeled. They may be modified with reversible terminators useful in methods provided herein and may be 3′-O-blocked reversible or 3′-unblocked reversible terminators. In nucleotides with 3′-O-blocked reversible terminators, the blocking group —OR [reversible terminating (capping) group] is linked to the oxygen atom of the 3′-OH of the pentose, while the label is linked to the base, which acts as a reporter and can be cleaved. The 3′-O-blocked reversible terminators are known in the art, and may be, for instance, a 3′-ONH₂ reversible terminator, a 3′-O-allyl reversible terminator, or a 3′-O-azidomethyl reversible terminator. In embodiments, the reversible terminator moiety is attached to the 3′-oxygen of the nucleotide, having the formula:

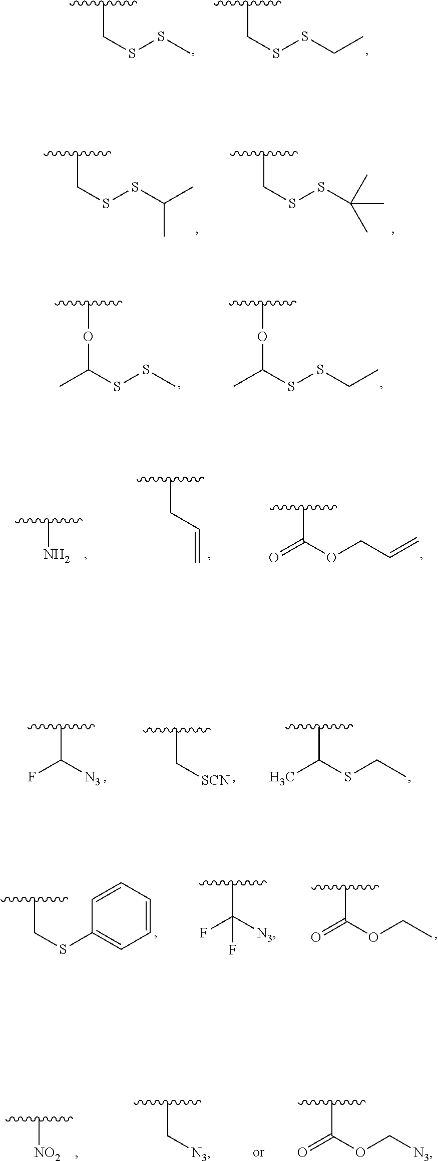

wherein the 3′ oxygen of the nucleotide is not shown in the formulae above. The term “allyl” as described herein refers to an unsubstituted methylene attached to a vinyl group (i.e., —CH—CH₂). In embodiments, the reversible terminator moiety is

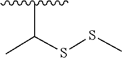 [SMILES: *C(C)SSC]

as described in U.S. Pat. No. 10,738,072, which is incorporated herein by reference for all purposes. For example, a nucleotide including a reversible terminator moiety may be represented by the formula:

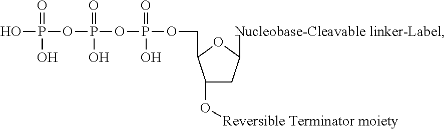

where the nucleobase is adenine or adenine analogue, thymine or thymine analogue, guanine or guanine analogue, or cytosine or cytosine analogue.

In some embodiments, a nucleic acid (e.g., a probe or a primer) includes a molecular identifier or a molecular barcode. As used herein, the term “molecular barcode” (which may be referred to as a “tag”, a “barcode”, a “molecular identifier”, an “identifier sequence” or a “unique molecular identifier” (UMI)) refers to any material (e.g., a nucleotide sequence, a nucleic acid molecule feature) that is capable of distinguishing an individual molecule in a large heterogeneous population of molecules. In embodiments, a barcode is unique in a pool of barcodes that differ from one another in sequence, or is uniquely associated with a particular sample polynucleotide in a pool of sample polynucleotides. In embodiments, every barcode in a pool of adapters is unique, such that sequencing reads including the barcode can be identified as originating from a single sample polynucleotide molecule on the basis of the barcode alone. In other embodiments, individual barcode sequences may be used more than once, but adapters including the duplicate barcodes are associated with different sequences and/or in different combinations of barcoded adaptors, such that sequence reads may still be uniquely distinguished as originating from a single sample polynucleotide molecule on the basis of a barcode and adjacent sequence information (e.g., sample polynucleotide sequence, and/or one or more adjacent barcodes). In embodiments, barcodes are about or at least about 5, 6, 7, 8, 9, 10, 15, 20, 25, 30, 40, 50, 75 or more nucleotides in length. In embodiments, barcodes are shorter than 20, 15, 10, 9, 8, 7, 6, or 5 nucleotides in length. In embodiments, barcodes are about 10 to about 50 nucleotides in length, such as about 15 to about 40 or about 20 to about 30 nucleotides in length. In a pool of different barcodes, barcodes may have the same or different lengths. In general, barcodes are of sufficient length and include sequences that are sufficiently different to allow the identification of sequencing reads that originate from the same sample polynucleotide molecule. In embodiments, each barcode in a plurality of barcodes differs from every other barcode in the plurality by at least three nucleotide positions, such as at least 3, 4, 5, 6, 7, 8, 9, 10, or more nucleotide positions. In some embodiments, substantially degenerate barcodes may be known as random. In some embodiments, a barcode may include a nucleic acid sequence from within a pool of known sequences. In some embodiments, the barcodes may be pre-defined. In embodiments, the barcodes are selected to form a known set of barcodes, e.g., the set of barcodes may be distinguished by a particular Hamming distance. In embodiments, each barcode sequence is unique within the known set of barcodes. In embodiments, each barcode sequence is associated with a particular oligonucleotide probe.

In embodiments, a nucleic acid (e.g., an adapter or primer) includes a sample barcode. In general, a “sample barcode” is a nucleotide sequence that is sufficiently different from other sample barcode to allow the identification of the sample source based on sample barcode sequence(s) with which they are associated. In embodiments, a plurality of nucleotides (e.g., all nucleotides from a particular sample source, or sub-sample thereof) are joined to a first sample barcode, while a different plurality of nucleotides (e.g., all nucleotides from a different sample source, or different subsample) are joined to a second sample barcode, thereby associating each plurality of polynucleotides with a different sample barcode indicative of sample source. In embodiments, each sample barcode in a plurality of sample barcodes differs from every other sample barcode in the plurality by at least three nucleotide positions, such as at least 3, 4, 5, 6, 7, 8, 9, 10, or more nucleotide positions. In some embodiments, substantially degenerate sample barcodes may be known as random. In some embodiments, a sample barcode may include a nucleic acid sequence from within a pool of known sequences. In some embodiments, the sample barcodes may be pre-defined. In embodiments, the sample barcode includes about 1 to about 10 nucleotides. In embodiments, the sample barcode includes about 3, 4, 5, 6, 7, 8, 9, or about 10 nucleotides. In embodiments, the sample barcode includes about 3 nucleotides. In embodiments, the sample barcode includes about 5 nucleotides. In embodiments, the sample barcode includes about 7 nucleotides. In embodiments, the sample barcode includes about 10 nucleotides. In embodiments, the sample barcode includes about 6 to about 10 nucleotides.

As used herein, the term “DNA polymerase” and “nucleic acid polymerase” are used in accordance with their plain ordinary meanings and refer to enzymes capable of synthesizing nucleic acid molecules from nucleotides (e.g., deoxyribonucleotides). Exemplary types of polymerases that may be used in the compositions and methods of the present disclosure include the nucleic acid polymerases such as DNA polymerase, DNA- or RNA-dependent RNA polymerase, and reverse transcriptase. In some cases, the DNA polymerase is 9° N polymerase or a variant thereof, E. Coli DNA polymerase I, Bacteriophage T4 DNA polymerase, Sequenase, Taq DNA polymerase, DNA polymerase from Bacillus stearothermophilus, Bst 2.0 DNA polymerase, 9° N polymerase (exo-) A485L/Y409V, Phi29 DNA Polymerase (429 DNA Polymerase), T7 DNA polymerase, DNA polymerase II, DNA polymerase III holoenzyme, DNA polymerase IV, DNA polymerase V, VentR DNA polymerase, Therminator™ II DNA Polymerase, Therminator™ III DNA Polymerase, or Therminator™ IX DNA Polymerase. In embodiments, the polymerase is a protein polymerase. Typically, a DNA polymerase adds nucleotides to the 3′-end of a DNA strand, one nucleotide at a time. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol μ DNA polymerase, Pol λ DNA polymerase, Pol σ DNA polymerase, Pol α DNA polymerase, Pol δ DNA polymerase, Pol ε DNA polymerase, Pol η DNA polymerase, Pol ι DNA polymerase, Pol κ DNA polymerase, Pol ζ DNA polymerase, Pol γ DNA polymerase, Pol θ DNA polymerase, Pol ν DNA polymerase, or a thermophilic nucleic acid polymerase (e.g. Therminator γ, 9° N polymerase (exo-), Therminator II, Therminator III, or Therminator IX). In embodiments, the DNA polymerase is a modified archacal DNA polymerase. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044). In embodiments, the polymerase is an enzyme described in US 2021/0139884. For example, a polymerase catalyzes the addition of a next correct nucleotide to the 3′-OH group of the primer via a phosphodiester bond, thereby chemically incorporating the nucleotide into the primer. Optionally, the polymerase used in the provided methods is a processive polymerase. Optionally, the polymerase used in the provided methods is a distributive polymerase.

As used herein, the term “thermophilic nucleic acid polymerase” refers to a family of DNA polymerases (e.g., 9° N™) and mutants thereof derived from the DNA polymerase originally isolated from the hyperthermophilic archaca, Thermococcus sp. 9 degrees N-7, found in hydrothermal vents at that latitude (East Pacific Rise) (Southworth M W, et al. PNAS. 1996; 93(11):5281-5285). A thermophilic nucleic acid polymerase is a member of the family B DNA polymerases. Site-directed mutagenesis of the 3′-5′ exo motif I (Asp-Ile-Glu or DIE) to AIA, AIE, EIE, EID or DIA yielded polymerase with no detectable 3′ exonuclease activity. Mutation to Asp-Ile-Asp (DID) resulted in reduction of 3′-5′ exonuclease specific activity to <1% of wild type, while maintaining other properties of the polymerase including its high strand displacement activity. The sequence AIA (D141A, E143A) was chosen for reducing exonuclease. Subsequent mutagenesis of key amino acids results in an increased ability of the enzyme to incorporate dideoxynucleotides, ribonucleotides and acyclonucleotides (e.g., Therminator II enzyme from New England Biolabs with D141A/E143A/Y409V/A485L mutations); 3′-amino-dNTPs, 3′-azido-dNTPs and other 3′-modified nucleotides (e.g., NEB Therminator III DNA Polymerase with D141A/E143A/L408S/Y409A/P410V mutations, NEB Therminator IX DNA polymerase), or γ-phosphate labeled nucleotides (e.g., Therminator γ: D141A/E143A/W355A/L408 W/R460A/Q461S/K464E/D480V/R484 W/A485L). Typically, these enzymes do not have 5′-3′ exonuclease activity. Additional information about thermophilic nucleic acid polymerases may be found in (Southworth M W, et al. PNAS. 1996; 93(11):5281-5285; Bergen K, et al. ChemBioChem. 2013; 14(9): 1058-1062; Kumar S, et al. Scientific Reports. 2012; 2:684; Fuller C W, et al. 2016; 113(19):5233-5238; Guo J, et al. Proceedings of the National Academy of Sciences of the United States of America. 2008; 105(27):9145-9150), which are incorporated herein in their entirety for all purposes.

As used herein, the term “exonuclease activity” is used in accordance with its ordinary meaning in the art, and refers to the removal of a nucleotide from a nucleic acid by an enzyme (e.g. DNA polymerase, a lambda exonuclease, Exo I, Exo III, T5, Exo V, Exo VII or the like). For example, during polymerization, nucleotides are added to the 3′ end of the primer strand. Occasionally a DNA polymerase incorporates an incorrect nucleotide to the 3′-OH terminus of the primer strand, wherein the incorrect nucleotide cannot form a hydrogen bond to the corresponding base in the template strand. Such a nucleotide, added in error, is removed from the primer as a result of the 3′ to 5′ exonuclease activity of the DNA polymerase. In embodiments, exonuclease activity may be referred to as “proofreading.” When referring to 3′-5′ exonuclease activity, it is understood that the DNA polymerase facilitates a hydrolyzing reaction that breaks phosphodiester bonds at the 3′ end of a polynucleotide chain to excise the nucleotide. In embodiments, 3′-5′ exonuclease activity refers to the successive removal of nucleotides in single-stranded DNA in a 3′->5′ direction, releasing deoxyribonucleoside 5′-monophosphates one after another. Methods for quantifying exonuclease activity are known in the art, see for example Southworth et al, PNAS Vol 93, 8281-8285 (1996). In embodiments, 5′-3′ exonuclease activity refers to the successive removal of nucleotides in double-stranded DNA in a 5′->3′ direction. In embodiments, the 5′-3′ exonuclease is lambda exonuclease. For example, lambda exonuclease catalyzes the removal of 5′ mononucleotides from duplex DNA, with a preference for 5′ phosphorylated double-stranded DNA. In other embodiments, the 5′-3′ exonuclease is E. coli DNA Polymerase I.

As used herein, the term “incorporating” or “chemically incorporating,” when used in reference to a primer and cognate nucleotide, refers to the process of joining the cognate nucleotide to the primer or extension product thereof by formation of a phosphodiester bond.

As used herein, the term “selective” or “selectivity” or the like of a compound refers to the compound's ability to discriminate between molecular targets. For example, a chemical reagent may selectively modify one nucleotide type in that it reacts with one nucleotide type (e.g., cytosines) and not other nucleotide types (e.g., adenine, thymine, or guanine). When used in the context of sequencing, such as in “selectively sequencing,” this term refers to sequencing one or more target polynucleotides from an original starting population of polynucleotides, and not sequencing non-target polynucleotides from the starting population. Typically, selectively sequencing one or more target polynucleotides involves differentially manipulating the target polynucleotides based on known sequence. For example, target polynucleotides may be hybridized to a probe oligonucleotide that may be labeled (such as with a member of a binding pair) or bound to a surface. In embodiments, hybridizing a target polynucleotide to a probe oligonucleotide includes the step of displacing one strand of a double-stranded nucleic acid. Probe-hybridized target polynucleotides may then be separated from non-hybridized polynucleotides, such as by removing probe-bound polynucleotides from the starting population or by washing away polynucleotides that are not bound to a probe. The result is a selected subset of the starting population of polynucleotides, which is then subjected to sequencing, thereby selectively sequencing the one or more target polynucleotides.

As used herein, the term “template polynucleotide” refers to any polynucleotide molecule that may be bound by a polymerase and utilized as a template for nucleic acid synthesis. A template polynucleotide may be a target polynucleotide. In general, the term “target polynucleotide” refers to a nucleic acid molecule or polynucleotide in a starting population of nucleic acid molecules having a target sequence whose presence, amount, and/or nucleotide sequence, or changes in one or more of these, are desired to be determined. The target sequence may be a portion of a gene, a regulatory sequence, genomic DNA, cDNA, RNA including mRNA, miRNA, rRNA, or others. The target sequence may be a target sequence from a sample or a secondary target such as a product of an amplification reaction. A target polynucleotide is not necessarily any single molecule or sequence. For example, a target polynucleotide may be any one of a plurality of target polynucleotides in a reaction, or all polynucleotides in a given reaction, depending on the reaction conditions. For example, in a nucleic acid amplification reaction with random primers, all polynucleotides in a reaction may be amplified. As a further example, a collection of targets may be simultaneously assayed using polynucleotide primers directed to a plurality of targets in a single reaction. As yet another example, all or a subset of polynucleotides in a sample may be modified by the addition of a primer-binding sequence (such as by the ligation of adapters containing the primer binding sequence), rendering each modified polynucleotide a target polynucleotide in a reaction with the corresponding primer polynucleotide(s). In embodiments, the template polynucleotide includes a target nucleic acid sequence and one or more barcode sequences. In embodiments, the template polynucleotide is a barcode sequence.

In embodiments, a target polynucleotide is a cell-free polynucleotide. In general, the terms “cell-free,” “circulating,” and “extracellular” as applied to polynucleotides (e.g. “cell-free DNA” (cfDNA) and “cell-free RNA” (cfRNA)) are used interchangeably to refer to polynucleotides present in a sample from a subject or portion thereof that can be isolated or otherwise manipulated without applying a lysis step to the sample as originally collected (e.g., as in extraction from cells or viruses). Cell-free polynucleotides are thus unencapsulated or “free” from the cells or viruses from which they originate, even before a sample of the subject is collected. Cell-free polynucleotides may be produced as a byproduct of cell death (e.g., apoptosis or necrosis) or cell shedding, releasing polynucleotides into surrounding body fluids or into circulation. Accordingly, cell-free polynucleotides may be isolated from a non-cellular fraction of blood (e.g., serum or plasma), from other bodily fluids (e.g., urine), or from non-cellular fractions of other types of samples.

As used herein, the terms “specific”, “specifically”, “specificity”, or the like of a compound refers to the compound's ability to cause a particular action, such as binding, to a particular molecular target with minimal or no action to other proteins in the cell.

The terms “attached,” “bind,” and “bound” as used herein are used in accordance with their plain and ordinary meanings and refer to an association between atoms or molecules. The association can be direct or indirect. For example, attached molecules may be directly bound to one another, e.g., by a covalent bond or non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). As a further example, two molecules may be bound indirectly to one another by way of direct binding to one or more intermediate molecules, thereby forming a complex.

“Specific binding” is where the binding is selective between two molecules. A particular example of specific binding is that which occurs between an antibody and an antigen. Typically, specific binding can be distinguished from non-specific when the dissociation constant (KD) is less than about 1×10⁻⁵ M or less than about 1×10⁻⁶ M or 1×10⁻⁷ M. Specific binding can be detected, for example, by ELISA, immunoprecipitation, coprecipitation, with or without chemical crosslinking, two-hybrid assays and the like. In embodiments, the KD (equilibrium dissociation constant) between two specific binding molecules is less than 10⁻⁶ M, less than 10⁻⁷ M, less than 10⁻⁸ M, less than 10⁻⁹ M, less than 10⁻⁹ M, less than 10⁻¹¹ M, or less than about 10⁻¹² M or less.

As used herein, the terms “sequencing”, “sequence determination”, “determining a nucleotide sequence”, and the like include determination of a partial or complete sequence information (e.g., a sequence) of a polynucleotide being sequenced, and particularly physical processes for generating such sequence information. That is, the term includes sequence comparisons, consensus sequence determination, contig assembly, fingerprinting, and like levels of information about a target polynucleotide, as well as the express identification and ordering of nucleotides in a target polynucleotide. The term also includes the determination of the identification, ordering, and locations of one, two, or three of the four types of nucleotides within a target polynucleotide. In some embodiments, a sequencing process described herein includes contacting a template and an annealed primer with a suitable polymerase under conditions suitable for polymerase extension and/or sequencing.

As used herein, the term “polymer” refers to macromolecules having one or more structurally unique repeating units. The repeating units are referred to as “monomers,” which are polymerized for the polymer. Typically, a polymer is formed by monomers linked in a chain-like structure. A polymer formed entirely from a single type of monomer is referred to as a “homopolymer.” A polymer formed from two or more unique repeating structural units may be referred to as a “copolymer.” A polymer may be linear or branched, and may be random, block, polymer brush, hyperbranched polymer, bottlebrush polymer, dendritic polymer, or polymer micelles. The term “polymer” includes homopolymers, copolymers, tripolymers, tetra polymers and other polymeric molecules made from monomeric subunits. Copolymers include alternating copolymers, periodic copolymers, statistical copolymers, random copolymers, block copolymers, linear copolymers and branched copolymers. The term “polymerizable monomer” is used in accordance with its meaning in the art of polymer chemistry and refers to a compound that may covalently bind chemically to other monomer molecules (such as other polymerizable monomers that are the same or different) to form a polymer.

Polymers can be hydrophilic, hydrophobic or amphiphilic, as known in the art. Thus, “hydrophilic polymers” are substantially miscible with water and include, but are not limited to, polyethylene glycol and the like. “Hydrophobic polymers” are substantially immiscible with water and include, but are not limited to, polyethylene, polypropylene, polybutadiene, polystyrene, polymers disclosed herein, and the like. “Amphiphilic polymers” have both hydrophilic and hydrophobic properties and are typically copolymers having hydrophilic segment(s) and hydrophobic segment(s). Polymers include homopolymers, random copolymers, and block copolymers, as known in the art. The term “homopolymer” refers, in the usual and customary sense, to a polymer having a single monomeric unit. The term “copolymer” refers to a polymer derived from two or more monomeric species. The term “random copolymer” refers to a polymer derived from two or more monomeric species with no preferred ordering of the monomeric species. The term “block copolymer” refers to polymers having two or homopolymer subunits linked by covalent bond. Thus, the term “hydrophobic homopolymer” refers to a homopolymer which is hydrophobic. The term “hydrophobic block copolymer” refers to two or more homopolymer subunits linked by covalent bonds and which is hydrophobic.

As used herein, the term “hydrogel” refers to a three-dimensional polymeric structure that is substantially insoluble in water, but which is capable of absorbing and retaining large quantities of water to form a substantially stable, often soft and pliable, structure. In embodiments, water can penetrate in between polymer chains of a polymer network, subsequently causing swelling and the formation of a hydrogel. In embodiments, hydrogels are super-absorbent (e.g., containing more than about 90% water) and can be comprised of natural or synthetic polymers.

As used herein, the term “substrate” refers to a solid support material. The substrate can be non-porous or porous. The substrate can be rigid or flexible. As used herein, the terms “solid support” and “solid surface” refers to discrete solid or semi-solid surface. A solid support may encompass any type of solid, porous, or hollow sphere, ball, cylinder, or other similar configuration composed of plastic, ceramic, metal, or polymeric material (e.g., hydrogel) onto which a nucleic acid may be immobilized (e.g., covalently or non-covalently). A nonporous substrate generally provides a seal against bulk flow of liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefin copolymers, polyimides etc.), nylon, ceramics, resins, Zeonor, silica or silica-based materials including silicon and modified silicon, carbon, metals, inorganic glasses, optical fiber bundles, photopatternable dry film resists, UV-cured adhesives and polymers. Particularly useful solid supports for some embodiments have at least one surface located within a flow cell. Solid surfaces can also be varied in their shape depending on the application in a method described herein. For example, a solid surface useful herein can be planar, or contain regions which are concave or convex. In embodiments, the geometry of the concave or convex regions (e.g., wells) of the solid surface conform to the size and shape of the particle to maximize the contact between as substantially circular particle. In embodiments, the wells of an array are randomly located such that nearest neighbor features have random spacing between each other. Alternatively, in embodiments the spacing between the wells can be ordered, for example, forming a regular pattern. The term solid substrate is encompassing of a substrate (e.g., a flow cell) having a surface including a polymer coating covalently attached thereto. In embodiments, the solid substrate is a flow cell. The term “flow cell” as used herein refers to a chamber including a solid surface across which one or more fluid reagents can be flowed. Examples of flow cells and related fluidic systems and detection platforms that can be readily used in the methods of the present disclosure are described, for example, in Bentley et al., Nature 456:53-59 (2008). In certain embodiments a substrate includes a surface (e.g., a surface of a flow cell, a surface of a tube, a surface of a chip), for example a metal surface (e.g., steel, gold, silver, aluminum, silicon and copper). In embodiments a substrate (e.g., a substrate surface) is coated and/or includes functional groups and/or inert materials. In certain embodiments a substrate includes a bead, a chip, a capillary, a plate, a membrane, a wafer (e.g., silicon wafers), a comb, or a pin for example. In some embodiments a substrate includes a bead and/or a nanoparticle. A substrate can be made of a suitable material, non-limiting examples of which include a plastic or a suitable polymer (e.g., polycarbonate, poly(vinyl alcohol), poly(divinylbenzene), polystyrene, polyamide, polyester, polyvinylidene difluoride (PVDF), polyethylene, polyurethane, polypropylene, and the like), borosilicate, glass, nylon, Wang resin, Merrifield resin, metal (e.g., iron, a metal alloy, sepharose, agarose, polyacrylamide, dextran, cellulose and the like or combinations thereof. In embodiments a substrate includes a magnetic material (e.g., iron, nickel, cobalt, platinum, aluminum, and the like). In embodiments a substrate includes a magnetic bead (e.g., DYNABEADS®, hematite, AMPure XP). Magnets can be used to purify and/or capture nucleic acids bound to certain substrates (e.g., substrates including a metal or magnetic material). The flow cell is typically a glass slide containing small fluidic channels (e.g., a glass slide 75 mm×25 mm×1 mm having one or more channels), through which sequencing solutions (e.g., polymerases, nucleotides, and buffers) may traverse. Though typically glass, suitable flow cell materials may include polymeric materials, plastics, silicon, quartz (fused silica), Borofloat® glass, silica, silica-based materials, carbon, metals, an optical fiber or optical fiber bundles, sapphire, or plastic materials such as COCs and epoxies. The particular material can be selected based on properties desired for a particular use. For example, materials that are transparent to a desired wavelength of radiation are useful for analytical techniques that will utilize radiation of the desired wavelength. Conversely, it may be desirable to select a material that does not pass radiation of a certain wavelength (e.g., being opaque, absorptive, or reflective). In embodiments, the material of the flow cell is selected due to the ability to conduct thermal energy. In embodiments, a flow cell includes inlet and outlet ports and a flow channel extending there between.

The term “surface” is intended to mean an external part or external layer of a substrate. The surface can be in contact with another material such as a gas, liquid, gel, polymer, organic polymer, second surface of a similar or different material, metal, or coat. The surface, or regions thereof, can be substantially flat. The substrate and/or the surface can have surface features such as wells, pits, channels, ridges, raised regions, pegs, posts or the like.

The term “microplate”, or “multiwell container” as used herein, refers to a substrate including a surface, the surface including a plurality of reaction chambers separated from each other by interstitial regions on the surface. In embodiments, the microplate has dimensions as provided and described by American National Standards Institute (ANSI) and Society for Laboratory Automation And Screening (SLAS); for example the tolerances and dimensions set forth in ANSI SLAS 1-2004 (R2012); ANSI SLAS 2-2004 (R2012); ANSI SLAS 3-2004 (R2012); ANSI SLAS 4-2004 (R2012); and ANSI SLAS 6-2012, which are incorporated herein by reference. The dimensions of the microplate as described herein and the arrangement of the reaction chambers may be compatible with an established format for automated laboratory equipment. In embodiments, the device described herein provides methods for high-throughput screening. High-throughput screening (HTS) refers to a process that uses a combination of modern robotics, data processing and control software, liquid handling devices, and/or sensitive detectors, to efficiently process a large amount of (e.g., thousands, hundreds of thousands, or millions) samples in biochemical, genetic, or pharmacological experiments, either in parallel or in sequence, within a reasonably short period of time (e.g., days). Preferably, the process is amenable to automation, such as robotic simultaneous handling of 96 samples, 384 samples, 1536 samples or more. A typical HTS robot tests up to 100,000 to a few hundred thousand compounds per day. The samples are often in small volumes, such as no more than 1 mL, 500 μl, 200 μl, 100 μl, 50 μl or less. Through this process, one can rapidly identify active compounds, small molecules, antibodies, proteins or polynucleotides in a cell.

The reaction chambers may be provided as wells of a multiwell container (alternatively referred to as reaction chambers), for example a microplate may contain 2, 4, 6, 12, 24, 48, 96, 384, or 1536 sample wells. In embodiments, the 96 and 384 wells are arranged in a 2:3 rectangular matrix. In embodiments, the 24 wells are arranged in a 3:8 rectangular matrix. In embodiments, the 48 wells are arranged in a 3:4 rectangular matrix. In embodiments, the reaction chamber is a microscope slide (e.g., a glass slide about 75 mm by about 25 mm). In embodiments the slide is a concavity slide (e.g., the slide includes a depression). In embodiments, the slide includes a coating for enhanced cell adhesion (e.g., poly-L-lysine, silanes, carbon nanotubes, polymers, epoxy resins, or gold). In embodiments, the microplate is about 5 inches by about 3.33 inches, and includes a plurality of 5 mm diameter wells. In embodiments, the microplate is about 5 inches by about 3.33 inches, and includes a plurality of 6 mm diameter wells. In embodiments, the microplate is about 5 inches by about 3.33 inches, and includes a plurality of 7 mm diameter wells. In embodiments, the microplate is about 5 inches by about 3.33 inches, and includes a plurality of 7.5 mm diameter wells. In embodiments, the microplate is 5 inches by 3.33 inches, and includes a plurality of 7.5 mm diameter wells. In embodiments, the microplate is about 5 inches by about 3.33 inches, and includes a plurality of 8 mm diameter wells. In embodiments, the microplate is a flat glass or plastic tray in which an array of wells are formed, wherein each well can hold between from a few microliters to hundreds of microliters of fluid reagents and samples. In embodiments, the microplate has a rectangular shape that measures 127.7 mm+0.5 mm in length by 85.4 mm±0.5 mm in width, and includes 6, 12, 24, 48, or 96 wells, wherein each well has an average diameter of about 5-7 mm. In embodiments, the microplate has a rectangular shape that measures 127.7 mm±0.5 mm in length by 85.4 mm±0.5 mm in width, and includes 6, 12, 24, 48, or 96 wells, wherein each well has an average diameter of about 6 mm.

The term “well” refers to a discrete concave feature in a substrate having a surface opening that is completely surrounded by interstitial region(s) of the surface. Wells can have any of a variety of shapes at their opening in a surface including but not limited to round, elliptical, square, polygonal, or star shaped (i.e., star shaped with any number of vertices). The cross section of a well taken orthogonally with the surface may be curved, square, polygonal, hyperbolic, conical, or angular. The wells of a microplate are available in different shapes, for example F-Bottom: flat bottom; C-Bottom: bottom with minimal rounded edges; V-Bottom: V-shaped bottom; or U-Bottom: U-shaped bottom. In embodiments, the well is substantially square. In embodiments, the well is square. In embodiments, the well is F-bottom. In embodiments, the microplate includes 24 substantially round flat bottom wells. In embodiments, the microplate includes 48 substantially round flat bottom wells. In embodiments, the microplate includes 96 substantially round flat bottom wells. In embodiments, the microplate includes 384 substantially square flat bottom wells.

The discrete regions (i.e., features, wells) of the microplate may have defined locations in a regular array, which may correspond to a rectilinear pattern, circular pattern, hexagonal pattern, or the like. In embodiments, the pattern of wells includes concentric circles of regions, spiral patterns, rectilinear patterns, hexagonal patterns, and the like. In embodiments, the pattern of wells is arranged in a rectilinear or hexagonal pattern A regular array of such regions is advantageous for detection and data analysis of signals collected from the arrays during an analysis. These discrete regions are separated by interstitial regions. As used herein, the term “interstitial region” refers to an area in a substrate or on a surface that separates other areas of the substrate or surface. For example, an interstitial region can separate one concave feature of an array from another concave feature of the array. The two regions that are separated from each other can be discrete, lacking contact with each other. In another example, an interstitial region can separate a first portion of a feature from a second portion of a feature. In embodiments the interstitial region is continuous whereas the features are discrete, for example, as is the case for an array of wells in an otherwise continuous surface. The separation provided by an interstitial region can be partial or full separation. In embodiments, interstitial regions have a surface material that differs from the surface material of the wells (e.g., the interstitial region contains a photoresist and the surface of the well is glass). In embodiments, interstitial regions have a surface material that is the same as the surface material of the wells (e.g., both the surface of the interstitial region and the surface of well contain a polymer or copolymer).

As used herein, the term “sequencing reaction mixture” is used in accordance with its plain and ordinary meaning and refers to an aqueous mixture that contains the reagents necessary to allow dNTP or dNTP analogue (e.g., a modified nucleotide) to add a nucleotide to a DNA strand by a DNA polymerase. In embodiments, the sequencing reaction mixture includes a buffer. In embodiments, the buffer includes an acetate buffer, 3-(N-morpholino)propanesulfonic acid (MOPS) buffer, N-(2-Acetamido)-2-aminoethanesulfonic acid (ACES) buffer, phosphate-buffered saline (PBS) buffer, 4-(2-hydroxyethyl)-1-piperazineethanesulfonic acid (HEPES) buffer, N-(1,1-Dimethyl-2-hydroxyethyl)-3-amino-2-hydroxypropanesulfonic acid (AMPSO) buffer, borate buffer (e.g., borate buffered saline, sodium borate buffer, boric acid buffer), 2-Amino-2-methyl-1,3-propanediol (AMPD) buffer, N-cyclohexyl-2-hydroxyl-3-aminopropanesulfonic acid (CAPSO) buffer, 2-Amino-2-methyl-1-propanol (AMP) buffer, 4-(cyclohexylamino)-1-butanesulfonic acid (CABS) buffer, glycine-NaOH buffer, N-Cyclohexyl-2-aminocthanesulfonic acid (CHES) buffer, tris(hydroxymethyl)aminomethane (Tris) buffer, or a N-cyclohexyl-3-aminopropanesulfonic acid (CAPS) buffer. In embodiments, the buffer is a borate buffer. In embodiments, the buffer is a CHES buffer. In embodiments, the sequencing reaction mixture includes nucleotides, wherein the nucleotides include a reversible terminating moiety and a label covalently linked to the nucleotide via a cleavable linker. In embodiments, the sequencing reaction mixture includes a buffer, DNA polymerase, detergent (e.g., Triton X), a chelator (e.g., EDTA), and/or salts (e.g., ammonium sulfate, magnesium chloride, sodium chloride, or potassium chloride).

As used herein, the term “sequencing cycle” is used in accordance with its plain and ordinary meaning and refers to incorporating one or more nucleotides (e.g., nucleotide analogues) to the 3′ end of a polynucleotide with a polymerase, and detecting one or more labels that identify the one or more nucleotides incorporated. In embodiments, one nucleotide (e.g., a modified nucleotide) is incorporated per sequencing cycle. The sequencing may be accomplished by, for example, sequencing by synthesis, pyrosequencing, and the like. In embodiments, a sequencing cycle includes extending a complementary polynucleotide by incorporating a first nucleotide using a polymerase, wherein the polynucleotide is hybridized to a template nucleic acid, detecting the first nucleotide, and identifying the first nucleotide. In embodiments, to begin a sequencing cycle, one or more differently labeled nucleotides and a DNA polymerase can be introduced. Following nucleotide addition, signals produced (e.g., via excitation and emission of a detectable label) can be detected to determine the identity of the incorporated nucleotide (based on the labels on the nucleotides). Reagents can then be added to remove the 3′ reversible terminator and to remove labels from each incorporated base. Reagents, enzymes, and other substances can be removed between steps by washing. Cycles may include repeating these steps, and the sequence of each cluster is read over the multiple repetitions.

As used herein, the term “extension” or “elongation” is used in accordance with their plain and ordinary meanings and refer to synthesis by a polymerase of a new polynucleotide strand complementary to a template strand by adding free nucleotides (e.g., dNTPs) from a reaction mixture that are complementary to the template in the 5′-to-3′ direction. Extension includes condensing the 5′-phosphate group of the dNTPs with the 3′-hydroxy group at the end of the nascent (elongating) DNA strand.

As used herein, the term “sequencing read” is used in accordance with its plain and ordinary meaning and refers to an inferred sequence of nucleotide bases (or nucleotide base probabilities) corresponding to all or part of a single polynucleotide fragment. A sequencing read may include 10, 20, 30, 40, 50, 60, 70, 80, 90, 100, 150, 200, 250, or more nucleotide bases. In embodiments, a sequencing read includes reading a barcode sequence and a template nucleotide sequence. In embodiments, a sequencing read includes reading a template nucleotide sequence. In embodiments, a sequencing read includes reading a barcode and not a template nucleotide sequence. Reads of length 20-40 base pairs (bp) are referred to as ultra-short. Typical sequencers produce read lengths in the range of 100-500 bp. Read length is a factor which can affect the results of biological studies. For example, longer read lengths improve the resolution of de novo genome assembly and detection of structural variants. In embodiments, a sequencing read includes reading a barcode and a template nucleotide sequence. In embodiments, a sequencing read includes reading a template nucleotide sequence. In embodiments, a sequencing read includes reading a barcode and not a template nucleotide sequence. In embodiments, a sequencing read includes a computationally derived string corresponding to the detected label. In some embodiments, a sequencing read may include 300, 400, 500, 600, 700, 800, 900, 1,000, 1,100, 1,200, 1,300, 1,400, 1,500, or more nucleotide bases.

The term “multiplexing” as used herein refers to an analytical method in which the presence and/or amount of multiple targets, e.g., multiple nucleic acid target sequences, can be assayed simultaneously by using the methods and devices as described herein, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic. As used herein, the term “multiplex” is used to refer to an assay in which multiple (i.e. at least two) different biomolecules are assayed at the same time, and more particularly in the same aliquot of the sample, or in the same reaction mixture. In embodiments, more than two different biomolecules are assayed at the same time. In embodiments, at least 2, 4, 6, 8, 10, 20, 50, 100, 200, 300, 400, 500, 600, 700, 800, 900, 1000, 1100, 1200, 1300, 1400 or 1500 or more biomolecules are detected according to the present method.

Complementary single stranded nucleic acids and/or substantially complementary single stranded nucleic acids can hybridize to each other under hybridization conditions, thereby forming a nucleic acid that is partially or fully double stranded. All or a portion of a nucleic acid sequence may be substantially complementary to another nucleic acid sequence, in some embodiments. As referred to herein, “substantially complementary” refers to nucleotide sequences that can hybridize with each other under suitable hybridization conditions. Hybridization conditions can be altered to tolerate varying amounts of sequence mismatch within complementary nucleic acids that are substantially complementary. Substantially complementary portions of nucleic acids that can hybridize to each other can be 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other. In some embodiments substantially complementary portions of nucleic acids that can hybridize to each other are 100% complementary. Nucleic acids, or portions thereof, that are configured to hybridize to each other often include nucleic acid sequences that are substantially complementary to each other.

“Hybridize” shall mean the annealing of a nucleic acid sequence to another nucleic acid sequence (e.g., one single-stranded nucleic acid (such as a primer) to another nucleic acid) based on the well-understood principle of sequence complementarity. In an embodiment the other nucleic acid is a single-stranded nucleic acid. In some embodiments, one portion of a nucleic acid hybridizes to itself, such as in the formation of a hairpin structure. The propensity for hybridization between nucleic acids depends on the temperature and ionic strength of their milieu, the length of the nucleic acids and the degree of complementarity. The effect of these parameters on hybridization is described in, for example, Sambrook J., Fritsch E. F., Maniatis T., Molecular cloning: a laboratory manual, Cold Spring Harbor Laboratory Press, New York (1989). As used herein, hybridization of a primer, or of a DNA extension product, respectively, is extendable by creation of a phosphodiester bond with an available nucleotide or nucleotide analogue capable of forming a phosphodiester bond, therewith. For example, hybridization can be performed at a temperature ranging from 15° C. to 95° C. In some embodiments, the hybridization is performed at a temperature of about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., or about 95° C. In other embodiments, the stringency of the hybridization can be further altered by the addition or removal of components of the buffered solution.

As used herein, “specifically hybridizes” refers to preferential hybridization under hybridization conditions where two nucleic acids, or portions thereof, that are substantially complementary, hybridize to each other and not to other nucleic acids that are not substantially complementary to either of the two nucleic acids. For example, specific hybridization includes the hybridization of a primer or capture nucleic acid to a portion of a target nucleic acid (e.g., a template, or adapter portion of a template) that is substantially complementary to the primer or capture nucleic acid. In some embodiments nucleic acids, or portions thereof, that are configured to specifically hybridize are often about 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more or 100% complementary to each other over a contiguous portion of nucleic acid sequence. A specific hybridization discriminates over non-specific hybridization interactions (e.g., two nucleic acids that a not configured to specifically hybridize, e.g., two nucleic acids that are 80% or less, 70% or less, 60% or less or 50% or less complementary) by about 2-fold or more, often about 10-fold or more, and sometimes about 100-fold or more, 1000-fold or more, 10,000-fold or more, 100,000-fold or more, or 1,000,000-fold or more. Two nucleic acid strands that are hybridized to each other can form a duplex which includes a double stranded portion of nucleic acid.

As used herein, the term “adjacent,” refers to two nucleotide sequences in a nucleic acid, can refer to nucleotide sequences separated by 0 to about 20 nucleotides, more specifically, in a range of about 1 to about 10 nucleotides, or to sequences that directly abut one another. As those of skill in the art appreciate, two nucleotide sequences that that are to ligated together will generally directly abut one another.

A nucleic acid can be amplified by a suitable method. The term “amplification,” “amplified” or “amplifying” as used herein refers to subjecting a target nucleic acid in a sample to a process that linearly or exponentially generates amplicon nucleic acids having the same or substantially the same (e.g., substantially identical) nucleotide sequence as the target nucleic acid, or segment thereof, and/or a complement thereof (which may be referred to herein as an “amplification product” or “amplification products”). In some embodiments an amplification reaction comprises a suitable thermal stable polymerase. Thermal stable polymerases are known and are stable for prolonged periods of time, at temperature greater than 80° C. when compared to common polymerases found in most mammals. In certain embodiments the term “amplification,” “amplified” or “amplifying” refers to a method that includes a polymerase chain reaction (PCR). Conditions conducive to amplification (i.e., amplification conditions) are known and often include at least a suitable polymerase, a suitable template, a suitable primer or set of primers, suitable nucleotides (e.g., dNTPs), a suitable buffer, and application of suitable annealing, hybridization and/or extension times and temperatures. In certain embodiments an amplified product (e.g., an amplicon) can contain one or more additional and/or different nucleotides than the template sequence, or portion thereof, from which the amplicon was generated (e.g., a primer can contain “extra” nucleotides (such as a 5′ portion that does not hybridize to the template), or one or more mismatched bases within a hybridizing portion of the primer).

As used herein, bridge-PCR (bPCR) amplification is a method for solid-phase amplification as exemplified by the disclosures of U.S. Pat. Nos. 5,641,658; 7,115,400; and U.S. Patent Publ. No. 2008/0009420, each of which is incorporated herein by reference in its entirety. Bridge-PCR involves repeated polymerase chain reaction cycles, cycling between denaturation, annealing, and extension conditions and enables controlled, spatially-localized, amplification, to generate amplification products (e.g., amplicons) immobilized on a solid support in order to form arrays comprised of colonies (or “clusters”) of immobilized nucleic acid molecule.

Amplification according to the present teachings encompasses any means by which at least a part of at least one target nucleic acid is reproduced, typically in a template-dependent manner, including without limitation, a broad range of techniques for amplifying nucleic acid sequences, either linearly or exponentially. Illustrative means for performing an amplifying step include ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), and the like, including multiplex versions and combinations thereof, for example but not limited to, OLA (oligonucleotide ligation assay)/PCR, PCR/OLA, LDR/PCR, PCR/PCR/LDR, PCR/LDR, LCR/PCR, PCR/LCR (also known as combined chain reaction-CCR), and the like. Descriptions of such techniques can be found in, among other sources, Ausbel et al.; PCR Primer: A Laboratory Manual, Diffenbach, Ed., Cold Spring Harbor Press (1995); The Electronic Protocol Book, Chang Bioscience (2002); Msuih et al., J. Clin. Micro. 34:501-07 (1996); The Nucleic Acid Protocols Handbook, R. Rapley, ed., Humana Press, Totowa, N.J. (2002); Abramson et al., Curr Opin Biotechnol. 1993 February; 4(1):41-7, U.S. Pat. Nos. 6,027,998; 6,605,451, Barany et al., PCT Publication No. WO 97/31256; Wenz et al., PCT Publication No. WO 01/92579; Day et al., Genomics, 29(1): 152-162 (1995), Ehrlich et al., Science 252:1643-50 (1991); Innis et al., PCR Protocols: A Guide to Methods and Applications, Academic Press (1990); Favis et al., Nature Biotechnology 18:561-64 (2000); and Rabenau et al., Infection 28:97-102 (2000); Belgrader, Barany, and Lubin, Development of a Multiplex Ligation Detection Reaction DNA Typing Assay, Sixth International Symposium on Human Identification, 1995 (available on the world wide web at: promega.com/geneticidproc/ussymp6proc/blegrad.html-); LCR Kit Instruction Manual, Cat. #200520, Rev. #050002, Stratagene, 2002; Barany, Proc. Natl. Acad. Sci. USA 88:188-93 (1991); Bi and Sambrook, Nucl. Acids Res. 25:2924-2951 (1997); Zirvi et al., Nucl. Acid Res. 27:e40i-viii (1999); Dean et al., Proc Natl Acad Sci USA 99:5261-66 (2002); Barany and Gelfand, Gene 109:1-11 (1991); Walker et al., Nucl. Acid Res. 20:1691-96 (1992); Polstra et al., BMC Inf. Dis. 2:18-(2002); Lage et al., Genome Res. 2003 February; 13(2):294-307, and Landegren et al., Science 241:1077-80 (1988), Demidov, V., Expert Rev Mol Diagn. 2002 November; 2(6):542-8, Cook et al., J Microbiol Methods. 2003 May; 53(2): 165-74, Schweitzer et al., Curr Opin Biotechnol. 2001 February; 12(1):21-7, U.S. Pat. Nos. 5,830,711, 6,027,889, 5,686,243, PCT Publication No. WO0056927A3, and PCT Publication No. WO9803673A1.

In some embodiments, amplification includes at least one cycle of the sequential procedures of: annealing at least one primer with complementary or substantially complementary sequences in at least one target nucleic acid; synthesizing at least one strand of nucleotides in a template-dependent manner using a polymerase; and denaturing the newly-formed nucleic acid duplex to separate the strands. The cycle may or may not be repeated. Amplification can include thermocycling or can be performed isothermally.

As used herein, the term “rolling circle amplification (RCA)” refers to a nucleic acid amplification reaction that amplifies a circular nucleic acid template (e.g., single-stranded DNA circles) via a rolling circle mechanism. Rolling circle amplification reaction is initiated by the hybridization of a primer to a circular, often single-stranded, nucleic acid template. The nucleic acid polymerase then extends the primer that is hybridized to the circular nucleic acid template by continuously progressing around the circular nucleic acid template to replicate the sequence of the nucleic acid template over and over again (rolling circle mechanism). The rolling circle amplification typically produces concatemers including tandem repeat units of the circular nucleic acid template sequence. The rolling circle amplification may be a linear RCA (LRCA), exhibiting linear amplification kinetics (e.g., RCA using a single specific primer), or may be an exponential RCA (ERCA) exhibiting exponential amplification kinetics. Rolling circle amplification may also be performed using multiple primers (multiply primed rolling circle amplification or MPRCA) leading to hyper-branched concatemers. For example, in a double-primed RCA, one primer may be complementary, as in the linear RCA, to the circular nucleic acid template, whereas the other may be complementary to the tandem repeat unit nucleic acid sequences of the RCA product. Consequently, the double-primed RCA may proceed as a chain reaction with exponential (geometric) amplification kinetics featuring a ramifying cascade of multiple-hybridization, primer-extension, and strand-displacement events involving both the primers. This often generates a discrete set of concatemeric, double-stranded nucleic acid amplification products. The rolling circle amplification may be performed in-vitro under isothermal conditions using a suitable nucleic acid polymerase such as Phi29 DNA polymerase. RCA may be performed by using any of the DNA polymerases that are known in the art (e.g., a Phi29 DNA polymerase, a Bst DNA polymerase, or SD polymerase).

A nucleic acid can be amplified by a thermocycling method or by an isothermal amplification method. In some embodiments a rolling circle amplification method is used. In some embodiments amplification takes place on a solid support (e.g., within a flow cell) where a nucleic acid, nucleic acid library or portion thereof is immobilized. In certain sequencing methods, a nucleic acid library is added to a flow cell and immobilized by hybridization to anchors under suitable conditions. This type of nucleic acid amplification is often referred to as solid phase amplification. In some embodiments of solid phase amplification, all or a portion of the amplified products are synthesized by an extension initiating from an immobilized primer. Solid phase amplification reactions are analogous to standard solution phase amplifications except that at least one of the amplification oligonucleotides (e.g., primers) is immobilized on a solid support.

In some embodiments solid phase amplification includes a nucleic acid amplification reaction including only one species of oligonucleotide primer immobilized to a surface or substrate. In certain embodiments solid phase amplification includes a plurality of different immobilized oligonucleotide primer species. In some embodiments solid phase amplification may include a nucleic acid amplification reaction including one species of oligonucleotide primer immobilized on a solid surface and a second different oligonucleotide primer species in solution. Multiple different species of immobilized or solution-based primers can be used. Non-limiting examples of solid phase nucleic acid amplification reactions include interfacial amplification, bridge PCR amplification, emulsion PCR, WildFire amplification (e.g., US patent publication US20130012399), the like or combinations thereof.

As used herein, the terms “cluster” and “colony” are used interchangeably to refer to a discrete site on a solid support that includes a plurality of immobilized polynucleotides and a plurality of immobilized complementary polynucleotides. The term “clustered array” refers to an array formed from such clusters or colonies. In this context the term “array” is not to be understood as requiring an ordered arrangement of clusters. The term “array” is used in accordance with its ordinary meaning in the art, and refers to a population of different molecules that are attached to one or more solid-phase substrates such that the different molecules can be differentiated from each other according to their relative location. An array can include different molecules that are each located at different addressable features on a solid-phase substrate. The molecules of the array can be nucleic acid primers, nucleic acid probes, nucleic acid templates or nucleic acid enzymes such as polymerases or ligases. Arrays useful in the invention can have densities that ranges from about 2 different features to many millions, billions or higher. The density of an array can be from 2 to as many as a billion or more different features per square cm. For example an array can have at least about 100 features/cm², at least about 1,000 features/cm², at least about 10,000 features/cm², at least about 100,000 features/cm², at least about 10,000,000 features/cm², at least about 100,000,000 features/cm², at least about 1,000,000,000 features/cm², at least about 2,000,000,000 features/cm² or higher. In embodiments, the arrays have features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher.

Provided herein are methods, systems, and compositions for analyzing a sample (e.g., sequencing nucleic acids within a sample) in situ. The term “in situ” is used in accordance with its ordinary meaning in the art and refers to a sample surrounded by at least a portion of its native environment, such as may preserve the relative position of two or more elements. For example, an extracted human cell obtained is considered in situ when the cell is retained in its local microenvironment so as to avoid extracting the target (e.g., nucleic acid molecules or proteins) away from their native environment. An in situ sample (e.g., a cell) can be obtained from a suitable subject. An in situ cell sample may refer to a cell and its surrounding milieu, or a tissue. A sample can be isolated or obtained directly from a subject or part thereof. In embodiments, the methods described herein (e.g., sequencing a plurality of target nucleic acids of a cell in situ) are applied to an isolated cell (i.e., a cell not surrounded by least a portion of its native environment). For the avoidance of any doubt, when the method is performed within a cell (e.g., an isolated cell) the method may be considered in situ. In some embodiments, a sample is obtained indirectly from an individual or medical professional. A sample can be any specimen that is isolated or obtained from a subject or part thereof. A sample can be any specimen that is isolated or obtained from multiple subjects. Non-limiting examples of specimens include fluid or tissue from a subject, including, without limitation, blood or a blood product (e.g., serum, plasma, platelets, buffy coats, or the like), umbilical cord blood, chorionic villi, amniotic fluid, cerebrospinal fluid, spinal fluid, lavage fluid (e.g., lung, gastric, peritoneal, ductal, car, arthroscopic), a biopsy sample, celocentesis sample, cells (blood cells, lymphocytes, placental cells, stem cells, bone marrow derived cells, embryo or fetal cells) or parts thereof (e.g., mitochondrial, nucleus, extracts, or the like), urine, feces, sputum, saliva, nasal mucous, prostate fluid, lavage, semen, lymphatic fluid, bile, tears, sweat, breast milk, breast fluid, the like or combinations thereof. Non-limiting examples of tissues include organ tissues (e.g., liver, kidney, lung, thymus, adrenals, skin, bladder, reproductive organs, intestine, colon, spleen, brain, the like or parts thereof), epithelial tissue, hair, hair follicles, ducts, canals, bone, eye, nose, mouth, throat, car, nails, the like, parts thereof or combinations thereof. A sample may include cells or tissues that are normal, healthy, diseased (e.g., infected), and/or cancerous (e.g., cancer cells). A sample obtained from a subject may include cells or cellular material (e.g., nucleic acids) of multiple organisms (e.g., virus nucleic acid, fetal nucleic acid, bacterial nucleic acid, parasite nucleic acid). A sample may include a cell and RNA transcripts. A sample can include nucleic acids obtained from one or more subjects. In some embodiments a sample includes nucleic acid obtained from a single subject. A subject can be any living or non-living organism, including but not limited to a human, non-human animal, plant, bacterium, fungus, virus, or protist. A subject may be any age (e.g., an embryo, a fetus, infant, child, adult). A subject can be of any sex (e.g., male, female, or combination thereof). A subject may be pregnant. In some embodiments, a subject is a mammal. In some embodiments, a subject is a plant. In some embodiments, a subject is a human subject. A subject can be a patient (e.g., a human patient). In some embodiments a subject is suspected of having a genetic variation or a disease or condition associated with a genetic variation.

As used herein, the term “disease state” is used in accordance with its plain and ordinary meaning and refers to any abnormal biological or aberrant state of a cell. The presence of a disease state may be identified by the same collection of biological constituents used to determine the cell's biological state. In general, a disease state will be detrimental to a biological system. A disease state may be a consequence of, inter alia, an environmental pathogen, for example a viral infection (e.g., HIV/AIDS, hepatitis B, hepatitis C, influenza, measles, etc.), a bacterial infection, a parasitic infection, a fungal infection, or infection by some other organism. A disease state may also be the consequence of some other environmental agent, such as a chemical toxin or a chemical carcinogen. As used herein, a disease state further includes genetic disorders wherein one or more copies of a gene is altered or disrupted, thereby affecting its biological function. Exemplary genetic diseases include, but are not limited to polycystic kidney disease, familial multiple endocrine neoplasia type I, neurofibromatoses, Tay-Sachs disease, Huntington's disease, sickle cell anemia, thalassemia, and Down's syndrome, as well as others (see, e.g., The Metabolic and Molecular Bases of Inherited Diseases, 7th ed., McGraw-Hill Inc., New York). Other exemplary diseases include, but are not limited to, cancer, hypertension, Alzheimer's disease, neurodegenerative diseases, and neuropsychiatric disorders such as bipolar affective disorders or paranoid schizophrenic disorders. Disease states are monitored to determine the level or severity (e.g., the stage or progression) of one or more disease states of a subject and, more specifically, detect changes in the biological state of a subject which are correlated to one or more disease states (see, e.g., U.S. Pat. No. 6,218,122, which is incorporated by reference herein in its entirety). In embodiments, methods provided herein are also applicable to monitoring the disease state or states of a subject undergoing one or more therapies. Thus, the present disclosure also provides, in some embodiments, methods for determining or monitoring efficacy of a therapy or therapies (i.e., determining a level of therapeutic effect) upon a subject. In embodiments, methods of the present disclosure can be used to assess therapeutic efficacy in a clinical trial, e.g., as an early surrogate marker for success or failure in such a clinical trial. Within eukaryotic cells, there are hundreds to thousands of signaling pathways that are interconnected. For this reason, perturbations in the function of proteins within a cell have numerous effects on other proteins and the transcription of other genes that are connected by primary, secondary, and sometimes tertiary pathways. This extensive interconnection between the function of various proteins means that the alteration of any one protein is likely to result in compensatory changes in a wide number of other proteins. In particular, the partial disruption of even a single protein within a cell, such as by exposure to a drug or by a disease state which modulates the gene copy number (e.g., a genetic mutation), results in characteristic compensatory changes in the transcription of enough other genes that these changes in transcripts can be used to define a “signature” of particular transcript alterations which are related to the disruption of function, e.g., a particular disease state or therapy, even at a stage where changes in protein activity are undetectable.

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymer. A protein may refer to a protein expressed in a cell.

A polypeptide, or a cell is “recombinant” when it is artificial or engineered, or derived from or contains an artificial or engineered protein or nucleic acid (e.g., non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

As used herein, a “single cell” refers to one cell. Single cells useful in the methods described herein can be obtained from a tissue of interest, or from a biopsy, blood sample, or cell culture. Additionally, cells from specific organs, tissues, tumors, neoplasms, or the like can be obtained and used in the methods described herein. In general, cells from any population can be used in the methods, such as a population of prokaryotic or eukaryotic organisms, including bacteria or yeast.

The term “cellular component” is used in accordance with its ordinary meaning in the art and refers to any organelle, nucleic acid, protein, or analyte that is found in a prokaryotic, eukaryotic, archaeal, or other organismic cell type. Examples of cellular components (e.g., a component of a cell) include RNA transcripts, proteins, membranes, lipids, and other analytes.

A “gene” refers to a polynucleotide that is capable of conferring biological function after being transcribed and/or translated.

As used herein, the terms “biomolecule” or “analyte” refer to an agent (e.g., a compound, macromolecule, or small molecule), and the like derived from a biological system (e.g., an organism, a cell, or a tissue). The biomolecule may contain multiple individual components that collectively construct the biomolecule, for example, in embodiments, the biomolecule is a polynucleotide wherein the polynucleotide is composed of nucleotide monomers. The biomolecule may be or may include DNA, RNA, organelles, carbohydrates, lipids, proteins, or any combination thereof. These components may be extracellular. In some examples, the biomolecule may be referred to as a clump or aggregate of combinations of components. In some instances, the biomolecule may include one or more constituents of a cell but may not include other constituents of the cell. In embodiments, a biomolecule is a molecule produced by a biological system (e.g., an organism). The biomolecule may be any substance (e.g. molecule) or entity that is desired to be detected by the method of the invention. The biomolecule is the “target” of the assay method of the invention. The biomolecule may accordingly be any compound that may be desired to be detected, for example a peptide or protein, or nucleic acid molecule or a small molecule, including organic and inorganic molecules. The biomolecule may be a cell or a microorganism, including a virus, or a fragment or product thereof. Biomolecules of particular interest may thus include proteinaceous molecules such as peptides, polypeptides, proteins or prions or any molecule which includes a protein or polypeptide component, etc., or fragments thereof. The biomolecule may be a single molecule or a complex that contains two or more molecular subunits, which may or may not be covalently bound to one another, and which may be the same or different. Thus, in addition to cells or microorganisms, such a complex biomolecule may also be a protein complex. Such a complex may thus be a homo- or hetero-multimer. Aggregates of molecules e.g., proteins may also be target analytes, for example aggregates of the same protein or different proteins. The biomolecule may also be a complex between proteins or peptides and nucleic acid molecules such as DNA or RNA. Of particular interest may be the interactions between proteins and nucleic acids, e.g., regulatory factors, such as transcription factors, and interactions between DNA or RNA molecules

As used herein, “biomaterial” refers to any biological material produced by an organism. In some embodiments, biomaterial includes secretions, extracellular matrix, proteins, lipids, organelles, membranes, cells, portions thereof, and combinations thereof. In some embodiments, cellular material includes secretions, extracellular matrix, proteins, lipids, organelles, membranes, cells, portions thereof, and combinations thereof. In some embodiments, biomaterial includes viruses. In some embodiments, the biomaterial is a replicating virus and thus includes virus infected cells. In embodiments, a biological sample includes biomaterials.

In some embodiments, a sample includes one or more nucleic acids, or fragments thereof. A sample can include nucleic acids obtained from one or more subjects. In some embodiments a sample includes nucleic acid obtained from a single subject. In some embodiments, a sample includes a mixture of nucleic acids. A mixture of nucleic acids can include two or more nucleic acid species having different nucleotide sequences, different fragment lengths, different origins (e.g., genomic origins, cell or tissue origins, subject origins, the like or combinations thereof), or combinations thereof. A sample may include synthetic nucleic acid.

A subject can be any living or non-living organism, including but not limited to a human, non-human animal, plant, bacterium, fungus, virus or protist. A subject may be any age (e.g., an embryo, a fetus, infant, child, adult). A subject can be of any sex (e.g., male, female, or combination thereof). A subject may be pregnant. In some embodiments, a subject is a mammal. In some embodiments, a subject is a human subject. A subject can be a patient (e.g., a human patient). In some embodiments a subject is suspected of having a genetic variation or a disease or condition associated with a genetic variation.

The methods and kits of the present disclosure may be applied, mutatis mutandis, to the sequencing of RNA, or to determining the identity of a ribonucleotide.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., packaging, buffers, written instructions for performing a method, etc.) from one location to another. For example, kits include one or more enclosures (e.g., boxes) containing the relevant reaction reagents and/or supporting materials. As used herein, the term “fragmented kit” refers to a delivery system including two or more separate containers that each contain a subportion of the total kit components. The containers may be delivered to the intended recipient together or separately. For example, a first container may contain an enzyme for use in an assay, while a second container contains oligonucleotides. In contrast, a “combined kit” refers to a delivery system containing all of the components of a reaction assay in a single container (e.g., in a single box housing each of the desired components). The term “kit” includes both fragmented and combined kits.

As used herein the term “determine” can be used to refer to the act of ascertaining, establishing or estimating. A determination can be probabilistic. For example, a determination can have an apparent likelihood of at least 50%, 75%, 90%, 95%, 98%, 99%, 99.9% or higher. In some cases, a determination can have an apparent likelihood of 100%. An exemplary determination is a maximum likelihood analysis or report. As used herein, the term “identify,” when used in reference to a thing, can be used to refer to recognition of the thing, distinction of the thing from at least one other thing or categorization of the thing with at least one other thing. The recognition, distinction or categorization can be probabilistic. For example, a thing can be identified with an apparent likelihood of at least 50%, 75%, 90%, 95%, 98%, 99%, 99.9% or higher. A thing can be identified based on a result of a maximum likelihood analysis. In some cases, a thing can be identified with an apparent likelihood of 100%.

The terms “bioconjugate group,” “bioconjugate reactive moiety,” and “bioconjugate reactive group” refer to a chemical moiety which participates in a reaction to form a bioconjugate linker (e.g., covalent linker). Non-limiting examples of bioconjugate reactive groups and the resulting bioconjugate reactive linkers may be found in the Bioconjugate Table below:

| Bioconjugate reactive | Bioconjugate reactive | |
|---|---|---|
| group 1 | group 2 | |
| (e.g., electrophilic | (e.g., nucleophilic | Resulting |
| bioconjugate reactive | bioconjugate | Bioconjugate |
| moiety) | reactive moiety) | reactive linker |
| activated esters | amines/anilines | carboxamides |
| acrylamides | thiols | thioethers |
| acyl azides | amines/anilines | carboxamides |
| acyl halides | amines/anilines | carboxamides |
| acyl halides | alcohols/phenols | esters |
| acyl nitriles | alcohols/phenols | esters |
| acyl nitriles | amines/anilines | carboxamides |
| aldehydes | amines/anilines | imines |
| aldehydes or ketones | hydrazines | hydrazones |
| aldehydes or ketones | hydroxylamines | oximes |
| alkyl halides | amines/anilines | alkyl amines |
| alkyl halides | carboxylic acids | esters |
| alkyl halides | thiols | thioethers |
| alkyl halides | alcohols/phenols | ethers |
| alkyl sulfonates | thiols | thioethers |
| alkyl sulfonates | carboxylic acids | esters |
| alkyl sulfonates | alcohols/phenols | ethers |
| anhydrides | alcohols/phenols | esters |
| anhydrides | amines/anilines | carboxamides |
| aryl halides | thiols | thiophenols |
| aryl halides | amines | aryl amines |
| aziridines | thiols | thioethers |
| boronates | glycols | boronate esters |
| carbodiimides | carboxylic acids | N-acylureas or |
| | | anhydrides |
| diazoalkanes | carboxylic acids | esters |
| epoxides | thiols | thioethers |
| haloacetamides | thiols | thioethers |
| haloplatinate | amino | platinum complex |
| haloplatinate | heterocycle | platinum complex |
| haloplatinate | thiol | platinum complex |
| halotriazines | amines/anilines | aminotriazines |
| halotriazines | alcohols/phenols | triazinyl ethers |
| halotriazines | thiols | triazinyl thioethers |
| imido esters | amines/anilines | amidines |
| isocyanates | amines/anilines | ureas |
| isocyanates | alcohols/phenols | urethanes |
| isothiocyanates | amines/anilines | thioureas |
| maleimides | thiols | thioethers |
| phosphoramidites | alcohols | phosphite esters |
| silyl halides | alcohols | silyl ethers |
| sulfonate esters | amines/anilines | alkyl amines |
| sulfonate esters | thiols | thioethers |
| sulfonate esters | carboxylic acids | esters |
| sulfonate esters | alcohols | ethers |
| sulfonyl halides | amines/anilines | sulfonamides |
| sulfonyl halides | phenols/alcohols | sulfonate esters |

As used herein, the term “bioconjugate reactive moiety” and “bioconjugate reactive group” refers to a moiety or group capable of forming a bioconjugate (e.g., covalent linker) as a result of the association between atoms or molecules of bioconjugate reactive groups. The association can be direct or indirect. For example, a conjugate between a first bioconjugate reactive group (e.g., —NH₂, —COOH, —N— hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

Useful bioconjugate reactive groups used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (c) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (l) metal silicon oxide bonding; (m) metal bonding to reactive phosphorus groups (e.g., phosphines) to form, for example, phosphate diester bonds; (n) azides coupled to alkynes using copper catalyzed cycloaddition click chemistry; (o) biotin conjugate can react with avidin or strepavidin to form a avidin-biotin complex or streptavidin-biotin complex.

An “antibody” (Ab) is a protein that binds specifically to a particular substance, known as an “antigen” (Ag). An “antibody” or “antigen-binding fragment” is an immunoglobulin that binds a specific “epitope.” The term encompasses polyclonal, monoclonal, and chimeric antibodies. In nature, antibodies are generally produced by lymphocytes in response to immune challenge, such as by infection or immunization. An “antigen” (Ag) is any substance that reacts specifically with antibodies or T lymphocytes (T cells). An antibody may include the entire antibody as well as any antibody fragments capable of binding the antigen or antigenic fragment of interest. Examples include complete antibody molecules, antibody fragments, such as Fab, F(ab′)2, CDRs, VL, VH, and any other portion of an antibody which is capable of specifically binding to an antigen. Antibodies used herein are immunospecific for, and therefore specifically and selectively bind to, for example, proteins either detected (e.g., biological targets of interest) or used for detection (e.g., probes containing oligonucleotide barcodes) in the methods and devices as described herein.

The term “covalent linker” is used in accordance with its ordinary meaning and refers to a divalent moiety which connects at least two moieties to form a molecule.

The term “non-covalent linker” is used in accordance with its ordinary meaning and refers to a divalent moiety which includes at least two molecules that are not covalently linked to each other but are capable of interacting with each other via a non-covalent bond (e.g., electrostatic interactions (e.g., ionic bond, hydrogen bond, halogen bond) or van der Waals interactions (e.g., dipole-dipole, dipole-induced dipole, London dispersion). In embodiments, the non-covalent linker is the result of two molecules that are not covalently linked to each other that interact with each other via a non-covalent bond.

As used herein a “genetically modifying agent” is a substance that alters the genetic sequence of a cell following exposure to the cell, resulting in an agent-mediated nucleic acid sequence. In embodiments, the genetically modifying agent is a small molecule, protein, pathogen (e.g., virus or bacterium), toxin, oligonucleotide, or antigen. In embodiments, the genetically modifying agent is a virus (e.g., influenza) and the agent-mediated nucleic acid sequence is the nucleic acid sequence that develops within a T-cell upon cellular exposure and contact with the virus. In embodiments, the genetically modifying agent modulates the expression of a nucleic acid sequence in a cell relative to a control (e.g., the absence of the genetically modifying agent).

Where a range of values is provided, it is understood that each intervening value, to the tenth of the unit of the lower limit unless the context clearly indicates otherwise, between the upper and lower limit of that range, and any other stated or unstated intervening value in, or smaller range of values within, that stated range is encompassed within the invention. The upper and lower limits of any such smaller range (within a more broadly recited range) may independently be included in the smaller ranges, or as particular values themselves, and are also encompassed within the invention, subject to any specifically excluded limit in the stated range. Where the stated range includes one or both of the limits, ranges excluding either or both of those included limits are also included in the invention.

As used herein, the term “upstream” refers to a region in the nucleic acid sequence that is towards the 5′ end of a particular reference point, and the term “downstream” refers to a region in the nucleic acid sequence that is toward the 3′ end of the reference point.

As used herein, the terms “incubate,” and “incubation refer collectively to altering the temperature of an object in a controlled manner such that conditions are sufficient for conducting the desired reaction. Thus, it is envisioned that the terms encompass heating a receptacle (e.g., a microplate) to a desired temperature and maintaining such temperature for a fixed time interval. Also included in the terms is the act of subjecting a receptacle to one or more heating and cooling cycles (i.e., “temperature cycling” or “thermal cycling”). While temperature cycling typically occurs at relatively high rates of change in temperature, the term is not limited thereto, and may encompass any rate of change in temperature.

As used herein, “biological activity” may include the in vivo activities of a compound or physiological responses that result upon in vivo administration of a compound, composition or other mixture. Biological activity, thus, may encompass therapeutic effects and pharmaceutical activity of such compounds, compositions and mixtures. Biological activities may be observed in vitro systems designed to test or use such activities.

The term “isolated” means altered or removed from the natural state. For example, a nucleic acid or a polypeptide naturally present in a living animal is not isolated, but the same nucleic acid or polypeptide partially or completely separated from the coexisting materials of its natural state is isolated. An isolated nucleic acid or protein can exist in substantially purified form, or can exist in a non-native environment such as, for example, a host cell. In embodiments, “isolated” refers to a nucleic acid, polynucleotide, polypeptide, protein, or other component that is partially or completely separated from components with which it is normally associated (other proteins, nucleic acids, cells, etc.).

The term “synthetic target” as used herein refers to a modified protein or nucleic acid such as those constructed by synthetic methods. In embodiments, a synthetic target is artificial or engineered, or derived from or contains an artificial or engineered protein or nucleic acid (e.g., non-natural or not wild type). For example, a polynucleotide that is inserted or removed such that it is not associated with nucleotide sequences that normally flank the polynucleotide as it is found in nature is a synthetic target polynucleotide.

The term “nucleic acid sequencing device” and the like means an integrated system of one or more chambers, ports, and channels that are interconnected and in fluid communication and designed for carrying out an analytical reaction or process, either alone or in cooperation with an appliance or instrument that provides support functions, such as sample introduction, fluid and/or reagent driving means, temperature control, detection systems, data collection and/or integration systems, for the purpose of determining the nucleic acid sequence of a template polynucleotide. Nucleic acid sequencing devices may further include valves, pumps, and specialized functional coatings on interior walls. Nucleic acid sequencing devices may include a receiving unit, or platen, that orients the flow cell such that a maximal surface area of the flow cell is available to be exposed to an optical lens. Other nucleic acid sequencing devices include those provided by Singular Genomics™ (e.g., the G4™ system), Illumina™ (e.g., HiSeq™, MiSeq™, NextSeq™, or NovaSeq™ systems), Life Technologies™ (e.g., ABI PRISM™, or SOLID™ systems), Pacific Biosciences (e.g., systems using SMRT™ Technology such as the Sequel™ or RS II™ systems), or Qiagen (e.g., Genereader™ system). Nucleic acid sequencing devices may further include fluidic reservoirs (e.g., bottles), valves, pressure sources, pumps, sensors, control systems, valves, pumps, and specialized functional coatings on interior walls. In embodiments, the device includes a plurality of a sequencing reagent reservoirs and a plurality of clustering reagent reservoirs. In embodiments, the clustering reagent reservoir includes amplification reagents (e.g., an aqueous buffer containing enzymes, salts, and nucleotides, denaturants, crowding agents, etc.) In embodiments, the reservoirs include sequencing reagents (such as an aqueous buffer containing enzymes, salts, and nucleotides); a wash solution (an aqueous buffer); a cleave solution (an aqueous buffer containing a cleaving agent, such as a reducing agent); or a cleaning solution (a dilute bleach solution, dilute NaOH solution, dilute HCl solution, dilute antibacterial solution, or water). The fluid of each of the reservoirs can vary. The fluid can be, for example, an aqueous solution which may contain buffers (e.g., saline-sodium citrate (SSC), ascorbic acid, tris(hydroxymethyl)aminomethane or “Tris”), aqueous salts (e.g., KCl or (NH₄)₂SO₄)), nucleotides, polymerases, cleaving agent (e.g., tri-n-butyl-phosphine, triphenyl phosphine and its sulfonated versions (i.e., tris(3-sulfophenyl)-phosphine, TPPTS), and tri(carboxyethyl)phosphine (TCEP) and its salts, cleaving agent scavenger compounds (e.g., 2′-Dithiobisethanamine or 11-Azido-3,6,9-trioxaundecane-1-amine), chelating agents (e.g., EDTA), detergents, surfactants, crowding agents, or stabilizers (e.g., PEG, Tween, BSA). Non-limited examples of reservoirs include cartridges, pouches, vials, containers, and eppendorf tubes. In embodiments, the device is configured to perform fluorescent imaging. In embodiments, the device includes one or more light sources (e.g., one or more lasers). In embodiments, the illuminator or light source is a radiation source (i.e., an origin or generator of propagated electromagnetic energy) providing incident light to the sample. A radiation source can include an illumination source producing electromagnetic radiation in the ultraviolet (UV) range (about 200 to 390 nm), visible (VIS) range (about 390 to 770 nm), or infrared (IR) range (about 0.77 to 25 microns), or other range of the electromagnetic spectrum. In embodiments, the illuminator or light source is a lamp such as an arc lamp or quartz halogen lamp. In embodiments, the illuminator or light source is a coherent light source. In embodiments, the light source is a laser, LED (light emitting diode), a mercury or tungsten lamp, or a super-continuous diode. In embodiments, the light source provides excitation beams having a wavelength between 200 nm to 1500 nm. In embodiments, the laser provides excitation beams having a wavelength of 405 nm, 470 nm, 488 nm, 514 nm, 520 nm, 532 nm, 561 nm, 633 nm, 639 nm, 640 nm, 800 nm, 808 nm, 912 nm, 1024 nm, or 1500 nm. In embodiments, the illuminator or light source is a light-emitting diode (LED). The LED can be, for example, an Organic Light Emitting Diode (OLED), a Thin Film Electroluminescent Device (TFELD), or a Quantum dot based inorganic organic LED. The LED can include a phosphorescent OLED (PHOLED). In embodiments, the nucleic acid sequencing device includes an imaging system (e.g., an imaging system as described herein). The imaging system capable of exciting one or more of the identifiable labels (e.g., a fluorescent label) linked to a nucleotide and thereafter obtain image data for the identifiable labels. The image data (e.g., detection data) may be analyzed by another component within the device. The imaging system may include a system described herein and may include a fluorescence spectrophotometer including an objective lens and/or a solid-state imaging device. The solid-state imaging device may include a charge coupled device (CCD) and/or a complementary metal oxide semiconductor (CMOS). The system may also include circuitry and processors, including systems using microcontrollers, reduced instruction set computers (RISC), application specific integrated circuits (ASICs), field programmable gate array (FPGAs), logic circuits, and any other circuit or processor capable of executing functions described herein. The set of instructions may be in the form of a software program. As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. In embodiments, the device includes a thermal control assembly useful to control the temperature of the reagents.

The term “image” is used according to its ordinary meaning and refers to a representation of all or part of an object. The representation may be an optically detected reproduction. For example, an image can be obtained from fluorescent, luminescent, scatter, or absorption signals. The part of the object that is present in an image can be the surface or other xy plane of the object. Typically, an image is a 2 dimensional representation of a 3 dimensional object. An image may include signals at differing intensities (i.e., signal levels). An image can be provided in a computer readable format or medium. An image is derived from the collection of focus points of light rays coming from an object (e.g., the sample), which may be detected by any image sensor.

As used herein, the term “signal” is intended to include, for example, fluorescent, luminescent, scatter, or absorption impulse or electromagnetic wave transmitted or received. Signals can be detected in the ultraviolet (UV) range (about 200 to 390 nm), visible (VIS) range (about 391 to 770 nm), infrared (IR) range (about 0.771 to 25 microns), or other range of the electromagnetic spectrum. The term “signal level” refers to an amount or quantity of detected energy or coded information. For example, a signal may be quantified by its intensity, wavelength, energy, frequency, power, luminance, or a combination thereof. Other signals can be quantified according to characteristics such as voltage, current, electric field strength, magnetic field strength, frequency, power, temperature, etc. Absence of signal is understood to be a signal level of zero or a signal level that is not meaningfully distinguished from noise.

The term “xy coordinates” refers to information that specifies location, size, shape, and/or orientation in an xy plane. The information can be, for example, numerical coordinates in a Cartesian system. The coordinates can be provided relative to one or both of the x and y axes or can be provided relative to another location in the xy plane (e.g., a fiducial). The term “xy plane” refers to a 2 dimensional area defined by straight line axes x and y. When used in reference to a detecting apparatus and an object observed by the detector, the xy plane may be specified as being orthogonal to the direction of observation between the detector and object being detected.

As used herein, the term “tissue section” refers to a piece of tissue that has been obtained from a subject, optionally fixed and attached to a surface, e.g., a microscope slide.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### II. Compositions & Kits

In an aspect, provided herein are kits for use in accordance with any of the compounds, compositions, or methods disclosed herein, and including one or more elements thereof. In embodiments, a kit includes labeled nucleotides including differently labeled nucleotides, enzymes, buffers, oligonucleotides, and related solvents and solutions. In embodiments, the kit includes one or more oligonucleotide probes (e.g., an oligonucleotide probe as described herein). The kit may also include a template nucleic acid (DNA and/or RNA), one or more primer polynucleotides, nucleoside triphosphates (including, e.g., deoxyribonucleotides, dideoxynucleotides, ribonucleotides, labeled nucleotides, and/or modified nucleotides), buffers, salts, and/or labels (e.g., fluorophores). In embodiments, the kit includes components useful for circularizing template polynucleotides using a ligation enzyme (e.g., Circligase enzyme, Taq DNA Ligase, HiFi Taq DNA Ligase, T4 ligase, SplintR ligase, or Ampligase DNA Ligase). For example, such a kit includes the following components: (a) reaction buffer for controlling pH and providing an optimized salt composition for a ligation enzyme (e.g., Circligase enzyme, Taq DNA Ligase, HiFi Taq DNA Ligase, T4 ligase, SplintR ligase, or Ampligase DNA Ligase), and (b) ligation enzyme cofactors. In embodiments, the kit further includes instructions for use thereof. In embodiments, kits described herein include a polymerase. In embodiments, the polymerase is a DNA polymerase. In embodiments, the DNA polymerase is a thermophilic nucleic acid polymerase. In embodiments, the DNA polymerase is a modified archacal DNA polymerase. In embodiments, the kit includes a sequencing solution. In embodiments, the sequencing solution include labeled nucleotides including differently labeled nucleotides, wherein the label (or lack thereof) identifies the type of nucleotide. For example, each adenine nucleotide, or analog thereof; a thymine nucleotide; a cytosine nucleotide, or analog thereof; and a guanine nucleotide, or analog thereof may be labeled with a different fluorescent label.

In an aspect is provided a kit including a first oligonucleotide, a second oligonucleotide, a first oligonucleotide probe, a second oligonucleotide probe, and a ligase, wherein the first oligonucleotide includes a first probe hybridization sequence, a barcode sequence, and a second probe hybridization sequence; the second oligonucleotide includes a third probe hybridization sequence and a fourth probe hybridization sequence; the first oligonucleotide probe includes a sequence complementary to the second probe hybridization sequence and a sequence complementary to the fourth probe hybridization sequence; and the second oligonucleotide probe includes a sequence complementary to the first probe hybridization sequence and a sequence complementary to the third probe hybridization sequence.

In an aspect is provided a kit including a first oligonucleotide, a second oligonucleotide, a first oligonucleotide probe, a second oligonucleotide probe, and a ligase, wherein the first oligonucleotide includes a first probe hybridization sequence 5′ to the first target hybridization sequence, a barcode sequence 5′ to the first probe hybridization sequence, and a second probe hybridization sequence 5′ to the barcode sequence; the second oligonucleotide includes a third probe hybridization sequence 3′ to the second target hybridization sequence and a fourth probe hybridization sequence 3′ to the third probe hybridization sequence; the first oligonucleotide probe includes a sequence complementary to the second probe hybridization sequence and a sequence complementary to the fourth probe hybridization sequence; and the second oligonucleotide probe includes a sequence complementary to the first probe hybridization sequence and a sequence complementary to the third probe hybridization sequence.

In an aspect is provided a kit including a first oligonucleotide, a second oligonucleotide, a first oligonucleotide probe, a second oligonucleotide probe, and a ligase, wherein the first oligonucleotide includes a first target hybridization sequence capable of hybridizing to a first sequence of a target polynucleotide, and a barcode sequence; the second oligonucleotide includes a second target hybridization sequence capable of hybridizing to a second sequence of the target polynucleotide; and the first oligonucleotide probe includes a sequence complementary to a 5′ end of the first oligonucleotide and a sequence complementary to a 3′ end of the second oligonucleotide; and the second oligonucleotide probe includes a sequence complementary to a sequence 5′ of the first target hybridization sequence and a sequence complementary to a sequence 3′ of the second target hybridization sequence.

In embodiments, the second oligonucleotide does not include a barcode sequence. In embodiments, the first oligonucleotide probe and second oligonucleotide probe do not include a barcode sequence. In embodiments, the second oligonucleotide includes a hybridization sequence (e.g., the third hybridization sequence) common to a plurality of second oligonucleotides.

In embodiments, the target hybridization sequence of each oligonucleotide is greater than 30 nucleotides. In embodiments, the target hybridization sequence of each oligonucleotide is about 5 to about 35 nucleotides in length. In embodiments, the target hybridization sequence is about 12 to 15 nucleotides in length. In embodiments, the target hybridization sequence is about 35 to 40 nucleotides in length to maximize specificity. In embodiments, the target hybridization sequence is greater than 12 nucleotides in length. In embodiments, the target hybridization sequence is about 5, about 10, about 15, about 20, about 25, about 30, or about 35 nucleotides in length. In embodiments, the target hybridization sequence of each oligonucleotide primer is a single stranded polynucleotide that is at least 50% complementary, at least 75% complementary, at least 85% complementary, at least 90% complementary, at least 95% complementary, at least 98%, at least 99% complementary, or 100% complementary to a portion of a target polynucleotide. In embodiments, the target hybridization sequence of each oligonucleotide (e.g., each oligonucleotide of a plurality of oligonucleotides) is complementary to different portions of the same target polynucleotide. In embodiments, the target hybridization sequence of each oligonucleotide (e.g., each oligonucleotide of a plurality of oligonucleotides) is complementary to different portions of different target polynucleotides. In embodiments, the target hybridization sequence of each oligonucleotide is complementary to portions of the same target polynucleotide that are separated by about 10 to about 500 nucleotides. In embodiments, the target hybridization sequence of each oligonucleotide are complementary to portions of the same target polynucleotide that are separated by about 10, about 15, about 20, about 25, about 30, about 35, about 40, about 45, about 50, about 75, about 100, about 125, about 150, about 175, about 200, about 225, about 250, about 275, about 300, about 325, about 350, about 375, about 400, about 425, about 450, about 475, or about 500 nucleotides. In embodiments, the target hybridization sequence of each oligonucleotide is complementary to portions of the same target polynucleotide that are separated by about or more than about 10, 15, 20, 25, 30, 35, 40, 45, 50, 75, 100, 125, 150, 175, 200, 225, 250, 275, 300, 325, 350, 375, 400, 425, 450, 475, or 500 nucleotides.

In embodiments, the first target sequence is 5′ to the second target sequence of the target polynucleotide. In embodiments, the first target sequence is about 10 to about 500 nucleotides 5′ to the second target sequence of the target polynucleotide. In embodiments, the first target sequence is about 10, about 15, about 20, about 25, about 30, about 35, about 40, about 45, about 50, about 75, about 100, about 125, about 150, about 175, about 200, about 225, about 250, about 275, about 300, about 325, about 350, about 375, about 400, about 425, about 450, about 475, or about 500 nucleotides 5′ to the second target sequence of the target polynucleotide. In embodiments, the first target sequence is about or more than about 10, 15, 20, 25, 30, 35, 40, 45, 50, 75, 100, 125, 150, 175, 200, 225, 250, 275, 300, 325, 350, 375, 400, 425, 450, 475, or 500 nucleotides 5′ to the second target sequence of the target polynucleotide.

In embodiments, the first target hybridization sequence of the first oligonucleotide and the second target hybridization sequence of the second oligonucleotide are each about 5 to about 35 nucleotides in length. In embodiments, the first oligonucleotide and the second oligonucleotide each include a target hybridization sequence (e.g., the first target hybridization sequence of the first oligonucleotide and the second target hybridization sequence of the second oligonucleotide) having 12 to 15 nucleotides in length. In embodiments, the first oligonucleotide and the second oligonucleotide each include a target hybridization sequence having 35 to 40 nucleotides in length to maximize specificity. In embodiments, the first oligonucleotide and the second oligonucleotide each include a target hybridization sequence greater than 12 nucleotides in length. In embodiments, the first oligonucleotide and the second oligonucleotide each include a target hybridization sequence having about 5, about 10, about 15, about 20, about 25, about 30, or about 35 nucleotides in length. In embodiments, the first oligonucleotide includes a target hybridization sequence (e.g., the first target hybridization sequence) having about 5, about 10, about 15, about 20, about 25, about 30, or about 35 nucleotides in length. In embodiments, the second oligonucleotide includes a target hybridization sequence (e.g., the second target hybridization sequence) having about 5, about 10, about 15, about 20, about 25, about 30, or about 35 nucleotides in length. In embodiments, the target hybridization sequence of the first oligonucleotide is a single stranded polynucleotide that is at least 50% complementary, at least 75% complementary, at least 85% complementary, at least 90% complementary, at least 95% complementary, at least 98%, at least 99% complementary, or 100% complementary to a portion of a target polynucleotide. In embodiments, the target hybridization sequence of the second oligonucleotide is a single stranded polynucleotide that is at least 50% complementary, at least 75% complementary, at least 85% complementary, at least 90% complementary, at least 95% complementary, at least 98%, at least 99% complementary, or 100% complementary to a portion of a target polynucleotide. In embodiments, the first target hybridization sequence and second target hybridization sequence are complementary to different portions of the same target polynucleotide. In embodiments, the first target hybridization sequence and second target hybridization sequence are complementary to different portions of different target polynucleotides. In embodiments, the first target hybridization sequence and second target hybridization sequence are complementary to portions of the same target polynucleotide that are separated by about 10 to about 500 nucleotides. In embodiments, the first target hybridization sequence and second target hybridization sequence are complementary to portions of the same target polynucleotide that are separated by about 10, about 15, about 20, about 25, about 30, about 35, about 40, about 45, about 50, about 75, about 100, about 125, about 150, about 175, about 200, about 225, about 250, about 275, about 300, about 325, about 350, about 375, about 400, about 425, about 450, about 475, or about 500 nucleotides. In embodiments, the first target hybridization sequence and second target hybridization sequence are complementary to portions of the same target polynucleotide that are separated by about or more than about 10, 15, 20, 25, 30, 35, 40, 45, 50, 75, 100, 125, 150, 175, 200, 225, 250, 275, 300, 325, 350, 375, 400, 425, 450, 475, or 500 nucleotides.

In embodiments, each oligonucleotide (e.g., each oligonucleotide of a plurality of oligonucleotide probes) includes a primer binding sequence. In embodiments, the first oligonucleotide and the second oligonucleotide each independently include a primer binding sequence. In embodiments, the first oligonucleotide and the second oligonucleotide each independently include a primer binding sequence, wherein the primer binding sequences are the same. In embodiments, the first oligonucleotide and the second oligonucleotide each independently include a primer binding sequence, wherein the primer binding sequences are different. In embodiments, the first oligonucleotide probe and the second oligonucleotide probe each independently include a primer binding sequence. In embodiments, the first oligonucleotide probe and the second oligonucleotide probe each independently include a primer binding sequence, wherein the primer binding sequences are the same. In embodiments, the first oligonucleotide probe and the second oligonucleotide probe each independently include a primer binding sequence, wherein the primer binding sequences are different.

In embodiments, each oligonucleotide includes about 50 to about 150 nucleotides. In embodiments, the first oligonucleotide and the second oligonucleotide each include about 50 to about 150 nucleotides. In embodiments, the first oligonucleotide probe and the second oligonucleotide probe each include about 50 to about 150 nucleotides. In embodiments, each oligonucleotide probe includes about 50 to about 300 nucleotides. In embodiments, the first oligonucleotide and the second oligonucleotide each include about 50 to about 300 nucleotides. In embodiments, the first oligonucleotide probe and the second oligonucleotide probe each include about 50 to about 300 nucleotides. In embodiments, the first oligonucleotide and the second oligonucleotide each include about 50 to about 300 nucleotides. In embodiments, the first oligonucleotide probe and the second oligonucleotide probe each include about 50 to about 300 nucleotides. In embodiments, each oligonucleotide probe includes about or more than about 50, 75, 100, 125, 150, 175, 200, 250, 300, 350, 400, or 500 nucleotides. In embodiments, the first oligonucleotide and the second oligonucleotide each include about or more than about 50, 75, 100, 125, 150, 175, 200, 250, 300, 350, 400, or 500 nucleotides. In embodiments, the first oligonucleotide probe and the second oligonucleotide probe each include about or more than about 50, 75, 100, 125, 150, 175, 200, 250, 300, 350, 400, or 500 nucleotides. In embodiments, each oligonucleotide probe includes less than about 50, 75, 100, 125, 150, 175, 200, 250, 300, 350, 400, or 500 nucleotides. In embodiments, the first oligonucleotide and the second oligonucleotide each include less than about 50, 75, 100, 125, 150, 175, 200, 250, 300, 350, 400, or 500 nucleotides. In embodiments, the first oligonucleotide probe and the second oligonucleotide probe each include less than about 50, 75, 100, 125, 150, 175, 200, 250, 300, 350, 400, or 500 nucleotides.

In embodiments, the first oligonucleotide and/or the second oligonucleotide includes a spacer sequence (e.g., an optional spacer sequence). In embodiments, the first oligonucleotide probe and/or the second oligonucleotide probe includes a spacer sequence (e.g., an optional spacer sequence). In embodiments, the spacer sequence is about 5 to about 75 nucleotides in length. In embodiments, the spacer sequence is about 10 to about 150 nucleotides in length. In embodiments, the spacer sequence is about 5, 10, 15, 20, 25, 30, 40, 50, 60, 75, 100, 125, or 150 nucleotides in length.

In embodiments, the first probe hybridization sequence of the first oligonucleotide is greater than 30 nucleotides. In embodiments, the first probe hybridization sequence of the first oligonucleotide is about 5 to about 35 nucleotides in length. In embodiments, the first probe hybridization sequence is about 12 to 15 nucleotides in length. In embodiments, the first probe hybridization sequence is about 35 to 40 nucleotides in length to maximize specificity. In embodiments, the first probe hybridization sequence is greater than 12 nucleotides in length. In embodiments, the first probe hybridization sequence is about 5, about 10, about 15, about 20, about 25, about 30, or about 35 nucleotides in length.

In embodiments, the second probe hybridization sequence of the first oligonucleotide is greater than 30 nucleotides. In embodiments, the second probe hybridization sequence of the first oligonucleotide is about 5 to about 35 nucleotides in length. In embodiments, the second probe hybridization sequence is about 12 to 15 nucleotides in length. In embodiments, the second probe hybridization sequence is about 35 to 40 nucleotides in length to maximize specificity. In embodiments, the second probe hybridization sequence is greater than 12 nucleotides in length. In embodiments, the second probe hybridization sequence is about 5, about 10, about 15, about 20, about 25, about 30, or about 35 nucleotides in length.

In embodiments, the third probe hybridization sequence of the second oligonucleotide is greater than 30 nucleotides. In embodiments, the third probe hybridization sequence of the second oligonucleotide is about 5 to about 35 nucleotides in length. In embodiments, the third probe hybridization sequence is about 12 to 15 nucleotides in length. In embodiments, the third probe hybridization sequence is about 35 to 40 nucleotides in length to maximize specificity. In embodiments, the third probe hybridization sequence is greater than 12 nucleotides in length. In embodiments, the third probe hybridization sequence is about 5, about 10, about 15, about 20, about 25, about 30, or about 35 nucleotides in length.

In embodiments, the fourth probe hybridization sequence of the second oligonucleotide is greater than 30 nucleotides. In embodiments, the fourth probe hybridization sequence of the second oligonucleotide is about 5 to about 35 nucleotides in length. In embodiments, the fourth probe hybridization sequence is about 12 to 15 nucleotides in length. In embodiments, the fourth probe hybridization sequence is about 35 to 40 nucleotides in length to maximize specificity. In embodiments, the fourth probe hybridization sequence is greater than 12 nucleotides in length. In embodiments, the fourth probe hybridization sequence is about 5, about 10, about 15, about 20, about 25, about 30, or about 35 nucleotides in length.

In embodiments, each oligonucleotide probe includes about 50 to about 150 nucleotides. In embodiments, each oligonucleotide probe includes about 50 to about 300 nucleotides. In embodiments, each oligonucleotide probe includes about 50 to about 500 nucleotides. In embodiments, each oligonucleotide probe includes about or more than about 50, 75, 100, 125, 150, 175, 200, 250, 300, 350, 400, or 500 nucleotides. In embodiments, each oligonucleotide probe includes less than about 50, 75, 100, 125, 150, 175, 200, 250, 300, 350, 400, or 500 nucleotides.

In embodiments, the first oligonucleotide includes about 50 to about 150 nucleotides. In embodiments, the first oligonucleotide includes about 50 to about 300 nucleotides. In embodiments, the first oligonucleotide includes about 50 to about 500 nucleotides. In embodiments, the first oligonucleotide includes about or more than about 50, 75, 100, 125, 150, 175, 200, 250, 300, 350, 400, or 500 nucleotides. In embodiments, the first oligonucleotide includes less than about 50, 75, 100, 125, 150, 175, 200, 250, 300, 350, 400, or 500 nucleotides.

In embodiments, the second oligonucleotide includes about 50 to about 150 nucleotides. In embodiments, the second oligonucleotide includes about 50 to about 300 nucleotides. In embodiments, the second oligonucleotide includes about 50 to about 500 nucleotides. In embodiments, the second oligonucleotide includes about or more than about 50, 75, 100, 125, 150, 175, 200, 250, 300, 350, 400, or 500 nucleotides. In embodiments, the second oligonucleotide includes less than about 50, 75, 100, 125, 150, 175, 200, 250, 300, 350, 400, or 500 nucleotides.

In embodiments, the first oligonucleotide probe includes about 50 to about 150 nucleotides. In embodiments, the first oligonucleotide probe includes about 50 to about 300 nucleotides. In embodiments, the first oligonucleotide probe includes about 50 to about 500 nucleotides. In embodiments, the first oligonucleotide probe includes about or more than about 50, 75, 100, 125, 150, 175, 200, 250, 300, 350, 400, or 500 nucleotides. In embodiments, the first oligonucleotide probe includes less than about 50, 75, 100, 125, 150, 175, 200, 250, 300, 350, 400, or 500 nucleotides.

In embodiments, the second oligonucleotide probe includes about 50 to about 150 nucleotides. In embodiments, the second oligonucleotide probe includes about 50 to about 300 nucleotides. In embodiments, the second oligonucleotide probe includes about 50 to about 500 nucleotides. In embodiments, the second oligonucleotide probe includes about or more than about 50, 75, 100, 125, 150, 175, 200, 250, 300, 350, 400, or 500 nucleotides. In embodiments, the second oligonucleotide probe includes less than about 50, 75, 100, 125, 150, 175, 200, 250, 300, 350, 400, or 500 nucleotides.

In embodiments, the first oligonucleotide probe and the second oligonucleotide probe each include at least one amplification primer binding sequence or at least one sequencing primer binding sequence. The amplification primer binding sequence refers to a nucleotide sequence that is complementary to a primer useful in initiating amplification (i.e., an amplification primer). Likewise, a sequencing primer binding sequence is a nucleotide sequence that is complementary to a primer useful in initiating sequencing (i.e., a sequencing primer). Primer binding sequences usually have a length in the range of between 3 to 36 nucleotides, also 5 to 24 nucleotides, also from 14 to 36 nucleotides. In embodiments, an amplification primer and a sequencing primer are complementary to the same primer binding sequence, or overlapping primer binding sequences. In embodiments, an amplification primer and a sequencing primer are complementary to different primer binding sequences.

In embodiments, each oligonucleotide (e.g., each oligonucleotide of a plurality of oligonucleotides targeting a sequence of a target polynucleotide) includes a barcode sequence. In embodiments, the first oligonucleotide includes a barcode sequence. In embodiments, the second oligonucleotide includes a barcode sequence. In embodiments, the first oligonucleotide probe includes a barcode sequence. In embodiments, the second oligonucleotide probe includes a barcode sequence.

In embodiments, the barcode (i.e., the barcode sequence) is at least 5, 6, 7, 8, 9, 10, 11, 12, 13, 14, or 15 nucleotides in length. In embodiments, the barcode is 5, 6, 7, 8, 9, 10, 11, 12, 13, 14, or 15 nucleotides in length. In embodiments, the barcode is 10 to 15 nucleotides in length. In embodiments, the barcode is at least about 1, 2, 3, 4, 5, 6, 7, 8, 9, 10, 15, 20, 25, 30, 35, 40, 45, 50 or more nucleotides in length. In embodiments, the barcode can be at most about 300, 200, 100, 90, 80, 70, 60, 50, 40, 30, 20, 15, 12, 10, 9, 8, 7, 6, 5, 4 or fewer or more nucleotides in length. In embodiments, the barcode includes between about 5 to about 8, about 5 to about 10, about 5 to about 15, about 5 to about 20, about 10 to about 150 nucleotides. In embodiments, the barcode includes between 5 to 8, 5 to 10, 5 to 15, 5 to 20, 10 to 150 nucleotides. In embodiments, the barcode is 10 nucleotides. In embodiments, the barcode may include a unique sequence (e.g., a barcode sequence) that gives the barcode its identifying functionality. The unique sequence may be random or non-random. Attachment of the barcode sequence (via binding of a proximity probe conjugated to the barcode sequence) to a protein or nucleic acid of interest (i.e., the target) may associate the barcode sequence with the protein or nucleic acid of interest. The barcode may then be used to identify the protein or nucleic acid of interest during sequencing, even when other proteins or nucleic acids of interest (e.g., including different oligonucleotide barcodes) are present. In embodiments, the barcode consists only of a unique barcode sequence. In embodiments, the 5′ end of a barcoded oligonucleotide is phosphorylated. In embodiments, the barcode is known (i.e., the nucleic sequence is known before sequencing) and is sorted into a basis-set according to their Hamming distance. Oligonucleotide barcodes (e.g., barcode sequences included in an oligonucleotide probe) can be associated with a target of interest by knowing, a priori, the target of interest, such as a gene or protein. In embodiments, the barcodes further include one or more sequences capable of specifically binding a gene or nucleic acid sequence of interest. For example, in embodiments, the barcode includes a sequence capable of hybridizing to mRNA, e.g., one containing a poly-T sequence (e.g., having several T's in a row, e.g., 4, 5, 6, 7, 8, or more T's).

In embodiments, the barcode is included as part of an oligonucleotide of longer sequence length, such as a primer or a random sequence (e.g., a random N-mer). In embodiments, the barcode contains random sequences to increase the mass or size of the oligonucleotide tag. The random sequence can be of any suitable length, and there may be one or more than one present. As non-limiting examples, the random sequence may have a length of 10 to 40, 10 to 30, 10 to 20, 25 to 50, 15 to 40, 15 to 30, 20 to 50, 20 to 40, or 20 to 30 nucleotides. In embodiments, each barcode sequence is selected from a known set of barcode sequences. In embodiments, each of the known set of barcode sequences is associated with a target hybridization sequence from a known set of target hybridization sequences. In embodiments, a first barcode sequence is associated with a first target hybridization sequence, and wherein a second barcode sequence is associated with a second target hybridization sequence (e.g., wherein the second target hybridization sequence is included in an oligonucleotide probe targeting a different target nucleic acid than the first target hybridization sequence). In embodiments, the same barcode sequence is associated with a plurality of oligonucleotide probes targeting different sequences of the same target nucleic acid (e.g., the same target polynucleotide).

In embodiments, the target nucleic acid (i.e., the target polynucleotide) includes a nucleic acid sequence encoding a TCR alpha chain, a TCR beta chain, a TCR delta chain, a TCR gamma chain, or any fragment thereof (e.g., variable regions including VDJ or VJ regions, constant regions, transmembrane regions, fragments thereof, combinations thereof, and combinations of fragments thereof). In embodiments, the target nucleic acid includes a nucleic acid sequence encoding a B cell receptor heavy chain, B cell receptor light chain, or any fragment thereof (e.g., variable regions including VDJ or VJ regions, constant regions, transmembrane regions, fragments thereof, combinations thereof, and combinations of fragments thereof). In embodiments, the target nucleic acid includes a CDR3 nucleic acid sequence. In embodiments, the target nucleic acid includes a TCRA gene sequence or a TCRB gene sequence. In embodiments, the target nucleic acid includes a TCRA gene sequence and a TCRB gene sequence. In embodiments, the target nucleic acid includes sequences of various T cell receptor alpha variable genes (TRAV genes), T cell receptor alpha joining genes (TRAJ genes), T cell receptor alpha constant genes (TRAC genes), T cell receptor beta variable genes (TRBV genes), T cell receptor beta diversity genes (TRBD genes), T cell receptor beta joining genes (TRBJ genes), T cell receptor beta constant genes (TRBC genes), T cell receptor gamma variable genes (TRGV genes), T cell receptor gamma joining genes (TRGJ genes), T cell receptor gamma constant genes (TRGC genes), T cell receptor delta variable genes (TRDV genes), T cell receptor delta diversity genes (TRDD genes), T cell receptor delta joining genes (TRDJ genes), or T cell receptor delta constant genes (TRDC genes).

In embodiments, the target polynucleotide includes a cancer-associated gene nucleic acid sequence, a viral nucleic acid sequence, a bacterial nucleic acid sequence, or a fungal nucleic acid sequence. In embodiments, the cancer-associated gene is a nucleic acid sequence identified within The Cancer Genome Atlas Program, accessible at www.cancer.gov/tcga.

In embodiments, the target polynucleotide includes a CD4, CD68, CD20, CD11c, CD8, HLA-DR, Ki67, CD45RO, PanCK, CD3e, CD44, CD45, HLA-A, CD14, CD56, CD57, CD19, CD2, CD1a, CD107a, CD21, Pax5, FOXP3, Granzyme B, CD38, CD39, CD79a, TIGIT, TOX, TP63, S100A4, TFAM, GP100, LaminB1, CK19, CK17, GATA3, SOX2, Bcl2, EpCAM, Caveolin, CD163, CD11b, MPO, CD141, iNOS, PD-1, PD-L1, ICOS, TIM3, LAG3, IDO1, CD40, HLA-E, IFNG, CD69, E-cadherin, CD31, Histone H3, Beta-actin, Podoplanin, SMA, Vimentin, Collagen IV, CD34, Beta-catenin, MMP-9, ZEB1, ASCT2, Na/K ATPase, HK1, LDHA, G6PD, IDH2, GLUT1, pNRF2, ATPA5, SDHA, Citrate Synthase, CPT1A, PARP, BAK, BCL-XL, BAX, BAD, Cytochrome c, LC3B, Beclin-1, H2AX, pRPS6, PCNA, Cyclin D1, HLA-DPB1, LEF1, GAL9, CD138, MC Tryptase, OX40, ZAP70, CD7, C₁Qa, CCR6, CD15, AXL, and/or CD227 nucleic acid sequence.

In embodiments, the target polynucleotide can include any polynucleotide of interest. The polynucleotide can include DNA, RNA, peptide nucleic acid, morpholino nucleic acid, locked nucleic acid, glycol nucleic acid, threose nucleic acid, mixtures thereof, and hybrids thereof. In embodiments, the polynucleotide is obtained from one or more source organisms. In some embodiments, the polynucleotide can include a selected sequence or a portion of a larger sequence. In embodiments, sequencing a portion of a polynucleotide or a fragment thereof can be used to identify the source of the polynucleotide. With reference to nucleic acids, polynucleotides and/or nucleotide sequences a “portion,” “fragment” or “region” can be at least 5 consecutive nucleotides, at least 10 consecutive nucleotides, at least 15 consecutive nucleotides, at least 20 consecutive nucleotides, at least 25 consecutive nucleotides, at least 50 consecutive nucleotides, at least 100 consecutive nucleotides, or at least 150 consecutive nucleotides.

In embodiments, the entire sequence of the target polynucleotide is about 1 to 3 kb, and only a portion of that target (e.g., 50 to 100 nucleotides) is sequenced. In embodiments, the target polynucleotide is about 1 to 3 kb. In embodiments, the target polynucleotide is about 1 to 2 kb. In embodiments, the target polynucleotide is about 1 kb. In embodiments, the target polynucleotide is about 2 kb. In embodiments, the target polynucleotide is less than 1 kb. In embodiments, the target polynucleotide is about 500 nucleotides. In embodiments, the target polynucleotide is about 200 nucleotides. In embodiments, the target polynucleotide is about 100 nucleotides. In embodiments, the target polynucleotide is less than 100 nucleotides. In embodiments, the target polynucleotide is about 5 to 50 nucleotides.

In embodiments, the target polynucleotide is an RNA nucleic acid sequence or DNA nucleic acid sequence. In embodiments, the target polynucleotide is an RNA nucleic acid sequence or DNA nucleic acid sequence from the same cell. In embodiments, the target polynucleotide is an RNA nucleic acid sequence. In embodiments, the RNA nucleic acid sequence is stabilized using known techniques in the art. For example, RNA degradation by RNase should be minimized using commercially available solutions, e.g., RNA Later®, RNA Lysis Buffer, or Keratinocyte serum-free medium). In embodiments, the target polynucleotide is messenger RNA (mRNA), transfer RNA (tRNA), micro RNA (miRNA), small interfering RNA (siRNA), small nucleolar RNA (snoRNA), small nuclear RNA (snRNA), Piwi-interacting RNA (piRNA), enhancer RNA (cRNA), or ribosomal RNA (rRNA). In embodiments, the target polynucleotide is pre-mRNA. In embodiments, the target polynucleotide is heterogeneous nuclear RNA (hnRNA). In embodiments, the target polynucleotide is mRNA, RNA (transfer RNA), rRNA (ribosomal RNA), or noncoding RNA (such as lncRNA (long noncoding RNA)). In embodiments, the target polynucleotides are on different regions of the same RNA nucleic acid sequence.

In embodiments, the target polynucleotide includes RNA nucleic acid sequences. In embodiments the target polynucleotide is an RNA transcript. In embodiments the target polynucleotide is a single stranded RNA nucleic acid sequence. In embodiments, the target polynucleotide is an RNA nucleic acid sequence or a DNA nucleic acid sequence (e.g., cDNA). In embodiments, the target polynucleotide is a cDNA target polynucleotide nucleic acid sequence and before step i), the RNA nucleic acid sequence is reverse transcribed to generate the cDNA target polynucleotide nucleic acid sequence. In embodiments, reverse transcription of the RNA nucleic acid is performed with a reverse transcriptase, for example, Tth DNA polymerase or mutants thereof. In embodiments, the target polynucleotide is genomic DNA (gDNA), mitochondrial DNA, chloroplast DNA, episomal DNA, viral DNA, or copy DNA (cDNA). In embodiments, the target polynucleotide is coding RNA such as messenger RNA (mRNA), and non-coding RNA (ncRNA) such as transfer RNA (tRNA), microRNA (miRNA), small nuclear RNA (snRNA), or ribosomal RNA (rRNA). In embodiments, the target polynucleotide is a cancer-associated gene. In embodiments, to minimize amplification errors or bias, the target polynucleotide is not reverse transcribed to generate cDNA.

In embodiments, each oligonucleotide (e.g., one or more oligonucleotide probes of a plurality of oligonucleotides) includes locked nucleic acids (LNAs), Bis-locked nucleic acids (bisLNAs), twisted intercalating nucleic acids (TINAs), bridged nucleic acids (BNAs), 2′-O-methyl RNA:DNA chimeric nucleic acids, minor groove binder (MGB) nucleic acids, morpholino nucleic acids, C5-modified pyrimidine nucleic acids, peptide nucleic acids (PNAs), or combinations thereof. In embodiments, the first oligonucleotide, the second oligonucleotide, the first oligonucleotide probe, and/or the second oligonucleotide probe includes locked nucleic acids (LNAs), Bis-locked nucleic acids (bisLNAs), twisted intercalating nucleic acids (TINAs), bridged nucleic acids (BNAs), 2′-O-methyl RNA:DNA chimeric nucleic acids, minor groove binder (MGB) nucleic acids, morpholino nucleic acids, C5-modified pyrimidine nucleic acids, peptide nucleic acids (PNAs), or combinations thereof.

In embodiments, each oligonucleotide probe includes one or more locked nucleic acid (LNA) nucleotides. In embodiments, the target hybridization sequence of each oligonucleotide probe includes one or more LNA nucleotides. In embodiments, the first probe hybridization sequence and/or the second probe hybridization sequence of each oligonucleotide probe includes one or more LNA nucleotides. In embodiments, the first target hybridization sequence of the first oligonucleotide and/or the second target hybridization sequence of the second oligonucleotide include one or more LNA nucleotides. In embodiments, the first oligonucleotide probe and/or the second oligonucleotide probe include one or more LNA nucleotides.

In embodiments, the target hybridization sequence and/or hybridization sequence includes Bis-locked nucleic acids (bisLNAs). In embodiments, the target hybridization sequence and/or hybridization sequence includes twisted intercalating nucleic acids (TINAs). In embodiments, the target hybridization sequence and/or hybridization sequence includes bridged nucleic acids (BNAs). In embodiments, the target hybridization sequence and/or hybridization sequence includes 2′-O-methyl RNA:DNA chimeric nucleic acids. In embodiments, the target hybridization sequence and/or hybridization sequence includes minor groove binder (MGB) nucleic acids. In embodiments, the target hybridization sequence and/or hybridization sequence includes morpholino nucleic acids. Morpholino nucleic acids are synthetic nucleotides that have standard nucleic acid bases (e.g., adenine, guanine, cytosine, and thymine) wherein those bases are bound to methylenemorpholine rings linked through phosphorodiamidate groups instead of phosphates. Morpholino nucleic acids may be referred to as phosphorodiamidate morpholino oligomers (PMOs). In embodiments, the target hybridization sequence and/or hybridization sequence includes C5-modified pyrimidine nucleic acids. In embodiments, the target hybridization sequence and/or hybridization sequence includes peptide nucleic acids (PNAs). In embodiments, the target hybridization sequence and/or hybridization sequence includes from 5′ to 3′ a plurality of synthetic nucleotides (e.g., LNAs) followed by a plurality (e.g., 2 to 5) canonical or native nucleotides (e.g., dNTPs). In embodiments, the target hybridization sequence and/or hybridization sequence includes one or more (e.g., 2 to 5) deoxyuracil nucleobases (dU). In embodiments, the one or more dU nucleobases are at or near the 3′ end of the target hybridization sequence and/or hybridization sequence (e.g., within 5 nucleotides of the 3′ end). In embodiments, the target hybridization sequence and/or hybridization sequence includes from 5′ to 3′ a plurality (e.g., 2 to 5) of phosphorothioate nucleic acids, followed by a plurality of synthetic nucleotides (e.g., LNAs), and subsequently followed by a plurality (e.g., 2 to 5) of canonical nucleobases. In some embodiments, the target hybridization sequence and/or hybridization sequence includes a plurality of canonical nucleobases, wherein the canonical nucleobases terminate (i.e., at the 3′ end) with a deoxyuracil nucleobase (dU).

In embodiments, the target hybridization sequence and/or hybridization sequence includes a plurality of LNAs interspersed throughout the polynucleotide. In embodiments, the target hybridization sequence and/or hybridization sequence includes a plurality of consecutive LNAs (e.g., 2 to 5 LNAs, 5 to 7 LNAs, or 7 to 10 LNAs) throughout the target hybridization sequence and/or hybridization sequence. In embodiments, the entire composition of the target hybridization sequence and/or hybridization sequence includes less than 70%, less than 60%, less than 50%, less than 40%, less than 30%, less than 20%, less than 10%, or less than 5% of LNAs. In embodiments, the entire composition of the target hybridization sequence and/or hybridization sequence includes up to about 70%, up to about 60%, up to about 50%, up to about 40%, up to about 30%, up to about 20%, up to about 10%, or up to about 5% of LNAs. In embodiments, the entire composition of the target hybridization sequence and/or hybridization sequence includes more than 60%, more than 50%, more than 40%, more than 30%, more than 20%, more than 10%, or more than 5% of LNAs. In embodiments, the entire composition of the target hybridization sequence and/or hybridization sequence includes about 5% to about 10%, about 10% to about 20%, about 20% to about 30%, about 30% to about 40%, about 40% to about 50%, about 50% to about 60%, or about 60% to about 70% of LNAs. In embodiments, the entire composition of the target hybridization sequence and/or hybridization sequence includes about 5%, about 10%, about 15%, about 20%, about 25%, about 30%, about 35%, about 40%, about 45%, about 50%, about 55%, about 60%, about 65%, or about 70% of LNAs. In embodiments, the entire composition of the target hybridization sequence and/or hybridization sequence includes about 30%, about 35%, about 40%, about 45%, about 50%, about 55%, about 60%, about 65%, about 70%, about 75%, about 80%, about 85%, about 90%, or about 95% of canonical dNTPs. In embodiments, the entire composition of the target hybridization sequence and/or hybridization sequence includes less than 95%, less than 90%, less than 80%, less than 70%, less than 60%, less than 50%, less than 40%, or less than 30% of canonical dNTPs. In embodiments, the entire composition of the target hybridization sequence and/or hybridization sequence includes up to about 95%, up to about 90%, up to about 80%, up to about 70%, up to about 60%, up to about 50%, up to about 40%, or up to about 30% of canonical dNTPs. In embodiments, the entire composition of the target hybridization sequence and/or hybridization sequence includes more than 90%, more than 80%, more than 70%, more than 60%, more than 50%, more than 40%, or more than 30% of canonical dNTPs.

In embodiments, the entire composition of the target hybridization sequence and/or hybridization sequence includes about 70% of LNAs and about 30% of canonical dNTPs. In embodiments, the entire composition of the target hybridization sequence and/or hybridization sequence includes about 65% of LNAs and about 35% of canonical dNTPs. In embodiments, the entire composition of the target hybridization sequence and/or hybridization sequence includes about 60% of LNAs and about 40% of canonical dNTPs. In embodiments, the entire composition of the target hybridization sequence and/or hybridization sequence includes about 55% of LNAs and about 45% of canonical dNTPs. In embodiments, the entire composition of the target hybridization sequence and/or hybridization sequence includes about 50% of LNAs and about 50% of canonical dNTPs. In embodiments, the entire composition of the target hybridization sequence and/or hybridization sequence includes about 45% of LNAs and about 55% of canonical dNTPs. In embodiments, the entire composition of the target hybridization sequence and/or hybridization sequence includes about 40% of LNAs and about 60% of canonical dNTPs. In embodiments, the entire composition of the target hybridization sequence and/or hybridization sequence includes about 35% of LNAs and about 65% of canonical dNTPs. In embodiments, the entire composition of the target hybridization sequence and/or hybridization sequence includes about 30% of LNAs and about 70% of canonical dNTPs. In embodiments, the entire composition of the target hybridization sequence and/or hybridization sequence includes about 25% of LNAs and about 75% of canonical dNTPs. In embodiments, the entire composition of the target hybridization sequence and/or hybridization sequence includes about 20% of LNAs and about 80% of canonical dNTPs. In embodiments, the entire composition of the target hybridization sequence and/or hybridization sequence includes about 15% of LNAs and about 85% of canonical dNTPs. In embodiments, the entire composition of the target hybridization sequence and/or hybridization sequence includes about 10% of LNAs and about 90% of canonical dNTPs. In embodiments, the entire composition of the target hybridization sequence and/or hybridization sequence includes about 5% of LNAs and about 95% of canonical dNTPs.

In embodiments, each oligonucleotide includes a blocking moiety at a 3′ end (e.g., at the 3′ end of each oligonucleotide of a plurality of oligonucleotides). In embodiments, the first oligonucleotide and/or the second oligonucleotide includes a blocking moiety at the 3′ end. In embodiments, the first oligonucleotide and the second oligonucleotide include a blocking moiety at the 3′ end. In embodiments, the first oligonucleotide probe includes a blocking moiety at the 3′ end. In embodiments, a terminal nucleotide of the first oligonucleotide includes a blocking moiety. In embodiments, a terminal nucleotide of the second oligonucleotide includes a blocking moiety. In embodiments, a terminal nucleotide of the first oligonucleotide probe includes a blocking moiety. In embodiments, the blocking moiety is reversible. In embodiments, the blocking moiety is irreversible. In embodiments, the blocking moiety at the 3′ end (e.g., the 3′ blocking moiety) includes a reversible terminator. In embodiments, the 3′ blocking moiety includes a dideoxynucleotide triphosphate (e.g., a ddNTP).

In embodiments, the kit includes a microplate, and reagents for sample preparation and purification, amplification, and/or sequencing (e.g., one or more sequencing reaction mixtures). In embodiments, the kit includes for protein detection includes a plurality of proximity probes linked to an oligonucleotide (e.g., DNA-conjugated antibodies).

In embodiments, amplification reagents and other reagents may be provided in lyophilized form. In embodiments, amplification reagents and other reagents may be provided in a container that includes wells within which the lyophilized reagent may be reconstituted.

In embodiments, the kit includes components useful for circularizing template polynucleotides using a ligation enzyme (e.g., Circligase enzyme, Taq DNA Ligase, HiFi Taq DNA Ligase, T4 ligase, SplintR ligase, or Ampligase DNA Ligase). For example, such a kit further includes the following components: (a) reaction buffer for controlling pH and providing an optimized salt composition for a ligation enzyme (e.g., Circligase enzyme, Taq DNA Ligase, HiFi Taq DNA Ligase, T4 ligase, SplintR ligase, or Ampligase DNA Ligase), and (b) ligation enzyme cofactors. In embodiments, the kit further includes instructions for use thereof. In embodiments, kits described herein include a polymerase. In embodiments, the polymerase is a DNA polymerase. In embodiments, the DNA polymerase is a thermophilic nucleic acid polymerase. In embodiments, the DNA polymerase is a modified archacal DNA polymerase. In embodiments, the kit includes a sequencing solution. In embodiments, the sequencing solution include labeled nucleotides including differently labeled nucleotides, wherein the label (or lack thereof) identifies the type of nucleotide. For example, each adenine nucleotide, or analog thereof; a thymine nucleotide; a cytosine nucleotide, or analog thereof; and a guanine nucleotide, or analog thereof may be labeled with a different fluorescent label. In embodiments, the kit includes a modified terminal deoxynucleotidyl transferase (TdT) enzyme.

In embodiments, the kit includes a sequencing polymerase, and one or more amplification polymerases. In embodiments, the sequencing polymerase is capable of incorporating modified nucleotides. In embodiments, the polymerase is a DNA polymerase. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol μ DNA polymerase, Pol λ DNA polymerase, Pol σ DNA polymerase, Pol α DNA polymerase, Pol δ DNA polymerase, Pol ε DNA polymerase, Pol η DNA polymerase, Pol ι DNA polymerase, Pol κ DNA polymerase, Pol ζ DNA polymerase, Pol γ DNA polymerase, Pol θ DNA polymerase, Pol ν DNA polymerase, or a thermophilic nucleic acid polymerase (e.g., Therminator γ, 9° N polymerase (exo-), Therminator II, Therminator III, or Therminator IX). In embodiments, the DNA polymerase is a thermophilic nucleic acid polymerase. In embodiments, the DNA polymerase is a modified archacal DNA polymerase. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044, each of which are incorporated herein by reference for all purposes). In embodiments, the kit includes a strand-displacing polymerase. In embodiments, the kit includes a strand-displacing polymerase, such as a phi29 polymerase, phi29 mutant polymerase or a thermostable phi29 mutant polymerase.

In embodiments, the kit includes a buffered solution. Typically, the buffered solutions contemplated herein are made from a weak acid and its conjugate base or a weak base and its conjugate acid. For example, sodium acetate and acetic acid are buffer agents that can be used to form an acetate buffer. Other examples of buffer agents that can be used to make buffered solutions include, but are not limited to, Tris, bicine, tricine, HEPES, TES, MOPS, MOPSO and PIPES. Additionally, other buffer agents that can be used in enzyme reactions, hybridization reactions, and detection reactions are known in the art. In embodiments, the buffered solution can include Tris. With respect to the embodiments described herein, the pH of the buffered solution can be modulated to permit any of the described reactions. In some embodiments, the buffered solution can have a pH greater than pH 7.0, greater than pH 7.5, greater than pH 8.0, greater than pH 8.5, greater than pH 9.0, greater than pH 9.5, greater than pH 10, greater than pH 10.5, greater than pH 11.0, or greater than pH 11.5. In other embodiments, the buffered solution can have a pH ranging, for example, from about pH 6 to about pH 9, from about pH 8 to about pH 10, or from about pH 7 to about pH 9. In embodiments, the buffered solution can include one or more divalent cations. Examples of divalent cations can include, but are not limited to, Mg2+, Mn2+, Zn2+, and Ca2+. In embodiments, the buffered solution can contain one or more divalent cations at a concentration sufficient to permit hybridization of a nucleic acid. In embodiments, the buffered solution can contain one or more divalent cations at a concentration sufficient to permit hybridization of a nucleic acid. In embodiments, the buffered solution includes about 10 mM Tris, about 20 mM Tris, about 30 mM Tris, about 40 mM Tris, or about 50 mM Tris. In embodiments the buffered solution includes about 50 mM NaCl, about 75 mM NaCl, about 100 mM NaCl, about 125 mM NaCl, about 150 mM NaCl, about 200 mM NaCl, about 300 mM NaCl, about 400 mM NaCl, or about 500 mM NaCl. In embodiments, the buffered solution includes about 0.05 mM EDTA, about 0.1 mM EDTA, about 0.25 mM EDTA, about 0.5 mM EDTA, about 1.0 mM EDTA, about 1.5 mM EDTA or about 2.0 mM EDTA. In embodiments, the buffered solution includes about 0.01% Triton X-100, about 0.025% Triton X-100, about 0.05% Triton X-100, about 0.1% Triton X-100, or about 0.5% Triton X-100. In embodiments, the buffered solution includes 20 mM Tris pH 8.0, 100 mM NaCl, 0.1 mM EDTA, 0.025% Triton X-100. In embodiments, the buffered solution includes 20 mM Tris pH 8.0, 150 mM NaCl, 0.1 mM EDTA, 0.025% Triton X-100. In embodiments, the buffered solution includes 20 mM Tris pH 8.0, 300 mM NaCl, 0.1 mM EDTA, 0.025% Triton X-100. In embodiments, the buffered solution includes 20 mM Tris pH 8.0, 400 mM NaCl, 0.1 mM EDTA, 0.025% Triton X-100. In embodiments, the buffered solution includes 20 mM Tris pH 8.0, 500 mM NaCl, 0.1 mM EDTA, 0.025% Triton X-100.

In embodiments, the kit includes one or more sequencing reaction mixtures. In embodiments, the sequencing reaction mixture includes a buffer. In embodiments, the buffer includes an acetate buffer, 3-(N-morpholino)propanesulfonic acid (MOPS) buffer, N-(2-Acetamido)-2-aminocthanesulfonic acid (ACES) buffer, phosphate-buffered saline (PBS) buffer, 4-(2-hydroxyethyl)-1-piperazineethanesulfonic acid (HEPES) buffer, N-(1,1-Dimethyl-2-hydroxyethyl)-3-amino-2-hydroxypropanesulfonic acid (AMPSO) buffer, borate buffer (e.g., borate buffered saline, sodium borate buffer, boric acid buffer), 2-Amino-2-methyl-1,3-propanediol (AMPD) buffer, N-cyclohexyl-2-hydroxyl-3-aminopropanesulfonic acid (CAPSO) buffer, 2-Amino-2-methyl-1-propanol (AMP) buffer, 4-(Cyclohexylamino)-1-butanesulfonic acid (CABS) buffer, glycine-NaOH buffer, N-Cyclohexyl-2-aminoethanesulfonic acid (CHES) buffer, tris(hydroxymethyl)aminomethane (Tris) buffer, or a N-cyclohexyl-3-aminopropanesulfonic acid (CAPS) buffer. In embodiments, the buffer is a borate buffer. In embodiments, the buffer is a CHES buffer. In embodiments, the sequencing reaction mixture includes nucleotides, wherein the nucleotides include a reversible terminating moiety and a label covalently linked to the nucleotide via a cleavable linker. In embodiments, the sequencing reaction mixture includes a buffer, DNA polymerase, detergent (e.g., Triton X), a chelator (e.g., EDTA), and/or salts (e.g., ammonium sulfate, magnesium chloride, sodium chloride, or potassium chloride).

In embodiments, the kit includes, without limitation, nucleic acid primers, probes, adapters, enzymes, and the like, and are each packaged in a container, such as, without limitation, a vial, tube or bottle, in a package suitable for commercial distribution, such as, without limitation, a box, a sealed pouch, a blister pack and a carton. The package typically contains a label or packaging insert indicating the uses of the packaged materials. As used herein, “packaging materials” includes any article used in the packaging for distribution of reagents in a kit, including without limitation containers, vials, tubes, bottles, pouches, blister packaging, labels, tags, instruction sheets and package inserts.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, digital storage medium, etc., on which the information has been recorded. Yet another means that may be present is a website address which may be used via the Internet to access the information at a removed site. Any convenient means may be present in the kits.

Adapters and/or primers may be supplied in the kits ready for use, as concentrates-requiring dilution before use, or in a lyophilized or dried form requiring reconstitution prior to use. If required, the kits may further include a supply of a suitable diluent for dilution or reconstitution of the primers and/or adapters. Optionally, the kits may further include supplies of reagents, buffers, enzymes, and dNTPs for use in carrying out nucleic acid amplification and/or sequencing. Further components which may optionally be supplied in the kit include sequencing primers suitable for sequencing templates prepared using the methods described herein.

In embodiments, the kit can further include one or more biological stain(s) (e.g., any of the biological stains as described herein). For example, the kit can further include eosin and hematoxylin. In other examples, the kit can include a biological stain such as acridine orange, Bismarck brown, carmine, coomassie blue, crystal violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, safranin, or any combination thereof.

In an aspect is provided a composition, the composition including: a first oligonucleotide hybridized to a first sequence of a target polynucleotide and a second oligonucleotide hybridized to a second sequence of the target polynucleotide, the first oligonucleotide including a first target hybridization sequence and a barcode sequence, wherein the first target hybridization sequence is complementary to the first sequence of the target polynucleotide, and the second oligonucleotide including a second target hybridization sequence, wherein the second target hybridization sequence is complementary to the second sequence of the target polynucleotide, wherein the first and second oligonucleotides are each hybridized to a first oligonucleotide probe and a second oligonucleotide probe, wherein the first oligonucleotide probe includes a sequence complementary to a 3′ end of the second oligonucleotide and a sequence complementary to a 5′ end of the first oligonucleotide, wherein the second oligonucleotide probe includes a sequence complementary to a sequence 5′ of the first target hybridization sequence and a sequence complementary to a sequence 3′ of the second target hybridization sequence, and wherein the barcode sequence is between the duplexed 5′ end of the first oligonucleotide probe and the duplexed 3′ end of the second oligonucleotide probe.

In embodiments, the composition is in a cell. In embodiments, the cell is attached to a substrate. In embodiments, the cell is attached to the substrate via a bioconjugate reactive moiety. In embodiments, the composition is within a cell or tissue sample. In embodiments, the cell or tissue sample is cleared (e.g., digested) of proteins, lipids, or proteins and lipids. In embodiments, the cell or tissue sample is processed according to a known technique in the art, for example CLARITY (Chung K., et al. Nature 497, 332-337 (2013)), PACT-PARS (Yang B et. al. Cell 158, 945-958 (2014).), CUBIC (Susaki E. A. et al. Cell 157, 726-739 (2014), 18), ScaleS (Hama H., et al. Nat. Neurosci. 18, 1518-1529 (2015)), OPTIClear (Lai H. M., et al. Nat. Commun. 9, 1066 (2018)), Ce3D (Li W., et al. Proc. Natl. Acad. Sci. U.S.A. 114, E7321-E7330 (2017)), BABB (Dodt H. U. et al. Nat. Methods 4, 331-336 (2007)), iDISCO (Renier N., et al. Cell 159, 896-910 (2014)), uDISCO (Pan C., et al. Nat. Methods 13, 859-867 (2016)), FluoClearBABB (Schwarz M. K., et al. PLOS ONE 10, c0124650 (2015)), Ethanol-ECi (Klingberg A., et al. J. Am. Soc. Nephrol. 28, 452-459 (2017)), and PEGASOS (Jing D. et al. Cell Res. 28, 803-818 (2018)).

### III. Methods

In an aspect is provided a method of profiling a sample (e.g., a cell). In embodiments, the method includes determining information (e.g., gene and protein expression) about the transcriptome of an organism thus elucidating subcellular substances and processes while gaining valuable spatial localization information within a cell. In embodiments, the method includes simultaneously sequencing a plurality of nucleic acids, such as RNA transcripts, in situ within an optically resolved volume of a sample (e.g., a voxel). RNA transcripts are responsible for the process of converting DNA into an organism's phenotype, thus by determining the types and quantity of RNA present in a sample (e.g., a cell), it is possible to assign a phenotype to the cell. RNA transcripts include coding RNA and non-coding RNA molecules, such as messenger RNA (mRNA), transfer RNA (tRNA), micro RNA (miRNA), small interfering RNA (siRNA), small nucleolar RNA (snoRNA), small nuclear RNA (snRNA), Piwi-interacting RNA (piRNA), enhancer RNA (eRNA), or ribosomal RNA (rRNA). In embodiments, the target is pre-mRNA. In embodiments, the target is heterogeneous nuclear RNA (hnRNA).

In an aspect is provided a method of detecting an RNA polynucleotide, the method including: a) hybridizing a first oligonucleotide to the target RNA polynucleotide, wherein the first oligonucleotide includes a first target hybridization sequence, a first probe hybridization sequence, a barcode sequence, and a second probe hybridization sequence; b) hybridizing a second oligonucleotide to the target RNA polynucleotide, wherein the second oligonucleotide includes a second target hybridization sequence, a third probe hybridization sequence, and a fourth probe hybridization sequence; c) hybridizing a first oligonucleotide probe to the first oligonucleotide and to the second oligonucleotide; d) hybridizing a second oligonucleotide probe to the first oligonucleotide and the second oligonucleotide; c) extending the second oligonucleotide probe along the barcode sequence with a polymerase to generate a complementary barcode sequence, ligating the complementary barcode sequence to the first oligonucleotide probe, and ligating the first oligonucleotide probe to the second oligonucleotide probe, thereby generating a circular oligonucleotide; f) amplifying the circular oligonucleotide, thereby generating an amplification product including multiple complements of the complementary barcode sequence; and g) sequencing the circular oligonucleotide, thereby detecting the RNA polynucleotide. In embodiments, each of the steps of the method are performed in situ. In embodiments, sequencing the circular oligonucleotide includes sequencing the barcode sequence. In embodiments, the barcode sequence is associated with the RNA polynucleotide.

In an aspect is provided a method of generating an amplification product, the method including: a) hybridizing a first oligonucleotide to a target polynucleotide, wherein the first oligonucleotide includes a first target hybridization sequence, a first probe hybridization sequence, a barcode sequence, and a second probe hybridization sequence; b) hybridizing a second oligonucleotide to the target polynucleotide, wherein the second oligonucleotide includes a second target hybridization sequence, a third probe hybridization sequence, and a fourth probe hybridization sequence; c) hybridizing a first oligonucleotide probe to the first oligonucleotide and to the second oligonucleotide; d) hybridizing a second oligonucleotide probe to the first oligonucleotide and the second oligonucleotide; c) extending the second oligonucleotide probe along the barcode sequence with a polymerase to generate a complementary barcode sequence, ligating the complementary barcode sequence to the first oligonucleotide probe, and ligating the first oligonucleotide probe to the second oligonucleotide probe, thereby generating a circular oligonucleotide; and f) amplifying the circular oligonucleotide, thereby generating an amplification product including multiple complements of the complementary barcode sequence. In embodiments, each of the steps of the method are performed in situ.

In an aspect is provided a method of generating an amplification product, the method including: a) contacting a target polynucleotide with a first oligonucleotide including a first target hybridization sequence, wherein the first oligonucleotide includes a first probe hybridization sequence, a barcode sequence, and a second probe hybridization sequence, and hybridizing the first target hybridization sequence to a first target sequence of the target polynucleotide; b) contacting the target polynucleotide with a second oligonucleotide including a second target hybridization sequence, wherein the second oligonucleotide includes a third probe hybridization sequence and a fourth probe hybridization sequence, and hybridizing the second target hybridization sequence to a second target sequence of the target polynucleotide; c) hybridizing a first oligonucleotide probe to the first oligonucleotide and to the second oligonucleotide, wherein the first oligonucleotide probe includes a sequence complementary to the second probe hybridization sequence and a sequence complementary to the fourth probe hybridization sequence; d) hybridizing a second oligonucleotide probe to the first oligonucleotide and the second oligonucleotide, wherein the second oligonucleotide probe includes a sequence complementary to the first probe hybridization sequence and a sequence complementary to the third probe hybridization sequence; c) extending the second oligonucleotide probe along the barcode sequence with a polymerase to generate a complementary barcode sequence, ligating the complementary barcode sequence to the first oligonucleotide probe, and ligating the first oligonucleotide probe to the second oligonucleotide probe, thereby generating a circular oligonucleotide; and f) amplifying the circular oligonucleotide, thereby generating an amplification product including multiple complements of the complementary barcode sequence. In embodiments, each of the steps of the method are performed in situ.

In an aspect is provided a method of generating an amplification product, the method including: a) contacting a target polynucleotide with a first oligonucleotide including a first target hybridization sequence, wherein the first oligonucleotide includes a first probe hybridization sequence 5′ to the first target hybridization sequence, a barcode sequence 5′ to the first probe hybridization sequence, and a second probe hybridization sequence 5′ to the barcode sequence, thereby hybridizing the first target hybridization sequence to a first target sequence of the target polynucleotide; b) contacting the target polynucleotide with a second oligonucleotide including a second target hybridization sequence, wherein the second oligonucleotide includes a third probe hybridization sequence 3′ to the second target hybridization sequence and a fourth probe hybridization sequence 3′ to the third probe hybridization sequence, thereby hybridizing the second probe hybridization sequence to a second target sequence of the target polynucleotide; c) hybridizing a first oligonucleotide probe to the first oligonucleotide and the second oligonucleotide, wherein the first oligonucleotide probe includes a sequence complementary to the second probe hybridization sequence and a sequence complementary to the fourth probe hybridization sequence; d) hybridizing a second oligonucleotide probe to the first oligonucleotide and the second oligonucleotide, wherein the second oligonucleotide probe includes a sequence complementary to the first probe hybridization sequence and a sequence complementary to the third probe hybridization sequence; c) extending the second oligonucleotide probe along the barcode sequence with a polymerase to generate a complementary barcode sequence, ligating the complementary barcode sequence to the first oligonucleotide probe, and ligating the first oligonucleotide probe to the second oligonucleotide probe, thereby generating a circular oligonucleotide; and f) amplifying the circular oligonucleotide, thereby generating an amplification product including multiple complements of the complementary barcode sequence. In embodiments, each of the steps of the method are performed in situ.

In an aspect is provided a method of amplifying a target polynucleotide sequence, the method including: a) contacting a target polynucleotide with a first oligonucleotide including a first target hybridization sequence and a second oligonucleotide including a second target hybridization sequence, wherein the first oligonucleotide includes a barcode sequence; b) hybridizing the first target hybridization sequence to a first sequence of the target polynucleotide, hybridizing the second probe hybridization sequence to a second sequence of the target polynucleotide, and hybridizing each of a first oligonucleotide probe and a second oligonucleotide probe to both the first oligonucleotide and the second oligonucleotide, wherein the first oligonucleotide probe includes a sequence complementary to a 3′ end of the second oligonucleotide and a sequence complementary to a 5′ end of the first oligonucleotide, wherein the second oligonucleotide probe includes a sequence complementary to a sequence 5′ of the first target hybridization sequence and a sequence complementary to a sequence 3′ of the second target hybridization sequence, and wherein the barcode sequence is between the duplexed 5′ end of the first oligonucleotide probe and the duplexed 3′ end of the second oligonucleotide probe; c) extending the second oligonucleotide probe along the barcode sequence with a polymerase to generate a complementary barcode sequence, ligating the complementary barcode sequence to the first oligonucleotide probe, and ligating the first oligonucleotide probe to the second oligonucleotide probe, thereby generating a circular oligonucleotide; and d) amplifying the circular oligonucleotide, thereby generating an amplification product including multiple complements of the complementary barcode sequence. In embodiments, each of the steps of the method are performed in situ.

In embodiments, the method further includes detecting the amplification product of step (f). In embodiments, the method further includes detecting the amplification product. In embodiments, detecting the amplification product includes hybridizing an oligonucleotide associated with a detectable label to the amplification product and identifying the detectable label (e.g., detecting the label, thereby detecting the amplification product). In embodiments, detecting includes serially contacting the amplification products with labeled probes (e.g., labeled oligonucleotides or labeled nucleotides).

In embodiments, the method further includes sequencing the amplification product of step (f). In embodiments, the method further includes sequencing the amplification product. In embodiments, sequencing includes sequencing by synthesis, sequencing by binding, sequencing by ligation, or pyrosequencing. In embodiments, sequencing includes extending a sequencing primer by incorporating a labeled nucleotide or labeled nucleotide analogue, and detecting the label to generate a signal for each incorporated nucleotide or nucleotide analogue, wherein the sequencing primer is hybridized to the extension product.

In embodiments, sequencing the amplification product includes sequencing the barcode sequence, or a complement thereof. In embodiments, sequencing the amplification product includes sequencing the first hybridization sequence, or a complement thereof.

In embodiments, steps (a)-(d) of the method are performed in or on a cell. In embodiments, steps (a)-(d) of the method are performed in or on a tissue.

In embodiments, the amplification product includes three or more copies of the target polynucleotide. In embodiments, the amplification product includes at least three or more copies of the target polynucleotide. In embodiments, the amplification product includes at least five or more copies of the target polynucleotide. In embodiments, the amplification product includes at 5 to 10 copies of the target polynucleotide. In embodiments, the amplification product includes 10 to 20 copies of the target polynucleotide. In embodiments, the amplification product includes 20 to 50 copies of the target polynucleotide.

In embodiments, extending the second oligonucleotide probe along the barcode sequence to generate a complementary barcode sequence includes extending the second oligonucleotide probe by using a polymerase. In embodiments, the polymerase is a DNA polymerase. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol μ DNA polymerase, Pol λ DNA polymerase, Pol σ DNA polymerase, Pol α DNA polymerase, Pol δ DNA polymerase, Pol ε DNA polymerase, Pol η DNA polymerase, Pol ι DNA polymerase, Pol κ DNA polymerase, Pol ζ DNA polymerase, Pol γ DNA polymerase, Pol θ DNA polymerase, Pol ν DNA polymerase, or a thermophilic nucleic acid polymerase (e.g., Therminator γ, 9° N polymerase (exo-), Therminator II, Therminator III, or Therminator IX). In embodiments, the DNA polymerase is a thermophilic nucleic acid polymerase. In embodiments, the polymerase is a bacterial DNA polymerase, eukaryotic DNA polymerase, archacal DNA polymerase, viral DNA polymerase, or phage DNA polymerases. Bacterial DNA polymerases include E. coli DNA polymerases I, II and III, IV and V, the Klenow fragment of E. coli DNA polymerase, Clostridium stercorarium (Cst) DNA polymerase, Clostridium thermocellum (Cth) DNA polymerase and Sulfolobus solfataricus (Sso) DNA polymerase. Eukaryotic DNA polymerases include DNA polymerases α, β, γ, δ, €, η, ζ, λ, σ, μ, and k, as well as the Revl polymerase (terminal deoxycytidyl transferase) and terminal deoxynucleotidyl transferase (TdT). Viral DNA polymerases include T4 DNA polymerase, phi-29 DNA polymerase, GA-1, phi-29-like DNA polymerases, PZA DNA polymerase, phi-15 DNA polymerase, Cp1 DNA polymerase, Cp1 DNA polymerase, T7 DNA polymerase, and T4 polymerase. Other useful DNA polymerases include thermostable and/or thermophilic DNA polymerases such as Thermus aquaticus (Taq) DNA polymerase, Thermus filiformis (Tfi) DNA polymerase, Thermococcus zilligi (Tzi) DNA polymerase, Thermus thermophilus (Tth) DNA polymerase, Thermus flavusu (Tfl) DNA polymerase, Pyrococcus woesci (Pwo) DNA polymerase, Pyrococcus furiosus (Pfu) DNA polymerase and Turbo Pfu DNA polymerase, Thermococcus litoralis (Tli) DNA polymerase, Pyrococcus sp. GB-D polymerase, Thermotoga maritima (Tma) DNA polymerase, Bacillus stearothermophilus (Bst) DNA polymerase, Pyrococcus Kodakaraensis (KOD) DNA polymerase, Pfx DNA polymerase, Thermococcus sp. JDF-3 (JDF-3) DNA polymerase, Thermococcus gorgonarius (Tgo) DNA polymerase, Thermococcus acidophilium DNA polymerase; Sulfolobus acidocaldarius DNA polymerase; Thermococcus sp. go N-7 DNA polymerase; Pyrodictium occultum DNA polymerase; Methanococcus voltae DNA polymerase; Methanococcus thermoautotrophicum DNA polymerase; Methanococcus jannaschii DNA polymerase; Desulfurococcus strain TOK DNA polymerase (D. Tok Pol); Pyrococcus abyssi DNA polymerase; Pyrococcus horikoshii DNA polymerase; Pyrococcus islandicum DNA polymerase; Thermococcus fumicolans DNA polymerase; Acropyrum pernix DNA polymerase; and the heterodimeric DNA polymerase DP1/DP2. In embodiments, the polymerase is 3PDX polymerase as disclosed in U.S. Pat. No. 8,703,461, the disclosure of which is incorporated herein by reference. In embodiments, the polymerase is a reverse transcriptase. Exemplary reverse transcriptases include, but are not limited to, HIV-1 reverse transcriptase from human immunodeficiency virus type 1 (PDB 1HMV), HIV-2 reverse transcriptase from human immunodeficiency virus type 2, M-MLV reverse transcriptase from the Moloney murine leukemia virus, AMV reverse transcriptase from the avian myeloblastosis virus, and Telomerase reverse transcriptase. In embodiments, the polymerase is a Thermus thermophilus (Tth) DNA polymerase or mutant thereof. In embodiments, the polymerase is a Reverse Transcription Xenopolymerase (RTX). In embodiments, the polymerase is a mutant M-MLV reverse transcriptase from the Moloney murine leukemia virus.

In embodiments, amplifying the circular oligonucleotide includes incubating the circular oligonucleotide with the strand-displacing polymerase (a) for about 1 minute to about 2 hours, and/or (b) at a temperature of about 20° ° C. to about 50° C. In embodiments, amplifying the circular oligonucleotide includes incubating the circular oligonucleotide with the strand-displacing polymerase for about 1 minute to about 2 hours. In embodiments, amplifying the circular oligonucleotide includes incubating the circular oligonucleotide with the strand-displacing polymerase for about 5, about 10, about 20, about 30, about 40, about 45, about 50, about 55, or about 60 minutes. In embodiments, amplifying the circular oligonucleotide includes incubating the circular oligonucleotide with the strand-displacing polymerase for about 5 minutes. In embodiments, amplifying the circular oligonucleotide includes incubating the circular oligonucleotide with the strand-displacing polymerase for about 10 minutes. In embodiments, amplifying the circular oligonucleotide includes incubating the circular oligonucleotide with the strand-displacing polymerase for about 20 minutes. In embodiments, amplifying the circular oligonucleotide includes incubating the circular oligonucleotide with the strand-displacing polymerase for about 30 minutes. In embodiments, amplifying the circular oligonucleotide includes incubating the circular oligonucleotide with the strand-displacing polymerase for about 45 minutes. In embodiments, amplifying the circular oligonucleotide includes incubating the circular oligonucleotide with the strand-displacing polymerase for about 60 minutes.

In embodiments, amplifying the circular oligonucleotide includes incubating the circular oligonucleotide with the strand-displacing polymerase for about 1 hour to about 12 hours. In embodiments, amplifying includes incubation with the strand-displacing polymerase for about 60 seconds to about 60 minutes. In embodiments, amplifying includes incubation with the strand-displacing polymerase for about 10 minutes to about 60 minutes. In embodiments, amplifying includes incubation with the strand-displacing polymerase for about 10 minutes to about 30 minutes. In embodiments, amplifying the circular oligonucleotide includes incubating the circular oligonucleotide with the strand-displacing polymerase for about 1, about 2, about 3, about 4, about 5, about 6, about 7, about 8, about 9, about 10, about 11, or about 12 hours. In embodiments, amplifying the circular oligonucleotide includes incubating the circular oligonucleotide with the strand-displacing polymerase for more than 12 hours.

In embodiments, amplifying the circular oligonucleotide includes incubating the circular oligonucleotide with the strand-displacing polymerase at a temperature of about 20° C. to about 50° C. In embodiments, incubation with the strand-displacing polymerase is at a temperature of about 20° C., about 25° C., about 30° C., about 35° C., about 40° C., about 45° C., or about 50° C. In embodiments, incubation with the strand-displacing polymerase is at a temperature of about 35° ° C. to 42° C. In embodiments, incubation with the strand-displacing polymerase is at a temperature of about 35° C., about 36° C., about 37° ° C., about 38° C., about 39ºC, about 40° ° C., about 41° C., or about 42° C. In embodiments, the strand-displacing polymerase is a phi29 polymerase, a SD polymerase, a Bst large fragment polymerase, phi29 mutant polymerase, a Thermus aquaticus polymerase, or a thermostable phi29 mutant polymerase.

In embodiments, amplifying includes rolling circle amplification (RCA) or rolling circle transcription (RCT) (see, e.g., Lizardi et al., Nat. Genet. 19:225-232 (1998), which is incorporated herein by reference in its entirety). Several suitable rolling circle amplification methods are known in the art. For example, RCA amplifies a circular polynucleotide (e.g., DNA) by polymerase extension of an amplification primer complementary to a portion of the template polynucleotide. This process generates copies of the circular polynucleotide template such that multiple complements of the template sequence arranged end to end in tandem are generated (i.e., a concatemer) locally preserved at the site of the circle formation. In embodiments, the amplifying occurs at isothermal conditions. In embodiments, the amplifying includes hybridization chain reaction (HCR). HCR uses a pair of complementary, kinetically trapped hairpin oligomers to propagate a chain reaction of hybridization events, as described in Dirks, R. M., & Pierce, N. A. (2004) PNAS USA, 101(43), 15275-15278, which is incorporated herein by reference for all purposes. In embodiments, the amplifying includes branched rolling circle amplification (BRCA); e.g., as described in Fan T, Mao Y, Sun Q, et al. Cancer Sci. 2018; 109:2897-2906, which is incorporated herein by reference in its entirety. In embodiments, the amplifying includes hyberbranched rolling circle amplification (HRCA). Hyperbranched RCA uses a second primer complementary to the first amplification product. This allows products to be replicated by a strand-displacement mechanism, which yields drastic amplification within an isothermal reaction (Lage et al., Genome Research 13:294-307 (2003), which is incorporated herein by reference in its entirety). In embodiments, amplifying includes polymerase extension of an amplification primer. In embodiments, the polymerase is T4, T7, Sequenase, Taq, Klenow, and Pol I DNA polymerases. SD polymerase, Bst large fragment polymerase, or a phi29 polymerase or mutant thereof. In embodiments, the strand-displacing enzyme is an SD polymerase, Bst large fragment polymerase, or a phi29 polymerase or mutant thereof. In embodiments, the strand-displacing polymerase is Bst DNA Polymerase Large Fragment, Thermus aquaticus (Taq) polymerase, or a mutant thereof. In embodiments, the strand-displacing polymerase is a phi29 polymerase, a phi29 mutant polymerase or a thermostable phi29 mutant polymerase. A “phi polymerase” (or “Φ29 polymerase”) is a DNA polymerase from the Φ29 phage or from one of the related phages that, like @29, contain a terminal protein used in the initiation of DNA replication. For example, phi29 polymerases include the B103, GA-1, PZA, Φ15, BS32, M2Y (also known as M2), Nf, G1, Cp-1, PRD1, PZE, SFS, Cp-5, Cp-7, PR4, PR5, PR722, L17, Φ21, and AV-1 DNA polymerases, as well as chimeras thereof. A phi29 mutant DNA polymerase includes one or more mutations relative to naturally-occurring wild-type phi29 DNA polymerases, for example, one or more mutations that alter interaction with and/or incorporation of nucleotide analogs, increase stability, increase read length, enhance accuracy, increase phototolerance, and/or alter another polymerase property, and can include additional alterations or modifications over the wild-type phi29 DNA polymerase, such as one or more deletions, insertions, and/or fusions of additional peptide or protein sequences. Thermostable phi29 mutant polymerases are known in the art, see for example US 2014/0322759, which is incorporated herein by reference for all purposes. For example, a thermostable phi29 mutant polymerase refers to an isolated bacteriophage phi29 DNA polymerase including at least one mutation selected from the group consisting of M8R, V51A, M97T, L123S, G197D, K209E, E221K, E239G, Q497P, K512E, E515A, and F526 (relative to wild type phi29 polymerase). In embodiments, the polymerase is a phage or bacterial RNA polymerases (RNAPs). In embodiments, the polymerase is a T7 RNA polymerase. In embodiments, the polymerase is an RNA polymerase. Useful RNA polymerases include, but are not limited to, viral RNA polymerases such as T7 RNA polymerase, T3 polymerase, SP6 polymerase, and Kll polymerase; Eukaryotic RNA polymerases such as RNA polymerase I, RNA polymerase II, RNA polymerase III, RNA polymerase IV, and RNA polymerase V; and Archaca RNA polymerase.

In embodiments, the amplification method includes a standard dNTP mixture including dATP, dCTP, dGTP and dTTP (for DNA) or dATP, dCTP, dGTP and dUTP (for RNA). In embodiments, the amplification method includes a mixture of standard dNTPs and modified nucleotides that contain functional moieties (e.g., bioconjugate reactive groups) that serve as attachment points to the cell or the matrix in which the cell is embedded (e.g. a hydrogel). In embodiments, the amplification method includes a mixture of standard dNTPs and modified nucleotides that contain functional moieties (e.g., bioconjugate reactive groups) that participate in the formation of a bioconjugate linker. The modified nucleotides may react and link the amplification product to the surrounding cell scaffold. For example, amplifying may include an extension reaction wherein the polymerase incorporates a modified nucleotide into the amplification product, wherein the modified nucleotide includes a bioconjugate reactive moiety (e.g., an alkynyl moiety) attached to the nucleobase. The bioconjugate reactive moiety of the modified nucleotide participates in the formation of a bioconjugate linker by reacting with a complementary bioconjugate reactive moiety present in the cell (e.g., a crosslinking agent, such as NHS-PEG-azide, or an amine moiety) thereby attaching the amplification product to the internal scaffold of the cell. In embodiments, the functional moiety can be covalently cross-linked, copolymerize with or otherwise non-covalently bound to the matrix. In embodiments, the functional moiety can react with a cross-linker. In embodiments, the functional moiety can be part of a ligand-ligand binding pair. Suitable exemplary functional moieties include an amine, acrydite, alkyne, biotin, azide, and thiol. In embodiments of crosslinking, the functional moiety is cross-linked to modified dNTP or dUTP or both. In embodiments, suitable exemplary cross-linker reactive groups include imidoester (DMP), succinimide ester (NHS), maleimide (Sulfo-SMCC), carbodiimide (DCC, EDC) and phenyl azide. Cross-linkers within the scope of the present disclosure may include a spacer moiety. In embodiments, such spacer moieties may be functionalized. In embodiments, such spacer moieties may be chemically stable. In embodiments, such spacer moieties may be of sufficient length to allow amplification of the nucleic acid bound to the matrix. In embodiments, suitable exemplary spacer moieties include polyethylene glycol, carbon spacers, photo-cleavable spacers and other spacers known to those of skill in the art and the like. In embodiments, amplification reactions include standard dNTPs and a modified nucleotide (e.g., amino-allyl dUTP, 5-TCO-PEG4-dUTP, C8-Alkyne-dUTP, 5-Azidomethyl-dUTP, 5-Vinyl-dUTP, or 5-Ethynyl dLTTP). For example, during amplification a mixture of standard dNTPs and aminoallyl deoxyuridine 5′-triphosphate (dUTP) nucleotides may be incorporated into the amplicon and subsequently cross-linked to the cell protein matrix by using a cross-linking reagent (e.g., an amine-reactive crosslinking agent with PEG spacers, such as (PEGylated bis(sulfosuccinimidyl)suberate) (BS(PEG)₉)).

In embodiments, the circular oligonucleotide is about 100 to about 1000 nucleotides in length. In embodiments, the circular oligonucleotide is about 100, about 200, about 300, about 400, about 500, about 600, about 700, about 800, about 900, or about 1000 nucleotides in length. In embodiments, the circular oligonucleotide is greater than 1000 nucleotides in length. In embodiments, the circular oligonucleotide is about or more than about 100, 150, 200, 250, 300, 350, 400, 500, 750, 1000, or more nucleotides in length. In embodiments, the circular oligonucleotide includes a plurality of sequencing primer binding sequences. In embodiments, the circular oligonucleotide includes a plurality of different sequencing primer binding sequences.

In embodiments, the target hybridization sequence of each oligonucleotide is about 5 to about 35 nucleotides in length. In embodiments, the target hybridization sequence is about 12 to 15 nucleotides in length. In embodiments, the target hybridization sequence is about 35 to 40 nucleotides in length to maximize specificity. In embodiments, the target hybridization sequence is greater than 12 nucleotides in length. In embodiments, the target hybridization sequence is about 5, about 10, about 15, about 20, about 25, about 30, or about 35 nucleotides in length. In embodiments, the target hybridization sequence of each oligonucleotide primer is a single stranded polynucleotide that is at least 50% complementary, at least 75% complementary, at least 85% complementary, at least 90% complementary, at least 95% complementary, at least 98%, at least 99% complementary, or 100% complementary to a portion of a target polynucleotide. In embodiments, the target hybridization sequence of each oligonucleotide (e.g., each oligonucleotide of a plurality of oligonucleotides) is complementary to different portions of the same target polynucleotide. In embodiments, the target hybridization sequence of each oligonucleotide (e.g., each oligonucleotide of a plurality of oligonucleotides) is complementary to different portions of different target polynucleotides. In embodiments, the target hybridization sequence of each oligonucleotide are complementary to portions of the same target polynucleotide that are separated by about 10 to about 500 nucleotides. In embodiments, the target hybridization sequence of each oligonucleotide are complementary to portions of the same target polynucleotide that are separated by about 10, about 15, about 20, about 25, about 30, about 35, about 40, about 45, about 50, about 75, about 100, about 125, about 150, about 175, about 200, about 225, about 250, about 275, about 300, about 325, about 350, about 375, about 400, about 425, about 450, about 475, or about 500 nucleotides. In embodiments, the target hybridization sequence of each oligonucleotide are complementary to portions of the same target polynucleotide that are separated by about or more than about 10, 15, 20, 25, 30, 35, 40, 45, 50, 75, 100, 125, 150, 175, 200, 225, 250, 275, 300, 325, 350, 375, 400, 425, 450, 475, or 500 nucleotides.

In embodiments, the first target hybridization sequence of the first oligonucleotide and the second target hybridization sequence of the second oligonucleotide are each about 5 to about 35 nucleotides in length. In embodiments, the first oligonucleotide and the second oligonucleotide each include a target hybridization sequence (e.g., the first target hybridization sequence of the first oligonucleotide and the second target hybridization sequence of the second oligonucleotide) having 12 to 15 nucleotides in length. In embodiments, the first oligonucleotide and the second oligonucleotide each include a target hybridization sequence having 35 to 40 nucleotides in length to maximize specificity. In embodiments, the first oligonucleotide and the second oligonucleotide each include a target hybridization sequence greater than 12 nucleotides in length. In embodiments, the first oligonucleotide and the second oligonucleotide each include a target hybridization sequence having about 5, about 10, about 15, about 20, about 25, about 30, or about 35 nucleotides in length. In embodiments, the first oligonucleotide includes a target hybridization sequence (e.g., the first target hybridization sequence) having about 5, about 10, about 15, about 20, about 25, about 30, or about 35 nucleotides in length. In embodiments, the second oligonucleotide includes a target hybridization sequence (e.g., the second target hybridization sequence) having about 5, about 10, about 15, about 20, about 25, about 30, or about 35 nucleotides in length. In embodiments, the target hybridization sequence of the first oligonucleotide is a single stranded polynucleotide that is at least 50% complementary, at least 75% complementary, at least 85% complementary, at least 90% complementary, at least 95% complementary, at least 98%, at least 99% complementary, or 100% complementary to a portion of a target polynucleotide. In embodiments, the target hybridization sequence of the second oligonucleotide is a single stranded polynucleotide that is at least 50% complementary, at least 75% complementary, at least 85% complementary, at least 90% complementary, at least 95% complementary, at least 98%, at least 99% complementary, or 100% complementary to a portion of a target polynucleotide. In embodiments, the first target hybridization sequence and second target hybridization sequence are complementary to different portions of the same target polynucleotide. In embodiments, the first target hybridization sequence and second target hybridization sequence are complementary to different portions of different target polynucleotides. In embodiments, the first target hybridization sequence and second target hybridization sequence are complementary to portions of the same target polynucleotide that are separated by about 10 to about 500 nucleotides. In embodiments, the first target hybridization sequence and second target hybridization sequence are complementary to portions of the same target polynucleotide that are separated by about 10, about 15, about 20, about 25, about 30, about 35, about 40, about 45, about 50, about 75, about 100, about 125, about 150, about 175, about 200, about 225, about 250, about 275, about 300, about 325, about 350, about 375, about 400, about 425, about 450, about 475, or about 500 nucleotides. In embodiments, the first target hybridization sequence and second target hybridization sequence are complementary to portions of the same target polynucleotide that are separated by about or more than about 10, 15, 20, 25, 30, 35, 40, 45, 50, 75, 100, 125, 150, 175, 200, 225, 250, 275, 300, 325, 350, 375, 400, 425, 450, 475, or 500 nucleotides.

In embodiments, each oligonucleotide (e.g., each oligonucleotide of a plurality of oligonucleotides) includes a primer binding sequence. In embodiments, the first oligonucleotide and the second oligonucleotide each independently include a primer binding sequence. In embodiments, the first oligonucleotide and the second oligonucleotide each independently include a primer binding sequence, wherein the primer binding sequences are the same. In embodiments, the first oligonucleotide and the second oligonucleotide each independently include a primer binding sequence, wherein the primer binding sequences are different. In embodiments, the first oligonucleotide and the second oligonucleotide probe each independently include a primer binding sequence. In embodiments, the first oligonucleotide probe and the second oligonucleotide probe each independently include a primer binding sequence, wherein the primer binding sequences are the same. In embodiments, the first oligonucleotide probe and the second oligonucleotide probe each independently include a primer binding sequence, wherein the primer binding sequences are different.

In embodiments, the first oligonucleotide probe and the second oligonucleotide probe each independently include at least one primer binding sequence. In embodiments, the first oligonucleotide probe and the second oligonucleotide probe each independently include at least two primer binding sequences. In embodiments, the first oligonucleotide probe and the second oligonucleotide probe each independently include a primer binding sequence from a known set of primer binding sequences. In embodiments, the first oligonucleotide probe and the second oligonucleotide probe each independently include at least two primer binding sequences from a known set of primer binding sequences. In embodiments, the first oligonucleotide probe and the second oligonucleotide probe each independently include up to 50 different primer binding sequences from a known set of primer binding sequences. In embodiments, the first oligonucleotide probe and the second oligonucleotide probe each independently include up to 10 different primer binding sequences from a known set of primer binding sequences. In embodiments, the first oligonucleotide probe and the second oligonucleotide probe each independently include up to 5 different primer binding sequences from a known set of primer binding sequences. In embodiments, the first oligonucleotide probe and the second oligonucleotide probe each independently include two or more sequencing primer binding sequences from a known set of sequencing primer binding sequences. In embodiments the first oligonucleotide probe and the second oligonucleotide probe each independently include 1, 2, 3, 4, 5, 6, 7, 8, 9, or 10 primer binding sequences from a known set of primer binding sequences. In embodiments, the first oligonucleotide probe and the second oligonucleotide probe each independently include two or more different primer binding sequences from a known set of primer binding sequences. In embodiments, the first oligonucleotide probe and the second oligonucleotide probe each independently include 1, 2, 3, 4, 5, 6, 7, 8, 9, or 10 different primer binding sequences from a known set of primer binding sequences. In embodiments, the first oligonucleotide probe and the second oligonucleotide probe each independently include 2 to 5 primer binding sequences from a known set of primer binding sequences. In embodiments, the first oligonucleotide probe and the second oligonucleotide probe each independently include 2 to 5 different primer binding sequences from a known set of primer binding sequences. In embodiments, the first oligonucleotide probe and the second oligonucleotide probe each independently include 2 to 5 sequencing primer binding sequences from a known set of sequencing primer binding sequences. In embodiments, the first oligonucleotide probe and the second oligonucleotide probe each independently include 2 to 5 different sequencing primer binding sequences from a known set of sequencing primer binding sequences. In embodiments, the first oligonucleotide probe and the second oligonucleotide probe each independently include at least two different primer binding sequences. In embodiments, the first oligonucleotide probe and the second oligonucleotide probe each independently include two different sequencing primer binding sequences.

In embodiments, the first oligonucleotide probe includes a primer binding sequence. In embodiments, the first oligonucleotide probe includes at least one primer binding sequence. In embodiments, the first oligonucleotide probe includes at least two primer binding sequences. In embodiments, the first oligonucleotide probe includes a primer binding sequence from a known set of primer binding sequences. In embodiments, the first oligonucleotide probe includes at least two primer binding sequences from a known set of primer binding sequences. In embodiments, the first oligonucleotide probe includes up to 50 different primer binding sequences from a known set of primer binding sequences. In embodiments, the first oligonucleotide probe includes up to 10 different primer binding sequences from a known set of primer binding sequences. In embodiments, the first oligonucleotide probe includes up to 5 different primer binding sequences from a known set of primer binding sequences. In embodiments, the first oligonucleotide probe includes two or more sequencing primer binding sequences from a known set of sequencing primer binding sequences. In embodiments, the first oligonucleotide probe includes 1, 2, 3, 4, 5, 6, 7, 8, 9, or 10 primer binding sequences from a known set of primer binding sequences. In embodiments, the first oligonucleotide probe includes two or more different primer binding sequences from a known set of primer binding sequences. In embodiments, the first oligonucleotide probe includes 1, 2, 3, 4, 5, 6, 7, 8, 9, or 10 different primer binding sequences from a known set of primer binding sequences. In embodiments, the first oligonucleotide probe includes 2 to 5 primer binding sequences from a known set of primer binding sequences. In embodiments, the first oligonucleotide probe includes 2 to 5 different primer binding sequences from a known set of primer binding sequences. In embodiments, the first oligonucleotide probe includes 2 to 5 sequencing primer binding sequences from a known set of sequencing primer binding sequences. In embodiments, the first oligonucleotide probe includes 2 to 5 different sequencing primer binding sequences from a known set of sequencing primer binding sequences. In embodiments, the first oligonucleotide probe includes at least two different primer binding sequences. In embodiments, the first oligonucleotide probe includes two different sequencing primer binding sequences.

In embodiments, the second oligonucleotide probe includes a primer binding sequence. In embodiments, the second oligonucleotide probe includes at least one primer binding sequence. In embodiments, the second oligonucleotide probe includes at least two primer binding sequences. In embodiments, the second oligonucleotide probe includes a primer binding sequence from a known set of primer binding sequences. In embodiments, the second oligonucleotide probe includes at least two primer binding sequences from a known set of primer binding sequences. In embodiments, the second oligonucleotide probe includes up to 50 different primer binding sequences from a known set of primer binding sequences. In embodiments, the second oligonucleotide probe includes up to 10 different primer binding sequences from a known set of primer binding sequences. In embodiments, the second oligonucleotide probe includes up to 5 different primer binding sequences from a known set of primer binding sequences. In embodiments, the second oligonucleotide probe includes two or more sequencing primer binding sequences from a known set of sequencing primer binding sequences. In embodiments, the second oligonucleotide probe includes 1, 2, 3, 4, 5, 6, 7, 8, 9, or 10 primer binding sequences from a known set of primer binding sequences. In embodiments, the second oligonucleotide probe includes two or more different primer binding sequences from a known set of primer binding sequences. In embodiments, the second oligonucleotide probe includes 1, 2, 3, 4, 5, 6, 7, 8, 9, or 10 different primer binding sequences from a known set of primer binding sequences. In embodiments, the second oligonucleotide probe includes 2 to 5 primer binding sequences from a known set of primer binding sequences. In embodiments, the second oligonucleotide probe includes 2 to 5 different primer binding sequences from a known set of primer binding sequences. In embodiments, the second oligonucleotide probe includes 2 to 5 sequencing primer binding sequences from a known set of sequencing primer binding sequences. In embodiments, the second oligonucleotide probe includes 2 to 5 different sequencing primer binding sequences from a known set of sequencing primer binding sequences. In embodiments, the second oligonucleotide probe includes at least two different primer binding sequences. In embodiments, the second oligonucleotide probe includes two different sequencing primer binding sequences.

In embodiments, each oligonucleotide probe includes about 50 to about 150 nucleotides. In embodiments, the first oligonucleotide and the second oligonucleotide each include about 50 to about 150 nucleotides. In embodiments, the first oligonucleotide probe and the second oligonucleotide probe each include about 50 to about 150 nucleotides. In embodiments, each oligonucleotide probe includes about 50 to about 300 nucleotides. In embodiments, the first oligonucleotide and the second oligonucleotide each include about 50 to about 300 nucleotides. In embodiments, the first oligonucleotide probe and the second oligonucleotide probe each include about 50 to about 300 nucleotides. In embodiments, the first oligonucleotide and the second oligonucleotide each include about 50 to about 300 nucleotides. In embodiments, the first oligonucleotide probe and the second oligonucleotide probe each include about 50 to about 300 nucleotides. In embodiments, each oligonucleotide probe includes about or more than about 50, 75, 100, 125, 150, 175, 200, 250, 300, 350, 400, or 500 nucleotides. In embodiments, the first oligonucleotide and the second oligonucleotide each include about or more than about 50, 75, 100, 125, 150, 175, 200, 250, 300, 350, 400, or 500 nucleotides. In embodiments, the first oligonucleotide probe and the second oligonucleotide probe each include about or more than about 50, 75, 100, 125, 150, 175, 200, 250, 300, 350, 400, or 500 nucleotides. In embodiments, each oligonucleotide probe includes less than about 50, 75, 100, 125, 150, 175, 200, 250, 300, 350, 400, or 500 nucleotides. In embodiments, the first oligonucleotide and the second oligonucleotide each include less than about 50, 75, 100, 125, 150, 175, 200, 250, 300, 350, 400, or 500 nucleotides. In embodiments, the first oligonucleotide probe and the second oligonucleotide probe each include less than about 50, 75, 100, 125, 150, 175, 200, 250, 300, 350, 400, or 500 nucleotides.

In embodiments, the first oligonucleotide and/or the second oligonucleotide includes a spacer sequence (e.g., an optional spacer sequence). In embodiments, the first oligonucleotide probe and/or the second oligonucleotide probe includes a spacer sequence (e.g., an optional spacer sequence). In embodiments, the spacer sequence is about 5 to about 75 nucleotides in length. In embodiments, the spacer sequence is about 10 to about 150 nucleotides in length. In embodiments, the spacer sequence is about 5, 10, 15, 20, 25, 30, 40, 50, 60, 75, 100, 125, or 150 nucleotides in length.

In embodiments, the first probe hybridization sequence of the first oligonucleotide is greater than 30 nucleotides. In embodiments, the first probe hybridization sequence of the first oligonucleotide is about 5 to about 35 nucleotides in length. In embodiments, the first probe hybridization sequence is about 12 to 15 nucleotides in length. In embodiments, the first probe hybridization sequence is about 35 to 40 nucleotides in length to maximize specificity. In embodiments, the first probe hybridization sequence is greater than 12 nucleotides in length. In embodiments, the first probe hybridization sequence is about 5, about 10, about 15, about 20, about 25, about 30, or about 35 nucleotides in length.

In embodiments, the second probe hybridization sequence of the first oligonucleotide is greater than 30 nucleotides. In embodiments, the second probe hybridization sequence of the first oligonucleotide is about 5 to about 35 nucleotides in length. In embodiments, the second probe hybridization sequence is about 12 to 15 nucleotides in length. In embodiments, the second probe hybridization sequence is about 35 to 40 nucleotides in length to maximize specificity. In embodiments, the second probe hybridization sequence is greater than 12 nucleotides in length. In embodiments, the second probe hybridization sequence is about 5, about 10, about 15, about 20, about 25, about 30, or about 35 nucleotides in length.

In embodiments, the third probe hybridization sequence of the second oligonucleotide is greater than 30 nucleotides. In embodiments, the third probe hybridization sequence of the second oligonucleotide is about 5 to about 35 nucleotides in length. In embodiments, the third probe hybridization sequence is about 12 to 15 nucleotides in length. In embodiments, the third probe hybridization sequence is about 35 to 40 nucleotides in length to maximize specificity. In embodiments, the third probe hybridization sequence is greater than 12 nucleotides in length. In embodiments, the third probe hybridization sequence is about 5, about 10, about 15, about 20, about 25, about 30, or about 35 nucleotides in length.

In embodiments, the fourth probe hybridization sequence of the second oligonucleotide is greater than 30 nucleotides. In embodiments, the fourth probe hybridization sequence of the second oligonucleotide is about 5 to about 35 nucleotides in length. In embodiments, the fourth probe hybridization sequence is about 12 to 15 nucleotides in length. In embodiments, the fourth probe hybridization sequence is about 35 to 40 nucleotides in length to maximize specificity. In embodiments, the fourth probe hybridization sequence is greater than 12 nucleotides in length. In embodiments, the fourth probe hybridization sequence is about 5, about 10, about 15, about 20, about 25, about 30, or about 35 nucleotides in length.

In embodiments, each oligonucleotide probe includes about 50 to about 150 nucleotides. In embodiments, each oligonucleotide probe includes about 50 to about 300 nucleotides. In embodiments, each oligonucleotide probe includes about 50 to about 500 nucleotides. In embodiments, each oligonucleotide probe includes about or more than about 50, 75, 100, 125, 150, 175, 200, 250, 300, 350, 400, or 500 nucleotides. In embodiments, each oligonucleotide probe includes less than about 50, 75, 100, 125, 150, 175, 200, 250, 300, 350, 400, or 500 nucleotides.

In embodiments, the first oligonucleotide includes about 50 to about 150 nucleotides. In embodiments, the first oligonucleotide includes about 50 to about 300 nucleotides. In embodiments, the first oligonucleotide includes about 50 to about 500 nucleotides. In embodiments, the first oligonucleotide includes about or more than about 50, 75, 100, 125, 150, 175, 200, 250, 300, 350, 400, or 500 nucleotides. In embodiments, the first oligonucleotide includes less than about 50, 75, 100, 125, 150, 175, 200, 250, 300, 350, 400, or 500 nucleotides.

In embodiments, the second oligonucleotide includes about 50 to about 150 nucleotides. In embodiments, the second oligonucleotide includes about 50 to about 300 nucleotides. In embodiments, the second oligonucleotide includes about 50 to about 500 nucleotides. In embodiments, the second oligonucleotide includes about or more than about 50, 75, 100, 125, 150, 175, 200, 250, 300, 350, 400, or 500 nucleotides. In embodiments, the second oligonucleotide includes less than about 50, 75, 100, 125, 150, 175, 200, 250, 300, 350, 400, or 500 nucleotides.

In embodiments, the first oligonucleotide probe includes about 50 to about 150 nucleotides. In embodiments, the first oligonucleotide probe includes about 50 to about 300 nucleotides. In embodiments, the first oligonucleotide probe includes about 50 to about 500 nucleotides. In embodiments, the first oligonucleotide probe includes about or more than about 50, 75, 100, 125, 150, 175, 200, 250, 300, 350, 400, or 500 nucleotides. In embodiments, the first oligonucleotide probe includes less than about 50, 75, 100, 125, 150, 175, 200, 250, 300, 350, 400, or 500 nucleotides.

In embodiments, the second oligonucleotide probe includes about 50 to about 150 nucleotides. In embodiments, the second oligonucleotide probe includes about 50 to about 300 nucleotides. In embodiments, the second oligonucleotide probe includes about 50 to about 500 nucleotides. In embodiments, the second oligonucleotide probe includes about or more than about 50, 75, 100, 125, 150, 175, 200, 250, 300, 350, 400, or 500 nucleotides. In embodiments, the second oligonucleotide probe includes less than about 50, 75, 100, 125, 150, 175, 200, 250, 300, 350, 400, or 500 nucleotides.

In embodiments, the first oligonucleotide probe and the second oligonucleotide probe each include at least one amplification primer binding sequence or at least one sequencing primer binding sequence. The amplification primer binding sequence refers to a nucleotide sequence that is complementary to a primer useful in initiating amplification (i.e., an amplification primer). Likewise, a sequencing primer binding sequence is a nucleotide sequence that is complementary to a primer useful in initiating sequencing (i.e., a sequencing primer). Primer binding sequences usually have a length in the range of between 3 to 36 nucleotides, also 5 to 24 nucleotides, also from 14 to 36 nucleotides. In embodiments, an amplification primer and a sequencing primer are complementary to the same primer binding sequence, or overlapping primer binding sequences. In embodiments, an amplification primer and a sequencing primer are complementary to different primer binding sequences.

In embodiments, each oligonucleotide (e.g., each oligonucleotide of a plurality of oligonucleotides targeting a sequence of a target polynucleotide) includes a barcode sequence. In embodiments, the first oligonucleotide includes a barcode sequence. In embodiments, the second oligonucleotide includes a barcode sequence. In embodiments, the first oligonucleotide probe includes a barcode sequence. In embodiments, the second oligonucleotide probe includes a barcode sequence. In embodiments, the barcode sequence is a nucleic acid sequence (e.g., 8 to 24 nucleotides) from a known set of barcode sequences. In embodiments, each barcode sequence is unique within the known set of barcodes. In embodiments, the barcodes are selected to form a known set of barcodes, e.g., the set of barcodes may be distinguished by a particular Hamming distance.

In embodiments, the barcode (i.e., the barcode sequence) is at least 5, 6, 7, 8, 9, 10, 11, 12, 13, 14, or 15 nucleotides in length. In embodiments, the barcode is 5, 6, 7, 8, 9, 10, 11, 12, 13, 14, or 15 nucleotides in length. In embodiments, the barcode is 10 to 15 nucleotides in length. In embodiments, the barcode is at least about 1, 2, 3, 4, 5, 6, 7, 8, 9, 10, 15, 20, 25, 30, 35, 40, 45, 50 or more nucleotides in length. In embodiments, the barcode can be at most about 300, 200, 100, 90, 80, 70, 60, 50, 40, 30, 20, 15, 12, 10, 9, 8, 7, 6, 5, 4 or fewer or more nucleotides in length. In embodiments, the barcode includes between about 5 to about 8, about 5 to about 10, about 5 to about 15, about 5 to about 20, about 10 to about 150 nucleotides. In embodiments, the barcode includes between 5 to 8, 5 to 10, 5 to 15, 5 to 20, 10 to 150 nucleotides. In embodiments, the barcode is 10 nucleotides. In embodiments, the barcode may include a unique sequence (e.g., a barcode sequence) that gives the barcode its identifying functionality. The unique sequence may be random or non-random. Attachment of the barcode sequence (via binding of a proximity probe conjugated to the barcode sequence) to a protein or nucleic acid of interest (i.e., the target) may associate the barcode sequence with the protein or nucleic acid of interest. The barcode may then be used to identify the protein or nucleic acid of interest during sequencing, even when other proteins or nucleic acids of interest (e.g., including different oligonucleotide barcodes) are present. In embodiments, the barcode consists only of a unique barcode sequence. In embodiments, the 5′ end of a barcoded oligonucleotide is phosphorylated. In embodiments, the barcode is known (i.e., the nucleic sequence is known before sequencing) and is sorted into a basis-set according to their Hamming distance. Oligonucleotide barcodes (e.g., barcode sequences included in an oligonucleotide probe) can be associated with a target of interest by knowing, a priori, the target of interest, such as a gene or protein. In embodiments, the barcodes further include one or more sequences capable of specifically binding a gene or nucleic acid sequence of interest. For example, in embodiments, the barcode includes a sequence capable of hybridizing to mRNA, e.g., one containing a poly-T sequence (e.g., having several T's in a row, e.g., 4, 5, 6, 7, 8, or more T's).

In embodiments, the barcode is included as part of an oligonucleotide of longer sequence length, such as a primer or a random sequence (e.g., a random N-mer). In embodiments, the barcode contains random sequences to increase the mass or size of the oligonucleotide tag. The random sequence can be of any suitable length, and there may be one or more than one present. As non-limiting examples, the random sequence may have a length of 10 to 40, 10 to 30, 10 to 20, 25 to 50, 15 to 40, 15 to 30, 20 to 50, 20 to 40, or 20 to 30 nucleotides. In embodiments, each barcode sequence is selected from a known set of barcode sequences. In embodiments, each of the known set of barcode sequences is associated with a target hybridization sequence from a known set of target hybridization sequences. In embodiments, a first barcode sequence is associated with a first target hybridization sequence, and wherein a second barcode sequence is associated with a second target hybridization sequence (e.g., wherein the second target hybridization sequence is included in an oligonucleotide probe targeting a different target nucleic acid than the first target hybridization sequence). In embodiments, the same barcode sequence is associated with a plurality of oligonucleotide probes targeting different sequences of the same target nucleic acid (e.g., the same target polynucleotide).

In embodiments, the barcode is taken from a “pool” or “set” or “basis-set” of potential oligonucleotide barcode sequences. The set of barcodes may be selected using any suitable technique, e.g., randomly, or such that the sequences allow for error detection and/or correction, or having a particular feature, such as by being separated by a certain distance (e.g., Hamming distance). In embodiments, the method includes selecting a basis-set of oligonucleotide barcodes having a specified Hamming distance (e.g., a Hamming distance of 10; a Hamming distance of 5). The pool may have any number of potential barcode sequences, e.g., at least 100, at least 300, at least 500, at least 1,000, at least 3,000, at least 5,000, at least 10,000, at least 30,000, at least 50,000, at least 100,000, at least 300,000, at least 500,000, or at least 1,000,000 barcode sequences. In embodiments, a barcode is a degenerate or partially-degenerate sequence, such that one or more nucleotides are selected at random from a set of two or more different nucleotides at one or more positions, with each of the different nucleotides selected at one or more positions represented in a pool of oligonucleotides including the degenerate or partially-degenerate sequence. The number of possible barcodes in a given set of barcodes will vary with the number of degenerate positions, and the number of bases permitted at each such position. For example, a barcode of five nucleotides (consecutive or non-consecutive), in which each position can be any of A, T, G, or C represents 5⁴, or 1024 possible barcodes. In embodiments, certain barcode sequences may be excluded from a pool, such as barcodes in which every position is the same base. In embodiments, there are about, 10², 10³ 10⁴, 10⁵, 10⁶, 10⁷, 10⁸, 10⁹, or a number or a range between any two of these values, unique nucleotide barcode sequences. In embodiments, there are at least, or at most 10², 10³ 10⁴, 10⁵, 10⁶, 10⁷, 10⁸, 10⁹ unique barcode sequences. In embodiments, a barcode is about, 1, 2, 3, 4, 5, 10, 15, 20, 25, 30, 35, 40, 45, 50, or a number or a range between any two of these values, nucleotides in length. A barcode can be at least, or at most, 1, 2, 3, 4, 5, 10, 15, 20, 25, 30, 35, 40, 45, 50, 100, or 200 nucleotides in length.

In embodiments, the barcodes in the known set of barcodes have a specified Hamming distance. In embodiments, the Hamming distance is 4 to 15. In embodiments, the Hamming distance is 8 to 12. In embodiments, the Hamming distance is 10. In embodiments, the Hamming distance is 0 to 100. In embodiments, the Hamming distance is 0 to 15. In embodiments, the Hamming distance is 0 to 10. In embodiments, the Hamming distance is 1 to 10. In embodiments, the Hamming distance is 5 to 10. In embodiments, the Hamming distance is 1 to 100. In embodiments, the Hamming distance between any two barcode sequences of the set is at least 2, 3, 4, or 5. In embodiments, the Hamming distance between any two barcode sequences of the set is at least 3. In embodiments, the Hamming distance between any two barcode sequences of the set is at least 4.

In embodiments, the target nucleic acid (i.e., the target polynucleotide) includes a nucleic acid sequence encoding a TCR alpha chain, a TCR beta chain, a TCR delta chain, a TCR gamma chain, or any fragment thereof (e.g., variable regions including VDJ or VJ regions, constant regions, transmembrane regions, fragments thereof, combinations thereof, and combinations of fragments thereof). In embodiments, the target nucleic acid includes a nucleic acid sequence encoding a B cell receptor heavy chain, B cell receptor light chain, or any fragment thereof (e.g., variable regions including VDJ or VJ regions, constant regions, transmembrane regions, fragments thereof, combinations thereof, and combinations of fragments thereof). In embodiments, the target nucleic acid includes a CDR3 nucleic acid sequence. In embodiments, the target nucleic acid includes a TCRA gene sequence or a TCRB gene sequence. In embodiments, the target nucleic acid includes a TCRA gene sequence and a TCRB gene sequence. In embodiments, the target nucleic acid includes sequences of various T cell receptor alpha variable genes (TRAV genes), T cell receptor alpha joining genes (TRAJ genes), T cell receptor alpha constant genes (TRAC genes), T cell receptor beta variable genes (TRBV genes), T cell receptor beta diversity genes (TRBD genes), T cell receptor beta joining genes (TRBJ genes), T cell receptor beta constant genes (TRBC genes), T cell receptor gamma variable genes (TRGV genes), T cell receptor gamma joining genes (TRGJ genes), T cell receptor gamma constant genes (TRGC genes), T cell receptor delta variable genes (TRDV genes), T cell receptor delta diversity genes (TRDD genes), T cell receptor delta joining genes (TRDJ genes), or T cell receptor delta constant genes (TRDC genes).

In embodiments, the target polynucleotide includes a cancer-associated gene nucleic acid sequence, a viral nucleic acid sequence, a bacterial nucleic acid sequence, or a fungal nucleic acid sequence. In embodiments, the cancer-associated gene is a nucleic acid sequence identified within The Cancer Genome Atlas Program, accessible at www.cancer.gov/tcga.

In embodiments, the target polynucleotide includes a CD4, CD68, CD20, CD11c, CD8, HLA-DR, Ki67, CD45RO, PanCK, CD3c, CD44, CD45, HLA-A, CD14, CD56, CD57, CD19, CD2, CD1a, CD107a, CD21, Pax5, FOXP3, Granzyme B, CD38, CD39, CD79a, TIGIT, TOX, TP63, S100A4, TFAM, GP100, LaminB1, CK19, CK17, GATA3, SOX2, Bcl2, EpCAM, Caveolin, CD163, CD11b, MPO, CD141, iNOS, PD-1, PD-L1, ICOS, TIM3, LAG3, IDO1, CD40, HLA-E, IFNG, CD69, E-cadherin, CD31, Histone H3, Beta-actin, Podoplanin, SMA, Vimentin, Collagen IV, CD34, Beta-catenin, MMP-9, ZEB1, ASCT2, Na/K ATPase, HK1, LDHA, G6PD, IDH2, GLUT1, pNRF2, ATPA5, SDHA, Citrate Synthase, CPT1A, PARP, BAK, BCL-XL, BAX, BAD, Cytochrome c, LC3B, Beclin-1, H2AX, pRPS6, PCNA, Cyclin D1, HLA-DPB1, LEF1, GAL9, CD138, MC Tryptase, OX40, ZAP70, CD7, C1Qa, CCR6, CD15, AXL, and/or CD227 nucleic acid sequence.

In embodiments, the target polynucleotide can include any polynucleotide of interest. The polynucleotide can include DNA, RNA, peptide nucleic acid, morpholino nucleic acid, locked nucleic acid, glycol nucleic acid, threose nucleic acid, mixtures thereof, and hybrids thereof. In embodiments, the polynucleotide is obtained from one or more source organisms. In some embodiments, the polynucleotide can include a selected sequence or a portion of a larger sequence. In embodiments, sequencing a portion of a polynucleotide or a fragment thereof can be used to identify the source of the polynucleotide. With reference to nucleic acids, polynucleotides and/or nucleotide sequences a “portion,” “fragment” or “region” can be at least 5 consecutive nucleotides, at least 10 consecutive nucleotides, at least 15 consecutive nucleotides, at least 20 consecutive nucleotides, at least 25 consecutive nucleotides, at least 50 consecutive nucleotides, at least 100 consecutive nucleotides, or at least 150 consecutive nucleotides.

In embodiments, the entire sequence of the target polynucleotide is about 1 to 3 kb, and only a portion of that target (e.g., 50 to 100 nucleotides) is sequenced. In embodiments, the target polynucleotide is about 1 to 3 kb. In embodiments, the target polynucleotide is about 1 to 2 kb. In embodiments, the target polynucleotide is about 1 kb. In embodiments, the target polynucleotide is about 2 kb. In embodiments, the target polynucleotide is less than 1 kb. In embodiments, the target polynucleotide is about 500 nucleotides. In embodiments, the target polynucleotide is about 200 nucleotides. In embodiments, the target polynucleotide is about 100 nucleotides. In embodiments, the target polynucleotide is less than 100 nucleotides. In embodiments, the target polynucleotide is about 5 to 50 nucleotides.

In embodiments, the target polynucleotide is an RNA nucleic acid sequence or DNA nucleic acid sequence. In embodiments, the target polynucleotide is an RNA nucleic acid sequence or DNA nucleic acid sequence from the same cell. In embodiments, the target polynucleotide is an RNA nucleic acid sequence. In embodiments, the RNA nucleic acid sequence is stabilized using known techniques in the art. For example, RNA degradation by RNase should be minimized using commercially available solutions, e.g., RNA Later®, RNA Lysis Buffer, or Keratinocyte serum-free medium). In embodiments, the target polynucleotide is messenger RNA (mRNA), transfer RNA (tRNA), micro RNA (miRNA), small interfering RNA (siRNA), small nucleolar RNA (snoRNA), small nuclear RNA (snRNA), Piwi-interacting RNA (piRNA), enhancer RNA (eRNA), or ribosomal RNA (rRNA). In embodiments, the target polynucleotide is pre-mRNA. In embodiments, the target polynucleotide is heterogeneous nuclear RNA (hnRNA). In embodiments, the target polynucleotide is mRNA, tRNA (transfer RNA), rRNA (ribosomal RNA), or noncoding RNA (such as lncRNA (long noncoding RNA)). In embodiments, the target polynucleotides are on different regions of the same RNA nucleic acid sequence. In embodiments, to minimize amplification errors or bias, the target polynucleotide is not reverse transcribed to generate cDNA.

In embodiments, the target polynucleotide includes RNA nucleic acid sequences. In embodiments the target polynucleotide is an RNA transcript. In embodiments the target polynucleotide is a single stranded RNA nucleic acid sequence. In embodiments, the target polynucleotide is an RNA nucleic acid sequence or a DNA nucleic acid sequence (e.g., cDNA). In embodiments, the target polynucleotide is a cDNA target polynucleotide nucleic acid sequence and before step a), the RNA nucleic acid sequence is reverse transcribed to generate the cDNA target polynucleotide nucleic acid sequence. In embodiments, reverse transcription of the RNA nucleic acid is performed with a reverse transcriptase, for example, Tth DNA polymerase or mutants thereof. In embodiments, the target polynucleotide is genomic DNA (gDNA), mitochondrial DNA, chloroplast DNA, episomal DNA, viral DNA, or copy DNA (cDNA). In embodiments, the target polynucleotide is coding RNA such as messenger RNA (mRNA), and non-coding RNA (ncRNA) such as transfer RNA (tRNA), microRNA (miRNA), small nuclear RNA (snRNA), or ribosomal RNA (rRNA). In embodiments, the target polynucleotide is a cancer-associated gene. In embodiments, to minimize amplification errors or bias, the target polynucleotide is not reverse transcribed to generate cDNA.

In embodiments, each oligonucleotide (e.g., one or more oligonucleotides of a plurality of oligonucleotides) includes locked nucleic acids (LNAs), Bis-locked nucleic acids (bisLNAs), twisted intercalating nucleic acids (TINAs), bridged nucleic acids (BNAs), 2′-O-methyl RNA:DNA chimeric nucleic acids, minor groove binder (MGB) nucleic acids, morpholino nucleic acids, C5-modified pyrimidine nucleic acids, peptide nucleic acids (PNAs), or combinations thereof. In embodiments, the first oligonucleotide, the second oligonucleotide, the first oligonucleotide probe, and/or the second oligonucleotide probe includes locked nucleic acids (LNAs), Bis-locked nucleic acids (bisLNAs), twisted intercalating nucleic acids (TINAs), bridged nucleic acids (BNAs), 2′-O-methyl RNA:DNA chimeric nucleic acids, minor groove binder (MGB) nucleic acids, morpholino nucleic acids, C5-modified pyrimidine nucleic acids, peptide nucleic acids (PNAs), or combinations thereof.

In embodiments, each oligonucleotide probe includes one or more locked nucleic acid (LNA) nucleotides. In embodiments, the target hybridization sequence of each oligonucleotide probe includes one or more LNA nucleotides. In embodiments, the first probe hybridization sequence and/or the second probe hybridization sequence of each oligonucleotide probe includes one or more LNA nucleotides. In embodiments, the first target hybridization sequence of the first oligonucleotide and/or the second target hybridization sequence of the second oligonucleotide include one or more LNA nucleotides. In embodiments, the first oligonucleotide probe and/or the second oligonucleotide probe include one or more LNA nucleotides. In embodiments, the oligonucleotide includes 1, 2, 3, 4, 5, 6, 7, 8, 9, or 10 LNAs.

In embodiments, the target hybridization sequence and/or hybridization sequence includes Bis-locked nucleic acids (bisLNAs). In embodiments, the target hybridization sequence and/or hybridization sequence includes twisted intercalating nucleic acids (TINAs). In embodiments, the target hybridization sequence and/or hybridization sequence includes bridged nucleic acids (BNAs). In embodiments, the target hybridization sequence and/or hybridization sequence includes 2′-O-methyl RNA:DNA chimeric nucleic acids. In embodiments, the target hybridization sequence and/or hybridization sequence includes minor groove binder (MGB) nucleic acids. In embodiments, the target hybridization sequence and/or hybridization sequence includes morpholino nucleic acids. Morpholino nucleic acids are synthetic nucleotides that have standard nucleic acid bases (e.g., adenine, guanine, cytosine, and thymine) wherein those bases are bound to methylenemorpholine rings linked through phosphorodiamidate groups instead of phosphates. Morpholino nucleic acids may be referred to as phosphorodiamidate morpholino oligomers (PMOs). In embodiments, the target hybridization sequence and/or hybridization sequence includes C5-modified pyrimidine nucleic acids. In embodiments, the target hybridization sequence and/or hybridization sequence includes peptide nucleic acids (PNAs). In embodiments, the target hybridization sequence and/or hybridization sequence includes from 5′ to 3′ a plurality of synthetic nucleotides (e.g., LNAs) followed by a plurality (e.g., 2 to 5) canonical or native nucleotides (e.g., dNTPs). In embodiments, the target hybridization sequence and/or hybridization sequence includes one or more (e.g., 2 to 5) deoxyuracil nucleobases (dU). In embodiments, the one or more dU nucleobases are at or near the 3′ end of the target hybridization sequence and/or hybridization sequence (e.g., within 5 nucleotides of the 3′ end). In embodiments, the target hybridization sequence and/or hybridization sequence includes from 5′ to 3′ a plurality (e.g., 2 to 5) of phosphorothioate nucleic acids, followed by a plurality of synthetic nucleotides (e.g., LNAs), and subsequently followed by a plurality (e.g., 2 to 5) of canonical nucleobases. In some embodiments, the target hybridization sequence and/or hybridization sequence includes a plurality of canonical nucleobases, wherein the canonical nucleobases terminate (i.e., at the 3′ end) with a deoxyuracil nucleobase (dU).

In embodiments, the target hybridization sequence and/or hybridization sequence includes a plurality of LNAs interspersed throughout the polynucleotide. In embodiments, the target hybridization sequence and/or hybridization sequence includes a plurality of consecutive LNAs (e.g., 2 to 5 LNAs, 5 to 7 LNAs, or 7 to 10 LNAs) throughout the target hybridization sequence and/or hybridization sequence. In embodiments, the entire composition of the target hybridization sequence and/or hybridization sequence includes less than 70%, less than 60%, less than 50%, less than 40%, less than 30%, less than 20%, less than 10%, or less than 5% of LNAs. In embodiments, the entire composition of the target hybridization sequence and/or hybridization sequence includes up to about 70%, up to about 60%, up to about 50%, up to about 40%, up to about 30%, up to about 20%, up to about 10%, or up to about 5% of LNAs. In embodiments, the entire composition of the target hybridization sequence and/or hybridization sequence includes more than 60%, more than 50%, more than 40%, more than 30%, more than 20%, more than 10%, or more than 5% of LNAs. In embodiments, the entire composition of the target hybridization sequence and/or hybridization sequence includes about 5% to about 10%, about 10% to about 20%, about 20% to about 30%, about 30% to about 40%, about 40% to about 50%, about 50% to about 60%, or about 60% to about 70% of LNAs. In embodiments, the entire composition of the target hybridization sequence and/or hybridization sequence includes about 5%, about 10%, about 15%, about 20%, about 25%, about 30%, about 35%, about 40%, about 45%, about 50%, about 55%, about 60%, about 65%, or about 70% of LNAs. In embodiments, the entire composition of the target hybridization sequence and/or hybridization sequence includes about 30%, about 35%, about 40%, about 45%, about 50%, about 55%, about 60%, about 65%, about 70%, about 75%, about 80%, about 85%, about 90%, or about 95% of canonical dNTPs. In embodiments, the entire composition of the target hybridization sequence and/or hybridization sequence includes less than 95%, less than 90%, less than 80%, less than 70%, less than 60%, less than 50%, less than 40%, or less than 30% of canonical dNTPs. In embodiments, the entire composition of the target hybridization sequence and/or hybridization sequence includes up to about 95%, up to about 90%, up to about 80%, up to about 70%, up to about 60%, up to about 50%, up to about 40%, or up to about 30% of canonical dNTPs. In embodiments, the entire composition of the target hybridization sequence and/or hybridization sequence includes more than 90%, more than 80%, more than 70%, more than 60%, more than 50%, more than 40%, or more than 30% of canonical dNTPs.

In embodiments, the entire composition of the target hybridization sequence and/or hybridization sequence includes about 70% of LNAs and about 30% of canonical dNTPs. In embodiments, the entire composition of the target hybridization sequence and/or hybridization sequence includes about 65% of LNAs and about 35% of canonical dNTPs. In embodiments, the entire composition of the target hybridization sequence and/or hybridization sequence includes about 60% of LNAs and about 40% of canonical dNTPs. In embodiments, the entire composition of the target hybridization sequence and/or hybridization sequence includes about 55% of LNAs and about 45% of canonical dNTPs. In embodiments, the entire composition of the target hybridization sequence and/or hybridization sequence includes about 50% of LNAs and about 50% of canonical dNTPs. In embodiments, the entire composition of the target hybridization sequence and/or hybridization sequence includes about 45% of LNAs and about 55% of canonical dNTPs. In embodiments, the entire composition of the target hybridization sequence and/or hybridization sequence includes about 40% of LNAs and about 60% of canonical dNTPs. In embodiments, the entire composition of the target hybridization sequence and/or hybridization sequence includes about 35% of LNAs and about 65% of canonical dNTPs. In embodiments, the entire composition of the target hybridization sequence and/or hybridization sequence includes about 30% of LNAs and about 70% of canonical dNTPs. In embodiments, the entire composition of the target hybridization sequence and/or hybridization sequence includes about 25% of LNAs and about 75% of canonical dNTPs. In embodiments, the entire composition of the target hybridization sequence and/or hybridization sequence includes about 20% of LNAs and about 80% of canonical dNTPs. In embodiments, the entire composition of the target hybridization sequence and/or hybridization sequence includes about 15% of LNAs and about 85% of canonical dNTPs. In embodiments, the entire composition of the target hybridization sequence and/or hybridization sequence includes about 10% of LNAs and about 90% of canonical dNTPs. In embodiments, the entire composition of the target hybridization sequence and/or hybridization sequence includes about 5% of LNAs and about 95% of canonical dNTPs.

In embodiments, each oligonucleotide includes a blocking moiety at a 3′ end (e.g., at the 3′ end of each oligonucleotide of a plurality of oligonucleotides). In embodiments, the first oligonucleotide and/or the second oligonucleotide includes a blocking moiety at the 3′ end. In embodiments, the first oligonucleotide and the second oligonucleotide include a blocking moiety at the 3′ end. In embodiments, the first oligonucleotide probe includes a blocking moiety at the 3′ end. In embodiments, a terminal nucleotide of the first oligonucleotide includes a blocking moiety. In embodiments, a terminal nucleotide of the second oligonucleotide includes a blocking moiety. In embodiments, a terminal nucleotide of the first oligonucleotide probe includes a blocking moiety. In embodiments, the blocking moiety is reversible. In embodiments, the blocking moiety is irreversible. In embodiments, the blocking moiety at the 3′ end (e.g., the 3′ blocking moiety) includes a reversible terminator. In embodiments, the 3′ blocking moiety includes a dideoxynucleotide triphosphate (e.g., a ddNTP).

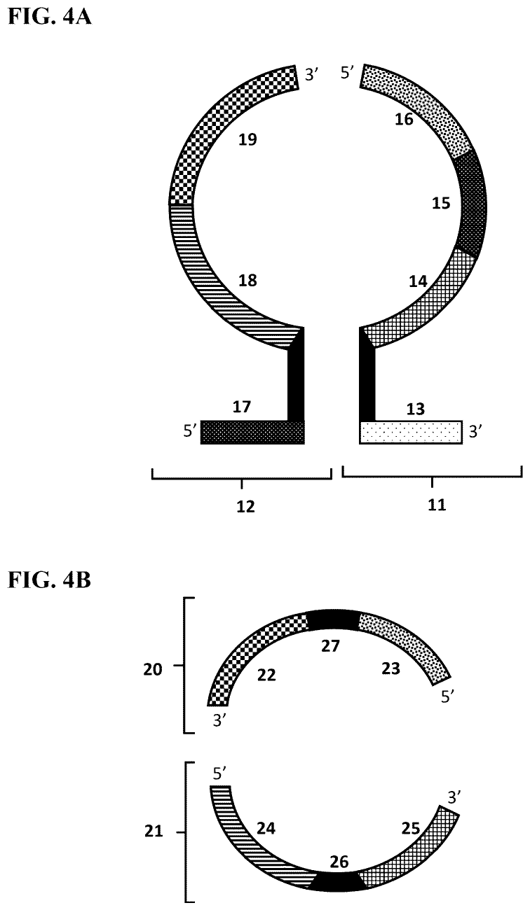

In embodiments, the first oligonucleotide (e.g., as illustrated in FIG. 4A; 11) includes, from 3′ to 5′, a first target hybridization sequence (13), a first hybridization sequence (14; e.g., a sequence complementary to a 3′ end of a second oligonucleotide probe), a barcode sequence (15), and a second hybridization sequence (16; e.g., a sequence complementary to a 5′ end of a first oligonucleotide probe). In embodiments, the first oligonucleotide includes, from 5′ to 3′, a sequence complementary to a 5′ end of a first oligonucleotide probe, a barcode sequence, a sequence complementary to a 3′ end of a second oligonucleotide probe, and a first target hybridization sequence. In embodiments, the second oligonucleotide (e.g., as illustrated in FIG. 4A; 12) includes, from 5′ to 3′, a second target hybridization sequence (17), a third hybridization sequence (18; e.g., a sequence complementary to a 5′ end of a second oligonucleotide probe), and a fourth hybridization sequence (19; e.g., a sequence complementary to a 3′ end of a first oligonucleotide probe). In embodiments, the second oligonucleotide includes, from 5′ to 3′, a second target hybridization sequence, a sequence complementary to a 5′ end of a second oligonucleotide probe, and a sequence complementary to a 3′ end of a first oligonucleotide probe. In embodiments, the first oligonucleotide probe includes (e.g., as illustrated in FIG. 4B; 20), from 5′ to 3′, a second hybridization sequence complement (23), an optional linker sequence (27), and a fourth hybridization sequence complement (22). In embodiments, the first oligonucleotide probe includes, from 5′ to 3′, a sequence complementary to a 5′ end of the first oligonucleotide, an optional linker sequence, and a sequence complementary to a 3′ end of the second oligonucleotide. In embodiments, the second oligonucleotide probe includes (e.g., as illustrated in FIG. 4B; 21), from 5′ to 3′, a third hybridization sequence complement (24), an optional linker sequence (26), and a first hybridization sequence complement (25). In embodiments, the second oligonucleotide probe includes, from 5′ to 3′, a sequence complementary to a sequence downstream of the second target hybridization sequence of the second oligonucleotide, an optional linker sequence, and a sequence complementary to a sequence upstream of the first target hybridization sequence of the first oligonucleotide. In embodiments, the second oligonucleotide further includes a blocking moiety at the 3′ end. In embodiments, the first oligonucleotide further includes a blocking moiety at the 3′ end. In embodiments, the first oligonucleotide probe further includes a blocking moiety at the 3′ end. In embodiments, the second oligonucleotide probe further includes a blocking moiety at the 3′ end.

In embodiments, the first oligonucleotide probe and the second oligonucleotide probe hybridize to the first oligonucleotide and the second oligonucleotide such that the duplexed first hybridization sequence and second hybridization sequence flank the barcode sequence of the first oligonucleotide. In embodiments, the first oligonucleotide probe and the second oligonucleotide probe hybridize to the first oligonucleotide and the second oligonucleotide such that the duplexed third hybridization sequence and fourth hybridization sequence are separated by a nick (e.g., the duplex third and fourth hybridization sequences are separated by a nick that may be covalently sealed by, for example, a DNA ligase). In embodiments, the first oligonucleotide probe and the second oligonucleotide probe hybridize to the first oligonucleotide and the second oligonucleotide such that the duplexed third hybridization sequence and fourth hybridization sequence are separated by a 1, 2, 3, 4, or 5 nucleotide gap (e.g., the duplex third and fourth hybridization sequences are separated by gap that may be filled with a polymerase and covalently sealed by, for example, a DNA ligase). In some embodiments, the gap between the duplexed third and second oligonucleotide probes is more than 5 nucleotides.

In embodiments, the barcode sequence is associated with the target polynucleotide (e.g., the barcode sequence of the oligonucleotide identifies the target polynucleotide to which the oligonucleotide is hybridized to). In embodiments, the barcode sequence is associated with the target hybridization sequence of the oligonucleotide probe (e.g., the barcode sequence identifies the sequence of the target polynucleotide that is complementary to the target hybridization sequence of the oligonucleotide). In embodiments, the barcode sequence is associated with both the target polynucleotide and the target hybridization sequence of the oligonucleotide (e.g., the barcode sequence corresponds to, and identifies, the sequence of the target polynucleotide that is complementary to the target hybridization sequence of the oligonucleotide, as well as the target polynucleotide itself). In embodiments, the target sequence is represented by a barcode sequence.

In embodiments, each of the known set of barcode sequences is associated with a target hybridization sequence from a known set of target hybridization sequences. In embodiments, the first barcode sequence is associated with the first target hybridization sequence, and wherein the second barcode sequence is associated with the second target hybridization sequence. For example, an oligonucleotide probe includes a barcode sequence (e.g., a barcode sequence selected from a known set of barcode sequences) that is associated with a target hybridization sequence (e.g., a sequence complementary to a sequence of a target polynucleotide) of the oligonucleotide, such that the barcode sequence is also associated with the sequence of the target polynucleotide. In embodiments, the first barcode sequence is associated with a first sequence of the target polynucleotide. In embodiments, the second barcode sequence is associated with a first sequence of the target polynucleotide. In embodiments, the first barcode sequence is different than the second barcode sequence.

In embodiments, the targets are proteins or carbohydrates. In embodiments, the targets are proteins. In embodiments, the targets are carbohydrates. In embodiments when the target are proteins and/or carbohydrates, the method includes contacting the proteins with a specific binding reagent, wherein the specific binding reagent includes an oligonucleotide barcode (e.g., the target polynucleotide is attached to the specific binding reagent). In embodiments, the specific binding reagent includes an antibody, single-chain Fv fragment (scFv), antibody fragment-antigen binding (Fab), or an aptamer. In embodiments, the specific binding reagent is a peptide, a cell penetrating peptide, an aptamer, a DNA aptamer, an RNA aptamer, an antibody, an antibody fragment, a light chain antibody fragment, a single-chain variable fragment (scFv), a lipid, a lipid derivative, a phospholipid, a fatty acid, a triglyceride, a glycerolipid, a glycerophospholipid, a sphingolipid, a saccharolipid, a polyketide, a polylysine, polyethyleneimine, diethylaminocthyl (DEAE)-dextran, cholesterol, or a sterol moiety. In embodiments, the specific binding reagent interacts (e.g., contacts, or binds) with one or more specific binding reagents on the cell surface. Carbohydrate-specific antibodies are known in the art, see for example Kappler, K., Hennet, T. Genes Immun 21, 224-239 (2020) In embodiments, the target polynucleotide is polynucleotide attached to a specific binding reagent. In embodiments, the specific binding reagent is an antibody, single-chain Fv fragment (scFv), antibody fragment-antigen binding (Fab), or an aptamer.

In embodiments, the target polynucleotide is attached to a specific binding reagent (e.g., an antibody) via a linker (e.g., a bioconjugate linker). In embodiments, the target polynucleotide is attached to the specific binding reagent via a linker formed by reacting a first bioconjugate reactive moiety (e.g., the bioconjugate reactive moiety includes an amine moiety, aldehyde moiety, alkyne moiety, azide moiety, carboxylic acid moiety, dibenzocyclooctyne (DBCO) moiety, tetrazine moiety, epoxy moiety, isocyanate moiety, furan moiety, maleimide moiety, thiol moiety, or transcyclooctene (TCO) moiety) with a second bioconjugate reactive moiety). In embodiments, the target polynucleotide includes a barcode, wherein the barcode is a known sequence associated with the specific binding reagent. In embodiments, the barcode is at least 5, 6, 7, 8, 9, 10, 11, 12, 13, 14, or 15 nucleotides in length. In embodiments, the barcode is 5, 6, 7, 8, 9, 10, 11, 12, 13, 14, or 15 nucleotides in length.

Specific antibodies tagged with known oligonucleotide sequences can be synthesized by using bifunctional crosslinkers reactive towards thiol (via maleimide) and amine (via NHS) moieties. For example, a 5′-thiol-modified oligonucleotide could be conjugated to a crosslinker via maleimide chemistry and purified. The oligos with a 5′-NHS-ester would then be added to a solution of antibodies and reacted with amine residues on the antibodies surface to generate tagged antibodies capable of binding analytes with target epitopes. These tagged antibodies include oligonucleotide sequence(s). The one or more oligonucleotide sequences may include a barcode, binding sequences (e.g., primer binding sequence or sequences complementary to hybridization pads), and/or unique molecular identifier (UMI) sequences.

In embodiments, specific binding entails a binding affinity, expressed as a K_{D} (such as a K_{D} measured by surface plasmon resonance at an appropriate temperature, such as 37° C.). In embodiments, the K_{D} of a specific binding interaction is less than about 100 nM, 50 nM, 10 nM, 1 nM, 0.05 nM, or lower. In embodiments, the K_{D} of a specific binding interaction is about 0.01-100 nM, 0.1-50 nM, or 1-10 nM. In embodiments, the K_{D} of a specific binding interaction is less than 10 nM. The binding affinity of an antibody can be readily determined by one of ordinary skill in the art (for example, by Scatchard analysis). A variety of immunoassay formats can be used to select antibodies specifically immunoreactive with a particular antigen. For example, solid-phase ELISA immunoassays are routinely used to select monoclonal antibodies specifically immunoreactive with an analyte. See Harlow and Lane, ANTIBODIES: A LABORATORY MANUAL, Cold Springs Harbor Publications, New York, (1988) for a description of immunoassay formats and conditions that can be used to determine specific immunoreactivity. Typically a specific or selective reaction will be at least twice background signal to noise and more typically more than 10 to 100 times greater than background.

In embodiments, the methods and compositions described herein are utilized to analyze the various sequences of TCRs and BCRs from immune cells, for example various clonotypes. In embodiments, the target nucleic acid includes a nucleic acid sequence encoding a TCR alpha chain, a TCR beta chain, a TCR delta chain, a TCR gamma chain, or any fragment thereof (e.g., variable regions including VDJ or VJ regions, constant regions, transmembrane regions, fragments thereof, combinations thereof, and combinations of fragments thereof). In embodiments, the target nucleic acid includes a nucleic acid sequence encoding a B cell receptor heavy chain, B cell receptor light chain, or any fragment thereof (e.g., variable regions including VDJ or VJ regions, constant regions, transmembrane regions, fragments thereof, combinations thereof, and combinations of fragments thereof). In embodiments, the target nucleic acid includes a CDR3 nucleic acid sequence. In embodiments, the target nucleic acid includes a TCRA gene sequence or a TCRB gene sequence. In embodiments, the target nucleic acid includes a TCRA gene sequence and a TCRB gene sequence. In embodiments, the target nucleic acid includes sequences of various T cell receptor alpha variable genes (TRAV genes), T cell receptor alpha joining genes (TRAJ genes), T cell receptor alpha constant genes (TRAC genes), T cell receptor beta variable genes (TRBV genes), T cell receptor beta diversity genes (TRBD genes), T cell receptor beta joining genes (TRBJ genes), T cell receptor beta constant genes (TRBC genes), T cell receptor gamma variable genes (TRGV genes), T cell receptor gamma joining genes (TRGJ genes), T cell receptor gamma constant genes (TRGC genes), T cell receptor delta variable genes (TRDV genes), T cell receptor delta diversity genes (TRDD genes), T cell receptor delta joining genes (TRDJ genes), or T cell receptor delta constant genes (TRDC genes).

RNA, including mRNA, is highly susceptible to degradation upon exposure to one or more RNAses. RNAses are present in a wide range of locations, including water, many reagents, laboratory equipment and surfaces, skin, and mucous membranes. Working with RNA often requires preparing an RNAse-free environment and materials, as well as taking precautions to avoid introducing RNAses into an RNAse-free environment. These precautions include, but are not limited to, cleaning surfaces with an RNAse cleaning product (e.g., RNASEZAP™ and other commercially available products or 0.5% sodium dodecyl sulfate [SDS] followed by 3% H₂O₂); using a designated workspace, materials, and equipment (e.g., pipets, pipet tips); using barrier tips; baking designated glassware (e.g., 300° C. for 2 hours) prior to use; treating enzymes, reagents, and other solutions (e.g., with diethyl pyrocarbonate [DEPC] or dimethyl pyrocarbonate [DMPC]) or using commercially available, certified RNAse-free water or solutions, or ultrafiltered water (e.g., for Tris-based solutions); including an RNAse inhibitor while avoiding temperatures or denaturing conditions that could deactivate the inhibitor); and wearing clean gloves (while avoiding contaminated surfaces) and a clean lab coat.

In embodiments, the cell forms part of a tissue in situ. In embodiments, the cell is an isolated single cell. In embodiments, the cell is a prokaryotic cell. In embodiments, the cell is a eukaryotic cell. In embodiments, the cell is a bacterial cell (e.g., a bacterial cell or bacterial spore), a fungal cell (e.g., a fungal spore), a plant cell, or a mammalian cell. In embodiments, the cell is a stem cell. In embodiments, the stem cell is an embryonic stem cell, a tissue-specific stem cell, a mesenchymal stem cell, or an induced pluripotent stem cell. In embodiments, the cell is an endothelial cell, muscle cell, myocardial, smooth muscle cell, skeletal muscle cell, mesenchymal cell, epithelial cell; hematopoietic cell, such as lymphocytes, including T cell, e.g., (Th1 T cell, Th2 T cell, ThO T cell, cytotoxic T cell); B cell, pre-B cell; monocytes; dendritic cell; neutrophils; or a macrophage. In embodiments, the cell is a stem cell, an immune cell, a cancer cell (e.g., a circulating tumor cell or cancer stem cell), a viral-host cell, or a cell that selectively binds to a desired target. In embodiments, the cell includes a T cell receptor gene sequence, a B cell receptor gene sequence, or an immunoglobulin gene sequence. In embodiments, the cell includes a Toll-like receptor (TLR) gene sequence. In embodiments, the cell includes a gene sequence corresponding to an immunoglobulin light chain polypeptide and a gene sequence corresponding to an immunoglobulin heavy chain polypeptide. In embodiments, the cell is a genetically modified cell. In embodiments, the cell is a circulating tumor cell or cancer stem cell.

In embodiments, the cell is a prokaryotic cell. In embodiments, the cell is a bacterial cell. In embodiments, the bacterial cell is a Bacteroides, Clostridium, Faecalibacterium, Eubacterium, Ruminococcus, Peptococcus, Peptostreptococcus, or Bifidobacterium cell. In embodiments, the bacterial cell is a Bacteroides fragilis, Bacteroides melaninogenicus, Bacteroides oralis, Enterococcus faecalis, Escherichia coli, Enterobacter sp., Klebsiella sp., Bifidobacterium bifidum, Staphylococcus aureus, Lactobacillus, Clostridium perfringens, Proteus mirabilis, Clostridium tetani, Clostridium septicum, Pseudomonas aeruginosa, Salmonella enterica, Faecalibacterium prausnitzii, Peptostreptococcus sp., or Peptococcus sp. cell. In embodiments, the cell is a fungal cell. In embodiments, the fungal cell is a Candida, Saccharomyces, Aspergillus, Penicillium, Rhodotorula, Trametes, Pleospora, Sclerotinia, Bullera, or a Galactomyces cell.

In embodiments, the cell is a viral-host cell. A “viral-host cell” is used in accordance with its ordinary meaning in virology and refers to a cell that is infected with a viral genome (e.g., viral DNA or viral RNA). The cell, prior to infection with a viral genome, can be any cell that is susceptible to viral entry. In embodiments, the viral-host cell is a lytic viral-host cell. In embodiments, the viral-host cell is capable of producing viral protein. In embodiments, the viral-host cell is a lysogenic viral-host cell. In embodiments, the cell is a viral-host cell including a viral nucleic acid sequence, wherein the viral nucleic acid sequence is from a Hepadnaviridae, Adenoviridae, Herpesviridae, Poxviridae, Parvoviridae, Reoviridae, Coronaviridae, Retroviridae virus.

In embodiments, the cell is an adherent cell (e.g., epithelial cell, endothelial cell, or neural cell). Adherent cells are usually derived from tissues of organs and attach to a substrate (e.g., epithelial cells adhere to an extracellular matrix coated substrate via transmembrane adhesion protein complexes). Adherent cells typically require a substrate, e.g., tissue culture plastic, which may be coated with extracellular matrix (e.g., collagen and laminin) components to increase adhesion properties and provide other signals needed for growth and differentiation. In embodiments, the cell is a neuronal cell, an endothelial cell, epithelial cell, germ cell, plasma cell, a muscle cell, peripheral blood mononuclear cell (PBMC), a myocardial cell, or a retina cell. In embodiments, the cell is a suspension cell (e.g., a cell free-floating in the culture medium, such a lymphoblast or hepatocyte). In embodiments, the cell is a glial cell (e.g., astrocyte, radial glia), pericyte, or stem cell (e.g., a neural stem cell). In embodiments, the cell is a neuronal cell. In embodiments, the cell is an endothelial cell. In embodiments, the cell is an epithelial cell. In embodiments, the cell is a germ cell. In embodiments, the cell is a plasma cell. In embodiments, the cell is a muscle cell. In embodiments, the cell is a peripheral blood mononuclear cell (PBMC). In embodiments, the cell is a myocardial cell. In embodiments, the cell is a retina cell. In embodiments, the cell is a lymphoblast. In embodiments, the cell is a hepatocyte. In embodiments, the cell is a glial cell. In embodiments, the cell is an astrocyte. In embodiments, the cell is a radial glia. In embodiments, the cell is a pericyte. In embodiments, the cell is a stem cell. In embodiments, the cell is a neural stem cell.

In embodiments, the cell is bound to a known antigen. In embodiments, the cell is a cell that selectively binds to a desired target, wherein the target is an antibody, or antigen binding fragment, an aptamer, affimer, non-immunoglobulin scaffold, small molecule, or genetic modifying agent. In embodiments, the cell is a leukocyte (i.e., a white-blood cell). In embodiments, leukocyte is a granulocyte (neutrophil, cosinophil, or basophil), monocyte, or lymphocyte (T cells and B cells). In embodiments, the cell is a lymphocyte. In embodiments, the cell is a T cell, an NK cell, or a B cell.

In embodiments, the cell is an immune cell. In embodiments, the immune cell is a granulocyte, a mast cell, a monocyte, a neutrophil, a dendritic cell, or a natural killer (NK) cell. In embodiments, the immune cell is an adaptive cell, such as a T cell, NK cell, or a B cell. In embodiments, the cell includes a T cell receptor gene sequence, a B cell receptor gene sequence, or an immunoglobulin gene sequence. In embodiments, the immune cell is a granulocyte. In embodiments, the immune cell is a mast cell. In embodiments, the immune cell is a monocyte. In embodiments, the immune cell is a neutrophil. In embodiments, the immune cell is a dendritic cell. In embodiments, the immune cell is a natural killer (NK) cell. In embodiments, the immune cell is a T cell. In embodiments, the immune cell is a B cell. In embodiments, the cell includes a T cell receptor gene sequence. In embodiments, the cell includes a B cell receptor gene sequence. In embodiments, the cell includes an immunoglobulin gene sequence. In embodiments, the plurality of target nucleic acids includes non-contiguous regions of a nucleic acid molecule. In embodiments, the non-contiguous regions include regions of a VDJ recombination of a B cell or T cell.

In embodiments, the cell is a cancer cell. In embodiments, the cancer is lung cancer, colorectal cancer, skin cancer, colon cancer, pancreatic cancer, breast cancer, cervical cancer, lymphoma, leukemia, or a cancer associated with aberrant K-Ras, aberrant APC, aberrant Smad4, aberrant p53, or aberrant TGFβ. In embodiments, the cancer cell includes a ERBB2, KRAS, TP53, PIK3CA, or FGFR2 gene. In embodiments, the cancer cell includes a HER2 gene. In embodiments, the cancer cell includes a cancer-associated gene (e.g., an oncogene associated with kinases and genes involved in DNA repair) or a cancer-associated biomarker. A “biomarker” is a substance that is associated with a particular characteristic, such as a disease or condition. A change in the levels of a biomarker may correlate with the risk or progression of a disease or with the susceptibility of the disease to a given treatment. In embodiments, the cancer is Acute Myeloid Leukemia, Adrenocortical Carcinoma, Bladder Urothelial Carcinoma, Breast Ductal Carcinoma, Breast Lobular Carcinoma, Cervical Carcinoma, Cholangiocarcinoma, Colorectal Adenocarcinoma, Esophageal Carcinoma, Gastric Adenocarcinoma, Glioblastoma Multiforme, Head and Neck Squamous Cell Carcinoma, Hepatocellular Carcinoma, Kidney Chromophobe Carcinoma, Kidney Clear Cell Carcinoma, Kidney Papillary Cell Carcinoma, Lower Grade Glioma, Lung Adenocarcinoma, Lung Squamous Cell Carcinoma, Mesothelioma, Ovarian Serous Adenocarcinoma, Pancreatic Ductal Adenocarcinoma, Paraganglioma & Pheochromocytoma, Prostate Adenocarcinoma, Sarcoma, Skin Cutaneous Melanoma, Testicular Germ Cell Cancer, Thymoma, Thyroid Papillary Carcinoma, Uterine Carcinosarcoma, Uterine Corpus Endometrioid Carcinoma, or Uveal Melanoma. In embodiments, the cancer-associated gene is a nucleic acid sequence identified within The Cancer Genome Atlas Program, accessible at www.cancer.gov/tcga.

In embodiments, the cancer-associated biomarker is MDC, NME-2, KGF, PIGF, Flt-3L, HGF, MCP1, SAT-1, MIP-1-b, GCLM, OPG, TNF RII, VEGF-D, ITAC, MMP-10, GPI, PPP2R4, AKRIB1, Amy1A, MIP-1b, P-Cadherin, or EPO. In embodiments, the cancer-associated gene is a AKT1, AKT2, AKT3, ALK, AR, ARAF, ARID1A, ATM, ATR, ATRX, AXL, BAP1, BRAF, BRCA1, BRCA2, BTK, CBL, CCND1, CCND2, CCND3, CCNE1, CDK12, CDK2, CDK4, CDK6, CDKN1B, CDKN2A, CDKN2B, CHEK1, CHEK2, CREBBP, CSF1R, CTNNB1, DDR2, EGFR, ERBB2, ERBB3, ERBB4, ERCC2, ERG, ESR1, ETV1, ETV4, ETV5, EZH2, FANCA, FANCD2, FANCI, FBXW7, FGF19, FGF3, FGFR1, FGFR2, FGFR3, FGFR4, FGR, FLT3, FOXL2, GATA2, GNA11, GNAQ, GNAS, H3F3A, HIST1H3B, HNF1A, HRAS, IDH1, IDH2, IGF1R, JAK1, JAK2, JAK3, KDR, KIT, KNSTRN, KRAS, MAGOH, MAP2K1, MAP2K2, MAP2K4, MAPK1, MAX, MDM2, MDM4, MED12, MET, MLH1, MREI1A, MSH2, MSH6, MTOR, MYB, MYBLI, MYC, MYCL, MYCN, MYD88, NBN, NF1, NF2, NFE2L2, NOTCH1, NOTCH2, NOTCH3, NOTCH4, NRAS, NRG1, NTRK1, NTRK2, NTRK3, NUTM1, PALB2, PDGFRA, PDGFRB, PIK3CA, PIK3CB, PIK3R1, PMS2, POLE, PPARG, PPP2RIA, PRKACA, PRKACB, PTCH1, PTEN, PTPN11, RAC1, RAD50, RAD51, RAD51B, RAD51C, RAD51D, RAF1, RB1, RELA, RET, RHEB, RHOA, RICTOR, RNF43, ROS1, RSPO2, RSPO3, SETD2, SF3B1, SLX4, SMAD4, SMARCA4, SMARCB1, SMO, SPOP, SRC, STAT3, STK11, TERT, TOP1, TP53, TSC1, TSC2, U2AF1, or XPO1 gene. In embodiments, the cancer-associated gene is a ABL1, AKT1, ALK, APC, ATM, BRAF, CDH1, CDKN2A, CSF1R, CTNNB1, EGFR, ERBB2, ERBB4, EZH2, FBXW7, FGFR1, FGFR2, FGFR3, FLT3, GNA11, GNAQ, GNAS, HNF1A, HRAS, IDH1, IDH2, JAK2, JAK3, KDR, KIT, KRAS, MET, MLH1, MPL, NOTCH1, NPM1, NRAS, PDGFRA, PIK3CA, PTEN, PTPN11, RB1, RET, SMAD4, SMARCB1, SMO, SRC, STK11, TP53, or VHL gene. In embodiments, the cell is a cell (e.g., a T cell) within a tumor. In embodiments, the cell is a non-allogenic cell (i.e., native cell to the subject) within a tumor. In embodiments, the cell is a tumor infiltrating lymphocyte (TIL). In embodiments, the cell is an allogenic cell. In embodiments, the cell is a circulating tumor cell.

In embodiments, the cell in situ is obtained from a subject (e.g., human or animal tissue). Once obtained, the cell is placed in an artificial environment in plastic or glass containers supported with specialized medium containing essential nutrients and growth factors to support proliferation. In embodiments, the cell is permeabilized and immobilized to a solid support surface. In embodiments, the cell is permeabilized and immobilized to an array (i.e., to discrete locations arranged in an array). In embodiments, the cell is immobilized to a solid support surface. In embodiments, the surface includes a patterned surface (e.g., suitable for immobilization of a plurality of cells in an ordered pattern. The discrete regions of the ordered pattern may have defined locations in a regular array, which may correspond to a rectilinear pattern, circular pattern, hexagonal pattern, or the like. These discrete regions are separated by interstitial regions. As used herein, the term “interstitial region” refers to an area in a substrate or on a surface that separates other areas of the substrate or surface. In embodiments, a plurality of cells are immobilized on a patterned surface that have a mean or median separation from one another of about 10-20 μm. In embodiments, a plurality of cells are immobilized on a patterned surface that have a mean or median separation from one another of about 10-20; 10-50; or 100 μm. In embodiments, a plurality of cells are arrayed on a substrate. In embodiments, a plurality of cells are immobilized in a 96-well microplate having a mean or median well-to-well spacing of about 8 mm to about 12 mm (e.g., about 9 mm). In embodiments, a plurality of cells are immobilized in a 384-well microplate having a mean or median well-to-well spacing of about 3 mm to about 6 mm (e.g., about 4.5 mm).

In embodiments, the cell is attached to the substrate via a bioconjugate reactive linker. In embodiments, the cell is attached to the substrate via a specific binding reagent. In embodiments, the specific binding reagent includes an antibody, single-chain Fv fragment (scFv), antibody fragment-antigen binding (Fab), or an aptamer. In embodiments, the specific binding reagent includes an antibody, or antigen binding fragment, an aptamer, affimer, or non-immunoglobulin scaffold. In embodiments, the specific binding reagent is a peptide, a cell penetrating peptide, an aptamer, a DNA aptamer, an RNA aptamer, an antibody, an antibody fragment, a light chain antibody fragment, a single-chain variable fragment (scFv), a lipid, a lipid derivative, a phospholipid, a fatty acid, a triglyceride, a glycerolipid, a glycerophospholipid, a sphingolipid, a saccharolipid, a polyketide, a polylysine, polyethyleneimine, diethylaminoethyl (DEAE)-dextran, cholesterol, or a sterol moiety. Substrates may be prepared for selective capture of particular cells. For example, a substrate containing a plurality of bioconjugate reactive moieties or a plurality of specific binding reagents, optionally in an ordered pattern, contacts a plurality of cells. Only cells containing complementary bioconjugate reactive moieties or complementary specific binding reagents are capable of reacting, and thus adhering, to the substrate.

In embodiments, the cell is permeabilized. In embodiments, the methods are performed in situ on isolated cells or in tissue sections that have been prepared according to methodologies known in the art. Methods for permeabilization and fixation of cells and tissue samples are known in the art, as exemplified by Cremer et al., The Nucleus: Volume 1: Nuclei and Subnuclear Components, R. Hancock (ed.) 2008; and Larsson et al., Nat. Methods (2010) 7:395-397, the content of each of which is incorporated herein by reference in its entirety. In embodiments, the cell is cleared (e.g., digested) of proteins, lipids, or proteins and lipids. In embodiments, the method includes digesting the cell by contacting the cell with an endopeptidase.

In embodiments, the cell is immobilized to a substrate. The cell may have been cultured on the surface, or the cell may have been initially cultured in suspension and then fixed to the surface. Substrates can be two- or three-dimensional and can include a planar surface (e.g., a glass slide). A substrate can include glass (e.g., controlled pore glass (CPG)), quartz, plastic (such as polystyrene (low cross-linked and high cross-linked polystyrene), polycarbonate, polypropylene and poly(methymethacrylate)), acrylic copolymer, polyamide, silicon, metal (e.g., alkanethiolate-derivatized gold), cellulose, nylon, latex, dextran, gel matrix (e.g., silica gel), polyacrolein, or composites. In embodiments, the substrate includes a polymeric coating, optionally containing bioconjugate reactive moieties capable of affixing the sample. Suitable three-dimensional substrates include, for example, spheres, microparticles, beads, membranes, slides, plates, micromachined chips, tubes (e.g., capillary tubes), microwells, microfluidic devices, channels, filters, or any other structure suitable for anchoring a sample. In embodiments, the substrate is not a flow cell. In embodiments, the substrate includes a polymer matrix material (e.g., polyacrylamide, cellulose, alginate, polyamide, cross-linked agarose, cross-linked dextran or cross-linked polyethylene glycol), which may be referred to herein as a “matrix”, “synthetic matrix”, “exogenous polymer” or “exogenous hydrogel”. In embodiments, a matrix may refer to the various components and organelles of a cell, for example, the cytoskeleton (e.g., actin and tubulin), endoplasmic reticulum, Golgi apparatus, vesicles, etc. In embodiments, the matrix is endogenous to a cell. In embodiments, the matrix is exogenous to a cell. In embodiments, the matrix includes both the intracellular and extracellular components of a cell. In embodiments, polynucleotide primers may be immobilized on a matrix including the various components and organelles of a cell. Immobilization of polynucleotide primers on a matrix of cellular components and organelles of a cell is accomplished as described herein, for example, through the interaction/reaction of complementary bioconjugate reactive moieties. In embodiments, the exogenous polymer may be a matrix or a network of extracellular components that act as a point of attachment (e.g., act as an anchor) for the cell to a substrate.”

In embodiments, the cell is exposed to paraformaldehyde (i.e., by contacting the cell with paraformaldehyde). Any suitable permeabilization and fixation technologies can be used for making the cell available for the detection methods provided herein. In embodiments the method includes affixing single cells or tissues to a transparent substrate. Exemplary tissue include those from skin tissue, muscle tissue, bone tissue, organ tissue and the like. In embodiments, the method includes immobilizing the cell in situ to a substrate and permeabilized for delivering probes, enzymes, nucleotides and other components required in the reactions. In embodiments, the cell includes many cells from a tissue section in which the original spatial relationships of the cells are retained. In embodiments, the cell in situ is within a Formalin-Fixed Paraffin-Embedded (FFPE) sample. In embodiments, the cell is subjected to paraffin removal methods, such as methods involving incubation with a hydrocarbon solvent, such as xylene or hexane, followed by two or more washes with decreasing concentrations of an alcohol, such as ethanol. The cell may be rehydrated in a buffer, such as PBS, TBS or MOPs. In embodiments, the FFPE sample is incubated with xylene and washed using ethanol to remove the embedding wax, followed by treatment with Proteinase K to permeabilized the tissue. In embodiments, the cell is fixed with a chemical fixing agent. In embodiments, the chemical fixing agent is formaldehyde or glutaraldehyde. In embodiments, the chemical fixing agent includes both formaldehyde and glutaraldehyde. In embodiments, the chemical fixing agent is glyoxal or dioxolane. In embodiments, the chemical fixing agent includes one or more of ethanol, methanol, 2-propanol, acetone, and glyoxal. In embodiments, the chemical fixing agent includes formalin, Greenfix®, Greenfix® Plus, UPM, CyMol®, HOPE®, CytoSkelFix™, F-Solv®, FineFIX® RCL2/KINFix, UMFIX, Glyo-Fixx®, Histochoice®, or PAXgene®. In embodiments, the cell is fixed within a synthetic three-dimensional matrix (e.g., polymeric material). In embodiments, the synthetic matrix includes polymeric-crosslinking material. In embodiments, the material includes polyacrylamide, poly-ethylene glycol (PEG), poly(acrylate-co-acrylic acid) (PAA), or Poly(N-isopropylacrylamide) (NIPAM).

In embodiments the cell is lysed to release nucleic acid or other materials from the cells. For example, the cells may be lysed using reagents (e.g., a surfactant such as Triton-X or SDS, an enzyme such as lysozyme, lysostaphin, zymolase, cellulase, mutanolysin, glycanases, proteases, mannase, proteinase K, etc.) or a physical lysing mechanism a physical condition (e.g., ultrasound, ultraviolet light, mechanical agitation, etc.). The cells may release, for instance, DNA, RNA, mRNA, proteins, or enzymes. The cells may arise from any suitable source. For instance, the cells may be any cells for which nucleic acid from the cells is desired to be studied or sequenced, etc., and may include one, or more than one, cell type. The cells may be for example, from a specific population of cells, such as from a certain organ or tissue (e.g., cardiac cells, immune cells, muscle cells, cancer cells, etc.), cells from a specific individual or species (e.g., human cells, mouse cells, bacteria, etc.), cells from different organisms, cells from a naturally-occurring sample (e.g., pond water, soil, etc.), or the like. In some cases, the cells may be dissociated from tissue. In embodiments, the method does not include dissociating the cell from the tissue or the cellular microenvironment. In embodiments, the method does not include lysing the cell.

In embodiments, the method further includes subjecting the cell to expansion microscopy methods and techniques. Expansion allows individual targets (e.g., mRNA or RNA transcripts) which are densely packed within a cell, to be resolved spatially in a high-throughput manner. Expansion microscopy techniques are known in the art and can be performed as described in US 2016/0116384 and Chen et al., Science, 347, 543 (2015), each of which are incorporated herein by reference in their entirety.

In embodiments, the method does not include subjecting the cell to expansion microscopy. Typically, expansion microscopy techniques utilize a swellable polymer or hydrogel (e.g., a synthetic matrix-forming material) which can significantly slow diffusion of enzymes and nucleotides. Matrix (e.g., synthetic matrix) forming materials include polyacrylamide, cellulose, alginate, polyamide, cross-linked agarose, cross-linked dextran or cross-linked polyethylene glycol. The matrix forming materials can form a matrix by polymerization and/or crosslinking of the matrix forming materials using methods specific for the matrix forming materials and methods, reagents and conditions known to those of skill in the art. Additionally, expansion microscopy techniques may render the temperature of the cell sample difficult to modulate in a uniform, controlled manner. Modulating temperature provides a useful parameter to optimize amplification and sequencing methods. In embodiments, the method does not include an exogenous matrix.

In embodiments, the first oligonucleotide, second oligonucleotide, or both the first oligonucleotide and second oligonucleotide contain one or more functional moieties (e.g., bioconjugate reactive groups) that serve as attachment points to the cell (i.e., the internal cellular scaffold) or to the matrix in which the cell is embedded (e.g. a hydrogel). In embodiments, the first oligonucleotide probe, second oligonucleotide probe, or both the first oligonucleotide probe and second oligonucleotide probe contain one or more functional moieties (e.g., bioconjugate reactive groups) that serve as attachment points to the cell (i.e., the internal cellular scaffold) or to the matrix in which the cell is embedded (e.g. a hydrogel). In embodiments, the bioconjugate reactive group is located at the 5′ and/or 3′ end of the oligonucleotide. In embodiments, the bioconjugate reactive group is located at an internal position of the oligonucleotide e.g., the oligonucleotide contains one or more modified nucleotides, such as aminoallyl deoxyuridine 5′-triphosphate (dUTP) nucleotide(s). In embodiments, the functional moiety can be covalently cross-linked, copolymerize with or otherwise non-covalently bound to the matrix. In embodiments, the functional moiety can react with a cross-linker. In embodiments, the functional moiety can be part of a ligand-ligand binding pair. Suitable exemplary functional moieties include an amine, acrydite, alkyne, biotin, azide, and thiol. In embodiments of crosslinking, the functional moiety is cross-linked to modified dNTP or dUTP or both. In embodiments, suitable exemplary cross-linker reactive groups include imidoester (DMP), succinimide ester (NHS), maleimide (Sulfo-SMCC), carbodiimide (DCC, EDC) and phenyl azide. Cross-linkers within the scope of the present disclosure may include a spacer moiety. In embodiments, such spacer moieties may be functionalized. In embodiments, such spacer moieties may be chemically stable. In embodiments, such spacer moieties may be of sufficient length to allow amplification of the nucleic acid bound to the matrix. In embodiments, suitable exemplary spacer moieties include polyethylene glycol, carbon spacers, photo-cleavable spacers and other spacers known to those of skill in the art and the like. In embodiments, the oligonucleotide primer contains a modified nucleotide (e.g., amino-allyl dUTP, 5-TCO-PEG4-dUTP, C8-Alkyne-dUTP, 5-Azidomethyl-dUTP, 5-Vinyl-dUTP, or 5-Ethynyl dLTTP). For example, prior to amplification, the modified nucleotide-containing primer is attached to the cell protein matrix by using a cross-linking reagent (e.g., an amine-reactive crosslinking agent with PEG spacers, such as (PEGylated bis(sulfosuccinimidyl)suberate) (BS(PEG)₉)).

In embodiments, the target polynucleotide includes DNA nucleic acid sequences. In embodiments, the target polynucleotide includes an endogenous sequence (e.g., genomic nucleic acid sequences present within the cell). In embodiments, the target polynucleotide is a cDNA target polynucleotide and before step a), an RNA nucleic acid sequence is reverse transcribed to generate the cDNA target polynucleotide.

In embodiments, ligating includes enzymatic ligation including a ligation enzyme (e.g., Circligase enzyme, Taq DNA Ligase, HiFi Taq DNA Ligase, T4 ligase, PBCV-1 DNA Ligase (also known as SplintR ligase) or Ampligase DNA Ligase). Non-limiting examples of ligases include DNA ligases such as DNA Ligase I, DNA Ligase II, DNA Ligase III, DNA Ligase IV, T4 DNA ligase, T7 DNA ligase, T3 DNA Ligase, E. coli DNA Ligase, PBCV-1 DNA Ligase (also known as SplintR ligase) or a Taq DNA Ligase. In embodiments, the ligase enzyme includes a T4 DNA ligase, T4 RNA ligase 1, T4 RNA ligase 2, T3 DNA ligase or T7 DNA ligase. In embodiments, the enzymatic ligation is performed by a mixture of ligases. In embodiments, the ligation enzyme is selected from the group consisting of T4 DNA ligase, T4 RNA ligase 1, T4 RNA ligase 2, RtcB ligase, T3 DNA ligase, T7 DNA ligase, Taq DNA ligase, PBCV-1 DNA Ligase, a thermostable DNA ligase (e.g., 5′AppDNA/RNA ligase), an ATP dependent DNA ligase, an RNA-dependent DNA ligase (e.g., SplintR ligase), and combinations thereof. In embodiments, enzymatic ligation includes two different ligation enzymes (e.g., SplintR ligation and T4 DNA ligase, or SplintR ligase and Taq DNA ligase). For example, one ligase enzyme may show improved ligation efficiency for ligating the complementary sequence of the extended second oligonucleotide primer to the first oligonucleotide primer in the gap-filled region, while a second ligase enzyme has improved ligation efficiency for ligating the 3′ end of the first oligonucleotide primer to the 5′ end of the second oligonucleotide primer. In embodiments, enzymatic ligation includes more than two different ligation enzymes.

In embodiments, the oligonucleotide probe includes a blocking moiety at the 3′ end (e.g., at the 3′ end of the target oligonucleotide probe, the first oligonucleotide, the second oligonucleotide, the first oligonucleotide probe, or the second oligonucleotide probe). In embodiments, a terminal nucleotide of the oligonucleotide probe includes a blocking moiety. In embodiments, the blocking moiety is reversible. In embodiments, the blocking moiety is irreversible. In embodiments, the blocking moiety at the 3′ end (e.g., the 3′ blocking moiety) includes a reversible terminator. In embodiments, the 3′ blocking moiety includes a dideoxynucleotide triphosphate (e.g., a ddNTP).

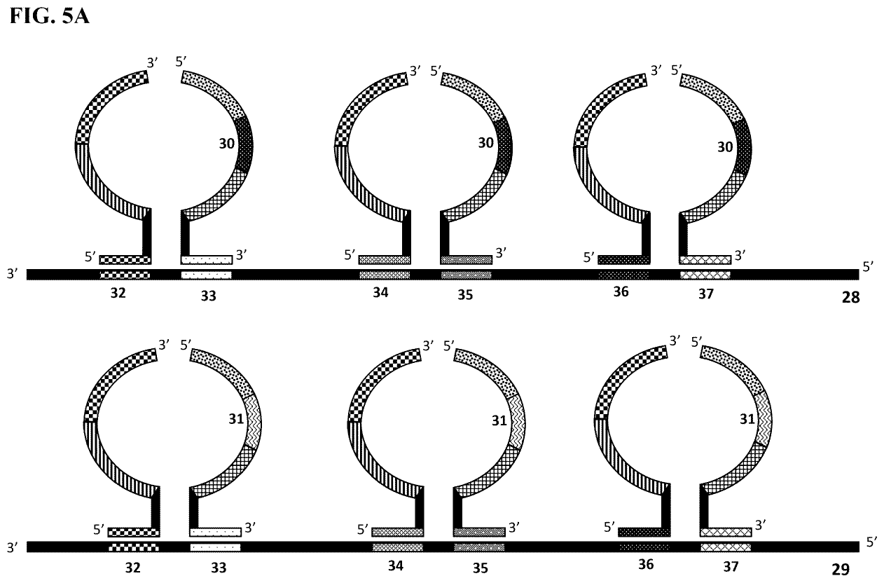

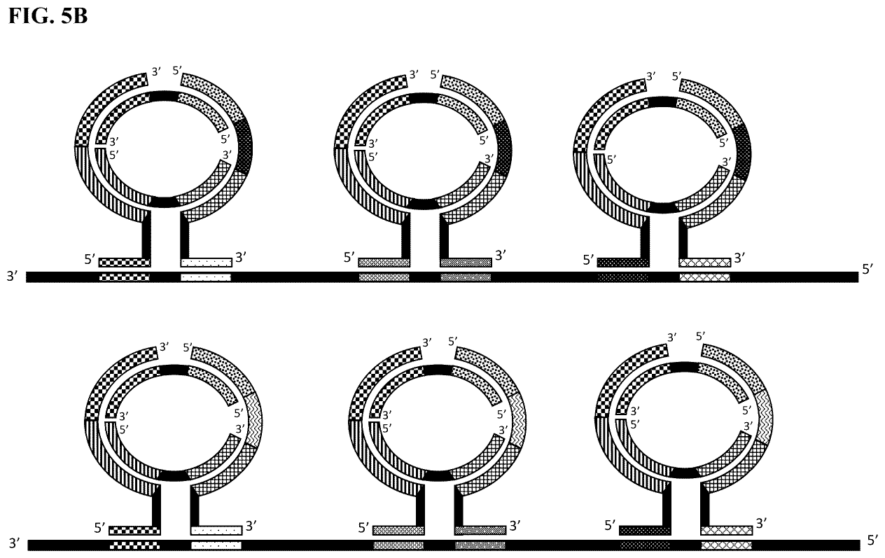

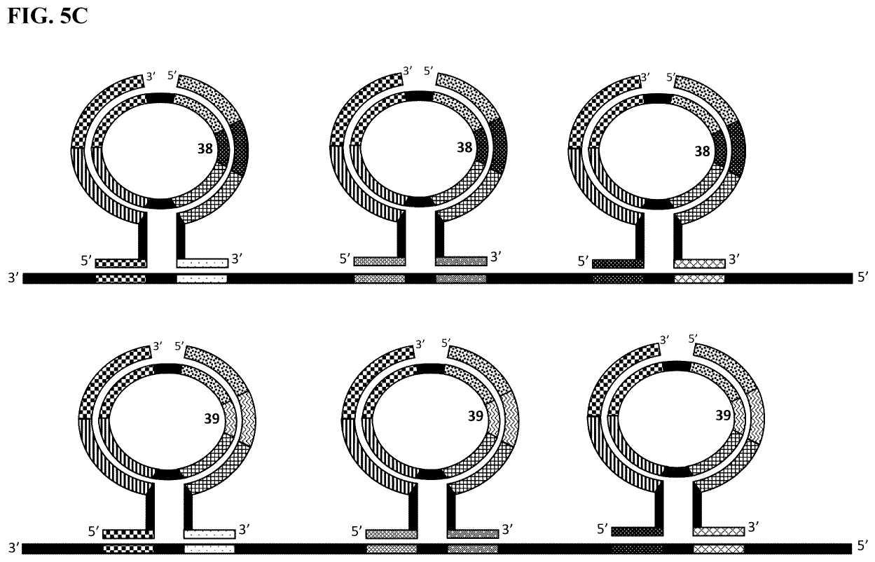

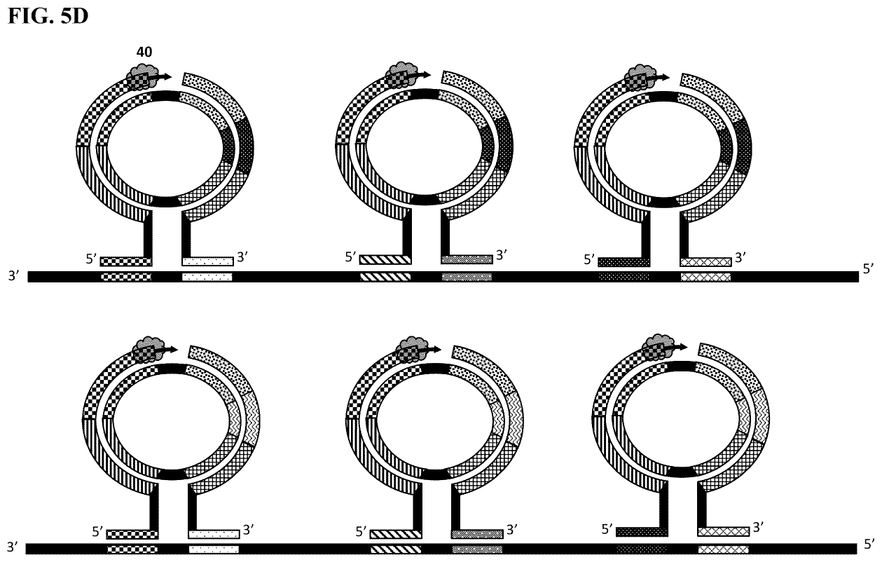

One having skill in the art would understand that the gap fill (e.g., gap filling between the 5′ end of the first oligonucleotide probe and the 3′ end of the second oligonucleotide probe, when hybridized to a first oligonucleotide including a barcode sequence) and ligation (e.g., ligation of the 3′ end of the first oligonucleotide probe and the 5′ end of the second oligonucleotide probe, when hybridized to complementary sequences in the first and second oligonucleotides) steps may occur simultaneously (e.g., as illustrated in FIG. 5C) or in separate and distinct steps. For example, in embodiments, the gap between the two hybridization sequences of the third and second oligonucleotide probes (e.g., the gap include the barcode sequence of the first oligonucleotide) is polymerized (e.g., with a DNA polymerase), then the second oligonucleotide is hybridized to the complementary sequences of the third and second oligonucleotide probes (e.g., the 5′ end of the second oligonucleotide probe and the 3′ end of the first oligonucleotide probe) and subsequently ligated to close and generate a circularized oligonucleotide. In other embodiments, the second oligonucleotide is initially hybridized to the third and second oligonucleotide probes, then the gap between the 3′ end of the second oligonucleotide probe and the 5′ end of the first oligonucleotide probe is polymerized, and subsequently ligation is performed to join the two oligonucleotide probes and generate a circularized oligonucleotide. In yet other embodiments, the second oligonucleotide is initially hybridized to the third and second oligonucleotide probes, then the between the 3′ end of the second oligonucleotide probe and the 5′ end of the first oligonucleotide probe is polymerized, while simultaneously ligation is performed to join the third and second oligonucleotide probes and generate a circularized oligonucleotide. In other embodiments, the second oligonucleotide is hybridized to the third and second oligonucleotide probes while the gap between the 3′ end of the second oligonucleotide probe and the 5′ end of the first oligonucleotide probe is polymerized, and subsequently ligation is performed to join the third and second oligonucleotide probes and generate a circularized oligonucleotide. In yet other embodiments, the second oligonucleotide is hybridized to the third and second oligonucleotide probes while the gap between the 3′ end of the second oligonucleotide probe and the 5′ end of the first oligonucleotide probe is polymerized, while simultaneously ligation is performed to join the third and second oligonucleotide probes and generate a circularized oligonucleotide.

In embodiments, ligating includes chemical ligation (e.g., enzyme-free, click-mediated ligation). In embodiments, the oligonucleotide probes include a first bioconjugate reactive moiety capable of bonding upon contact with a second (complementary) bioconjugate reactive moiety. In embodiments, the oligonucleotide probes includes an alkynyl moiety at the 3′ and an azide moiety at the 5′ end that, upon hybridization to the target nucleic acid react to form a triazole linkage during suitable reaction conditions. Reaction conditions and protocols for chemical ligation techniques that are compatible with nucleic acid amplification methods are known in the art, for example El-Sagheer, A. H., & Brown, T. (2012). Accounts of chemical research, 45(8), 1258-1267; Manuguerra I. et al. Chem Commun (Camb). 2018; 54(36):4529-4532; and Odch, F., et al. (2019). Molecules (Basel, Switzerland), 25(1), 3, each of which is incorporated herein by reference in their entirety.

In embodiments, the method further includes an amplification method for amplifying the circular oligonucleotide. In embodiments, the method further includes amplifying the circular oligonucleotide by extending an amplification primer with a polymerase (e.g., a strand-displacing polymerase), wherein the primer extension generates an extension product including multiple complements of the circular oligonucleotide, referred to as an amplicon. An amplicon typically contains multiple, tandem copies of the circularized nucleic acid molecule of the corresponding sample nucleic acid. The number of copies can be varied by appropriate modification of the reaction conditions, such as varying the number of amplification cycles, using polymerases of varying processivity in the amplification reaction, or varying the length of time that the amplification reaction is run. In embodiments, the extension product includes three or more copies of the circular oligonucleotide. In embodiments, the circular oligonucleotide is copied about 3-50 times (i.e., the extension product includes about 3 to 50 complements of the circular oligonucleotide). In embodiments, the circular oligonucleotide is copied about 50-100 times (i.e., the extension product includes about 50 to 100 complements of the circular oligonucleotide). In embodiments, the circular oligonucleotide is copied about 100-300 times (i.e., the extension product includes about 100 to 300 complements of the circular oligonucleotide). In embodiments, the method includes hybridizing an amplification primer and oligonucleotide primer in the same reaction (e.g., simultaneously). In embodiments, the oligonucleotide probe is extended as an amplification primer after generating the circular oligonucleotide (e.g., the 3′ end of the oligonucleotide probe hybridized to the circular oligonucleotide is extended with a polymerase). In embodiments, the method includes contacting the target with an amplification primer and oligonucleotide primer in the same reaction (e.g., simultaneously).

In embodiments, the amplifying includes rolling circle amplification (RCA) or rolling circle transcription (RCT) (see, e.g., Lizardi et al., Nat. Genet. 19:225-232 (1998), which is incorporated herein by reference in its entirety). Several suitable rolling circle amplification methods are known in the art. For example, RCA amplifies a circular polynucleotide (e.g., DNA) by polymerase extension of an amplification primer complementary to a portion of the template polynucleotide. This process generates copies of the circular polynucleotide template such that multiple complements of the template sequence arranged end to end in tandem are generated (i.e., a concatemer) locally preserved at the site of the circle formation. In embodiments, the amplifying occurs at isothermal conditions. In embodiments, the amplifying includes hybridization chain reaction (HCR). HCR uses a pair of complementary, kinetically trapped hairpin oligomers to propagate a chain reaction of hybridization events, as described in Dirks, R. M., & Pierce, N. A. (2004) PNAS USA, 101(43), 15275-15278, which is incorporated herein by reference for all purposes. In embodiments, the amplifying includes branched rolling circle amplification (BRCA); e.g., as described in Fan T, Mao Y, Sun Q, et al. Cancer Sci. 2018; 109:2897-2906, which is incorporated herein by reference in its entirety. In embodiments, the amplifying includes hyberbranched rolling circle amplification (HRCA). Hyperbranched RCA uses a second primer complementary to the first amplification product. This allows products to be replicated by a strand-displacement mechanism, which yields drastic amplification within an isothermal reaction (Lage et al., Genome Research 13:294-307 (2003), which is incorporated herein by reference in its entirety). In embodiments, amplifying includes polymerase extension of an amplification primer. In embodiments, the polymerase is T4, T7, Sequenase, Taq, Klenow, and Pol I DNA polymerases. SD polymerase, Bst large fragment polymerase, or a phi29 polymerase or mutant thereof. In embodiments, the strand-displacing enzyme is an SD polymerase, Bst large fragment polymerase, or a phi29 polymerase or mutant thereof. In embodiments, the strand-displacing polymerase is phi29 polymerase, phi29 mutant polymerase or a thermostable phi29 mutant polymerase. A “phi polymerase” (or “Φ29 polymerase”) is a DNA polymerase from the @29 phage or from one of the related phages that, like Φ29, contain a terminal protein used in the initiation of DNA replication. For example, phi29 polymerases include the B103, GA-1, PZA, Φ15, BS32, M2Y (also known as M2), Nf, G1, Cp-1, PRD1, PZE, SFS, Cp-5, Cp-7, PR4, PR5, PR722, L17, Φ21, and AV-1 DNA polymerases, as well as chimeras thereof. A phi29 mutant DNA polymerase includes one or more mutations relative to naturally-occurring wild-type phi29 DNA polymerases, for example, one or more mutations that alter interaction with and/or incorporation of nucleotide analogs, increase stability, increase read length, enhance accuracy, increase phototolerance, and/or alter another polymerase property, and can include additional alterations or modifications over the wild-type phi29 DNA polymerase, such as one or more deletions, insertions, and/or fusions of additional peptide or protein sequences. Thermostable phi29 mutant polymerases are known in the art, see for example US 2014/0322759, which is incorporated herein by reference for all purposes. For example, a thermostable phi29 mutant polymerase refers to an isolated bacteriophage phi29 DNA polymerase including at least one mutation selected from the group consisting of M8R, V51A, M97T, L123S, G197D, K209E, E221K, E239G, Q497P, K512E, E515A, and F526 (relative to wild type phi29 polymerase). In embodiments, the polymerase is a phage or bacterial RNA polymerases (RNAPs). In embodiments, the polymerase is a T7 RNA polymerase. In embodiments, the polymerase is an RNA polymerase. Useful RNA polymerases include, but are not limited to, viral RNA polymerases such as T7 RNA polymerase, T3 polymerase, SP6 polymerase, and Kll polymerase; Eukaryotic RNA polymerases such as RNA polymerase I, RNA polymerase II, RNA polymerase III, RNA polymerase IV, and RNA polymerase V; and Archaca RNA polymerase.

In embodiments, the amplification method includes a standard dNTP mixture including dATP, dCTP, dGTP and dTTP (for DNA) or dATP, dCTP, dGTP and dUTP (for RNA). In embodiments, the amplification method includes a mixture of standard dNTPs and modified nucleotides that contain functional moieties (e.g., bioconjugate reactive groups) that serve as attachment points to the cell or the matrix in which the cell is embedded (e.g. a hydrogel). In embodiments, the amplification method includes a mixture of standard dNTPs and modified nucleotides that contain functional moieties (e.g., bioconjugate reactive groups) that participate in the formation of a bioconjugate linker. The modified nucleotides may react and link the amplification product to the surrounding cell scaffold. For example, amplifying may include an extension reaction wherein the polymerase incorporates a modified nucleotide into the amplification product, wherein the modified nucleotide includes a bioconjugate reactive moiety (e.g., an alkynyl moiety) attached to the nucleobase. The bioconjugate reactive moiety of the modified nucleotide participates in the formation of a bioconjugate linker by reacting with a complementary bioconjugate reactive moiety present in the cell (e.g., a crosslinking agent, such as NHS-PEG-azide, or an amine moiety) thereby attaching the amplification product to the internal scaffold of the cell. In embodiments, the functional moiety can be covalently cross-linked, copolymerize with or otherwise non-covalently bound to the matrix. In embodiments, the functional moiety can react with a cross-linker. In embodiments, the functional moiety can be part of a ligand-ligand binding pair. Suitable exemplary functional moieties include an amine, acrydite, alkyne, biotin, azide, and thiol. In embodiments of crosslinking, the functional moiety is cross-linked to modified dNTP or dUTP or both. In embodiments, suitable exemplary cross-linker reactive groups include imidoester (DMP), succinimide ester (NHS), maleimide (Sulfo-SMCC), carbodiimide (DCC, EDC) and phenyl azide. Cross-linkers within the scope of the present disclosure may include a spacer moiety. In embodiments, such spacer moieties may be functionalized. In embodiments, such spacer moieties may be chemically stable. In embodiments, such spacer moieties may be of sufficient length to allow amplification of the nucleic acid bound to the matrix. In embodiments, suitable exemplary spacer moieties include polyethylene glycol, carbon spacers, photo-cleavable spacers and other spacers known to those of skill in the art and the like. In embodiments, amplification reactions include standard dNTPs and a modified nucleotide (e.g., amino-allyl dUTP, 5-TCO-PEG4-dUTP, C8-Alkyne-dUTP, 5-Azidomethyl-dUTP, 5-Vinyl-dUTP, or 5-Ethynyl dLTTP). For example, during amplification a mixture of standard dNTPs and aminoallyl deoxyuridine 5′-triphosphate (dUTP) nucleotides may be incorporated into the amplicon and subsequently cross-linked to the cell protein matrix by using a cross-linking reagent (e.g., an amine-reactive crosslinking agent with PEG spacers, such as (PEGylated bis(sulfosuccinimidyl)suberate) (BS(PEG)9)).

In embodiments, the amplification primer and the sequencing primer includes an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers (e.g., amplification primer or sequencing primer) include nucleotides ranging from 17 to 30 nucleotides. In embodiments, the primer is at least 17 nucleotides, or alternatively, at least 18 nucleotides, or alternatively, at least 19 nucleotides, or alternatively, at least 20 nucleotides, or alternatively, at least 21 nucleotides, or alternatively, at least 22 nucleotides, or alternatively, at least 23 nucleotides, or alternatively, at least 24 nucleotides, or alternatively, at least 25 nucleotides, or alternatively, at least 26 nucleotides, or alternatively, at least 27 nucleotides, or alternatively, at least 28 nucleotides, or alternatively, at least 29 nucleotides, or alternatively, at least 30 nucleotides, or alternatively at least 50 nucleotides, or alternatively at least 75 nucleotides or alternatively at least 100 nucleotides. In embodiments, one or more nucleotides within the amplification primer sequence, the sequencing primer sequence, and/or the immobilized first and/or second oligonucleotide primer contains one or more functional moieties (e.g., bioconjugate reactive groups) that serve as attachment points to the cell (e.g., to an internal cellular component, such as a protein) or the matrix in which the cell is embedded (e.g. a hydrogel). In embodiments, one or more nucleotides within the amplification primer sequence, the sequencing primer sequence, and/or the first and/or second immobilized oligonucleotide primer contains one or more functional moieties (e.g., bioconjugate reactive groups) that serve as attachment points to complementary bioconjugate reactive groups within the cell (e.g., a protein). In embodiments, a plurality of oligonucleotide primers are provided to the matrix in which the cell is embedded prior to amplification. In embodiments, a plurality of oligonucleotide primers are provided to the matrix in which the cell is embedded concurrently with amplification. In embodiments, the bioconjugate reactive group is located at the 5′ or 3′ end of the primer. In embodiments, the bioconjugate reactive group is located at an internal position of the primer e.g., the primer contains one or more modified nucleotides, such as aminoallyl deoxyuridine 5′-triphosphate (dUTP) nucleotide(s). In embodiments, the functional moiety can be covalently cross-linked, copolymerize with or otherwise non-covalently bound to the matrix. In embodiments, the functional moiety can react with a cross-linker. In embodiments, the functional moiety can be part of a ligand-ligand binding pair. Suitable exemplary functional moieties include an amine, acrydite, alkyne, biotin, azide, and thiol. In embodiments of crosslinking, the functional moiety is cross-linked to modified dNTP or dUTP or both. In embodiments, suitable exemplary cross-linker reactive groups include imidoester (DMP), succinimide ester (NHS), maleimide (Sulfo-SMCC), carbodiimide (DCC, EDC) and phenyl azide. Cross-linkers within the scope of the present disclosure may include a spacer moiety. In embodiments, such spacer moieties may be functionalized. In embodiments, such spacer moieties may be chemically stable. In embodiments, such spacer moieties may be of sufficient length to allow amplification of the nucleic acid bound to the matrix. In embodiments, suitable exemplary spacer moieties include polyethylene glycol, carbon spacers, photo-cleavable spacers and other spacers known to those of skill in the art and the like. In embodiments, the amplification primer and/or the sequencing primer contains a modified nucleotide (e.g., amino-allyl dUTP, 5-TCO-PEG4-dUTP, C8-Alkyne-dUTP, 5-Azidomethyl-dUTP, 5-Vinyl-dUTP, or 5-Ethynyl dLTTP). For example, prior to amplification, the modified nucleotide-containing primer is attached to the cell protein matrix by using a cross-linking reagent (e.g., an amine-reactive crosslinking agent with PEG spacers, such as (PEGylated bis(sulfosuccinimidyl)suberate) (BS(PEG)9)).

In embodiments, the method includes amplifying the circular polynucleotide by extending an amplification primer with a strand-displacing polymerase, wherein the primer extension generates an extension product including multiple complements of the circular polynucleotide. In embodiments, the method of amplifying includes an isothermal amplification method. In embodiments, the method of amplifying includes rolling circle amplification (RCA) or rolling circle transcription (RCT). In embodiments, the method of amplifying is rolling circle amplification (RCA). In embodiments, amplifying includes exponential rolling circle amplification (eRCA). Exponential RCA is similar to the linear process except that it uses a second primer (e.g., one or more immobilized oligonucleotide(s)) having a sequence that is identical to at least a portion of the circular template (Lizardi et al. Nat. Genet. 19:225 (1998)). This two-primer system achieves isothermal, exponential amplification. Exponential RCA has been applied to the amplification of non-circular DNA through the use of a linear probe that binds at both of its ends to contiguous regions of a target DNA followed by circularization using DNA ligase (Nilsson et al. Science 265(5181):208 5(1994)).

In embodiments, amplifying the circular oligonucleotide includes incubation with a strand-displacing polymerase. In embodiments, amplifying includes incubation with a strand-displacing polymerase for about 10 seconds to about 60 minutes. In embodiments, amplifying includes incubation with a strand-displacing polymerase for about 60 seconds to about 60 minutes. In embodiments, amplifying includes incubation with a strand-displacing polymerase for about 10 minutes to about 60 minutes. In embodiments, amplifying includes incubation with a strand-displacing polymerase for about 10 minutes to about 30 minutes. In embodiments, amplifying includes incubation with a strand-displacing polymerase at a temperature of about 20° C. to about 50° C. In embodiments, incubation with the strand-displacing polymerase is at a temperature of about 35° ° C. to 42° C. In embodiments, the strand-displacing polymerase is phi29 polymerase, SD polymerase, Bst large fragment polymerase, phi29 mutant polymerase, or a thermostable phi29 mutant polymerase.

In embodiments, the method includes forming the template polynucleotide. The template polynucleotide can be a circular, dumbbell-shaped, or other closed nucleic acid molecule configuration that does not have a free 3′ or 5′ end. Typical library preparation steps may be performed on a linear template such that it is circularized (e.g., such as the protocols described in Kershaw, C. J., & O'Keefe, R. T. (2012) 941, 257-269). The initial template polynucleotide molecules can vary length, such as about 100-300 nucleotides long, about 300-500 nucleotides long, or about 500-1000 nucleotides long. In embodiments, the initial template polynucleotide molecular is about 100-1000 nucleotides, about 150-950 nucleotides, about 200-900 nucleotides, about 250-850 nucleotides, about 300-800 nucleotides, about 350-750 nucleotides, about 400-700 nucleotides, or about 450-650 nucleotides. In embodiments, the initial template polynucleotide molecule is about 150 nucleotides. In embodiments, the initial template polynucleotide is about 100-1000 nucleotides long. In embodiments, the initial template polynucleotide is about 100-300 nucleotides long. In embodiments, the initial template polynucleotide is about 300-500 nucleotides long. In embodiments, the initial template polynucleotide is about 500-1000 nucleotides long. In embodiments, the initial template polynucleotide molecule is about 100 nucleotides. In embodiments, the initial template polynucleotide molecule is about 300 nucleotides. In embodiments, the initial template polynucleotide molecule is about 500 nucleotides. In embodiments, the initial template polynucleotide molecule is about 1000 nucleotides.

In embodiments, the amplification primer is attached to the solid surface. In embodiments, the amplification primer is in solution. In embodiments, the amplification primer includes one or more phosphorothioate nucleotides. In embodiments, the amplification primer includes a plurality of phosphorothioate nucleotides. In embodiments, about or at least about 20%, 30%, 40%, 50%, 60%, 70%, 80%, 90%, or about 100% of the nucleotides in the amplification primer are phosphorothioate nucleotides. In embodiments, most of the nucleotides in the amplification primer are phosphorothioate nucleotides. In embodiments, all of the nucleotides in the amplification primer are phosphorothioate nucleotides.

Amplification primer molecules can be fixed to surface by a variety of techniques, including covalent attachment and non-covalent attachment. In embodiments, the amplification primers are confined to an area of a discrete region (referred to as a cluster). The discrete regions may have defined locations in a regular array, which may correspond to a rectilinear pattern, circular pattern, hexagonal pattern, or the like. A regular array of such regions is advantageous for detection and data analysis of signals collected from the arrays during an analysis. These discrete regions are separated by interstitial regions. As used herein, the term “interstitial region” refers to an area in a substrate or on a surface that separates other areas of the substrate or surface. For example, an interstitial region can separate one concave feature of an array from another concave feature of the array. The two regions that are separated from each other can be discrete, lacking contact with each other. In another example, an interstitial region can separate a first portion of a feature from a second portion of a feature. In embodiments the interstitial region is continuous whereas the features are discrete, for example, as is the case for an array of wells in an otherwise continuous surface. The separation provided by an interstitial region can be partial or full separation. Interstitial regions will typically have a surface material that differs from the surface material of the features on the surface. For example, features of an array can have primers that exceeds the amount or concentration present at the interstitial regions. In some embodiments the primers may not be present at the interstitial regions. In embodiments, the amplification primer is attached to a solid support and a template polynucleotide is hybridized to the primer. In embodiments, at least two different primers are attached to the solid support (e.g., a forward and a reverse primer), which facilitates generating multiple amplification products from the first extension product or a complement thereof.

In embodiments, extending the amplification primer includes incubation with the strand-displacing polymerase in suitable conditions and for a suitable amount of time. In embodiments, the step of extending the amplification primer includes incubation with the strand-displacing polymerase (i) for about 10 seconds to about 30 minutes, and/or (ii) at a temperature of about 20° ° C. to about 50° C. In embodiments, incubation with the strand-displacing polymerase is for about 0.5 minutes to about 16 minutes. In embodiments, incubation with the strand-displacing polymerase is for about 0.5 minutes to about 10 minutes. In embodiments, incubation with the strand-displacing polymerase is for about 1 minutes to about 5 minutes. In embodiments, the method includes amplifying a template polynucleotide by extending an amplification primer with a strand-displacing polymerase for about 10 seconds to about 30 minutes. In embodiments, the method includes amplifying a template polynucleotide by extending an amplification primer with a strand-displacing polymerase for about 30 seconds to about 16 minutes. In embodiments, the method includes amplifying a template polynucleotide by extending an amplification primer with a strand-displacing polymerase for about 30 seconds to about 10 minutes. In embodiments, the method includes amplifying a template polynucleotide by extending an amplification primer with a strand-displacing polymerase for about 30 seconds to about 5 minutes. In embodiments, the method includes amplifying a template polynucleotide by extending an amplification primer with a strand-displacing polymerase for about 1 second to about 5 minutes. In embodiments, the method includes amplifying a template polynucleotide by extending an amplification primer with a strand-displacing polymerase for about 1 second to about 2 minutes.

In embodiments, incubation with the strand-displacing polymerase is at a temperature of about 35° C. to 42° C. In embodiments, incubation with the strand-displacing polymerase is at a temperature of about 37° C. to 40° C. In embodiments, incubation with the thermostable strand-displacing polymerase is at a temperature of about 40° C. to 80° C. In embodiments, the method includes amplifying a template polynucleotide by extending an amplification primer with a strand-displacing polymerase at a temperature of about 20° C. to about 50° C. In embodiments, the method includes amplifying a template polynucleotide by extending an amplification primer with a strand-displacing polymerase at a temperature of about 30° ° C. to about 50° C. In embodiments, the method includes amplifying a template polynucleotide by extending an amplification primer with a strand-displacing polymerase at a temperature of about 25° C. to about 45° C. In embodiments, the method includes amplifying a template polynucleotide by extending an amplification primer with a strand-displacing polymerase at a temperature of about 35° C. to about 45° C. In embodiments, the method includes amplifying a template polynucleotide by extending an amplification primer with a strand-displacing polymerase at a temperature of about 35° ° C. to about 42° ° C. In embodiments, the method includes amplifying a template polynucleotide by extending an amplification primer with a strand-displacing polymerase at a temperature of about 37° C. to about 40° C.

In embodiments, the extension product includes three or more copies of the target nucleic acid (e.g., the barcode sequence). In embodiments, the extension product includes at least three or more copies of the target nucleic acid. In embodiments, the extension product includes at least five or more copies of the target nucleic acid. In embodiments, the extension product includes at 5 to 10 copies of the target nucleic acid. In embodiments, the extension product includes 10 to 20 copies of the target nucleic acid. In embodiments, the extension product includes 20 to 50 copies of the target nucleic acid.

In embodiments, the first oligonucleotide, second oligonucleotide, first oligonucleotide probe, and/or second oligonucleotide probe is covalently attached to the matrix or to a cellular component via a bioconjugate reactive linker. In embodiments, the 5′ end of the oligonucleotide contains a functional group that is capable of reacting with a complementary group so the primer may be tethered to a cellular component (e.g., a protein). In embodiments, the 3′ end of the oligonucleotide contains a functional group that is capable of reacting with a complementary group so the primer may be tethered to a cellular component (e.g., a protein). Non-limiting examples of covalent attachment include amine-modified polynucleotides within the oligonucleotide reacting with epoxy or isothiocyanate groups within the matrix, succinylated polynucleotides within the oligonucleotide reacting with aminophenyl or aminopropyl functional groups within the matrix, dibenzocycloctyne-modified polynucleotides within the oligonucleotide reacting with azide functional groups within the matrix (or vice versa), trans-cyclooctyne-modified polynucleotides within the oligonucleotide reacting with tetrazine or methyl tetrazine groups within the matrix (or vice versa), disulfide modified polynucleotides within the oligonucleotide reacting with mercapto-functional groups within the matrix, amine-functionalized polynucleotides within the oligonucleotide reacting with carboxylic acid groups within the matrix or cellular component via 1-ethyl-3-(3-dimethylaminopropyl)-carbodiimide hydrochloride (EDC) chemistry, thiol-modified polynucleotides within the oligonucleotide attaching to the matrix or cellular component via a disulfide bond or maleimide linkage, alkyne-modified polynucleotides within the oligonucleotide attaching to a matrix via copper-catalyzed click reactions to azide functional groups within the matrix, azide-modified polynucleotides within the oligonucleotide attaching to the matrix via copper-catalyzed click reactions to alkyne functional groups within the matrix, and acrydite-modified polynucleotides within the oligonucleotide polymerizing with free acrylic acid monomers within the matrix to form polyacrylamide. In embodiments, the oligonucleotide is attached to the matrix through electrostatic binding. For example, the negatively charged phosphate backbone of the oligonucleotide may be bound electrostatically to positively charged monomers in the solid support.

In embodiments, the oligonucleotide includes a first bioconjugate reactive group. In embodiments, the oligonucleotide is attached to a cellular compartment. In embodiments, the cellular component includes a second bioconjugate reactive group. In embodiments, the first bioconjugate reactive group is attached to the second bioconjugate reactive group by covalent or non-covalent bonding. In embodiments, the oligonucleotide is covalently attached to a cellular component. In embodiments, the 5′ end of the oligonucleotide contains a functional group that is tethered to the cellular component. In embodiments, the oligonucleotide is covalently attached to a matrix within the cell. In embodiments, the 5′ end of the oligonucleotide contains a functional group that is tethered to the matrix within the cell. Non-limiting examples of covalent attachment include amine-modified polynucleotides reacting with epoxy or isothiocyanate groups in the cell or matrix within the cell, succinylated polynucleotides reacting with aminophenyl or aminopropyl functional groups in the cell or matrix within the cell, dibenzocycloctyne-modified polynucleotides reacting with azide functional groups in the cell or matrix within the cell (or vice versa), trans-cyclooctyne-modified polynucleotides reacting with tetrazine or methyl tetrazine groups in the cell or matrix within the cell (or vice versa), disulfide modified polynucleotides reacting with mercapto-functional groups in the cell or matrix within the cell, amine-functionalized polynucleotides reacting with carboxylic acid groups in the cell or matrix within the cell via 1-ethyl-3-(3-dimethylaminopropyl)-carbodiimide hydrochloride (EDC) chemistry, thiol-modified polynucleotides attaching to cell or matrix within the cell via a disulphide bond or maleimide linkage, alkyne-modified polynucleotides attaching to the cell or matrix within the cell via copper-catalyzed click reactions to azide functional groups in the cell or matrix within the cell, and acrydite-modified polynucleotides polymerizing with free acrylic acid monomers in the cell or matrix within the cell to form polyacrylamide or reacting with thiol groups in the cell or matrix within the cell. In embodiments, the oligonucleotide is attached to the polymer through electrostatic binding. For example, the negatively charged phosphate backbone of the oligonucleotide may be bound electrostatically to positively charged monomers in the matrix.

In embodiments, the first oligonucleotide, second oligonucleotide, first oligonucleotide probe, and/or second oligonucleotide probe is attached to the matrix or to a cellular component via a specific binding reagent. In embodiments, the specific binding reagent includes an antibody, single-chain Fv fragment (scFv), antibody fragment-antigen binding (Fab), or an aptamer. In embodiments, the specific binding reagent includes an antibody, or antigen binding fragment, an aptamer, affimer, or non-immunoglobulin scaffold. In embodiments, the specific binding reagent is a peptide, a cell penetrating peptide, an aptamer, a DNA aptamer, an RNA aptamer, an antibody, an antibody fragment, a light chain antibody fragment, a single-chain variable fragment (scFv), a lipid, a lipid derivative, a phospholipid, a fatty acid, a triglyceride, a glycerolipid, a glycerophospholipid, a sphingolipid, a saccharolipid, a polyketide, a polylysine, polyethyleneimine, diethylaminocthyl (DEAE)-dextran, cholesterol, or a sterol moiety. For example, the matrix or cellular component (e.g., a protein) may contain a complementary specific binding reagent to the oligonucleotide containing a specific binding reagent.

In embodiments, the method further includes detecting the amplification product (e.g., the amplification product of step (f)). In embodiments, detecting includes two-dimensional (2D) or three-dimensional (3D) fluorescent microscopy. Suitable imaging technologies are known in the art, as exemplified by Larsson et al., Nat. Methods (2010) 7:395-397 and associated supplemental materials, the entire content of which is incorporated by reference herein in its entirety. In embodiments of the methods provided herein, the imaging is accomplished by confocal microscopy. Confocal fluorescence microscopy involves scanning a focused laser beam across the sample, and imaging the emission from the focal point through an appropriately-sized pinhole. This suppresses the unwanted fluorescence from sections at other depths in the sample. In embodiments, the imaging is accomplished by multi-photon microscopy (e.g., two-photon excited fluorescence or two-photon-pumped microscopy). Unlike conventional single-photon emission, multi-photon microscopy can utilize much longer excitation wavelength up to the red or near-infrared spectral region. This lower energy excitation requirement enables the implementation of semiconductor diode lasers as pump sources to significantly enhance the photostability of materials. Scanning a single focal point across the field of view is likely to be too slow for many sequencing applications. To speed up the image acquisition, an array of multiple focal points can be used. The emission from each of these focal points can be imaged onto a detector, and the time information from the scanning mirrors can be translated into image coordinates. Alternatively, the multiple focal points can be used just for the purpose of confining the fluorescence to a narrow axial section, and the emission can be imaged onto an imaging detector, such as a CCD, EMCCD, or s-CMOS detector. A scientific grade CMOS detector offers an optimal combination of sensitivity, readout speed, and low cost. One configuration used for confocal microscopy is spinning disk confocal microscopy. In 2-photon microscopy, the technique of using multiple focal points simultaneously to parallelize the readout has been called Multifocal Two-Photon Microscopy (MTPM). Several techniques for MTPM are available, with applications typically involving imaging in biological tissue. In embodiments of the methods provided herein, the imaging is accomplished by light sheet fluorescence microscopy (LSFM). In embodiments, detecting includes 3D structured illumination (3DSIM). In 3DSIM, patterned light is used for excitation, and fringes in the Moiré pattern generated by interference of the illumination pattern and the sample, are used to reconstruct the source of light in three dimensions. In order to illuminate the entire field, multiple spatial patterns are used to excite the same physical area, which are then digitally processed to reconstruct the final image. See York, Andrew G., et al. “Instant super-resolution imaging in live cells and embryos via analog image processing.” Nature methods 10.11 (2013): 1122-1126 which is incorporated herein by reference. In embodiments, detecting includes selective planar illumination microscopy, light sheet microscopy, emission manipulation, pinhole confocal microscopy, aperture correlation confocal microscopy, volumetric reconstruction from slices, deconvolution microscopy, or aberration-corrected multifocus microscopy. In embodiments, detecting includes digital holographic microscopy (see for example Manoharan, V. N. Frontiers of Engineering: Reports on Leading-edge Engineering from the 2009 Symposium, 2010, 5-12, which is incorporated herein by reference). In embodiments, detecting includes confocal microscopy, light sheet microscopy, or multi-photon microscopy.

In embodiments, the method further includes sequencing the amplification product (e.g., sequencing the amplification product of step (f)). In embodiments, sequencing includes sequencing by synthesis, sequencing by binding, sequencing by ligation, or pyrosequencing. In embodiments, sequencing includes sequencing by synthesis, sequencing by ligation, or pyrosequencing. In embodiments, sequencing includes extending a sequencing primer by incorporating a labeled nucleotide, or labeled nucleotide analogue, and detecting the label to generate a signal for each incorporated nucleotide or nucleotide analogue, wherein the sequencing primer is hybridized to the extension product.

In embodiments, the method includes sequencing the amplification products, which includes the barcode sequence. A variety of sequencing methodologies can be used such as sequencing-by synthesis (SBS), pyrosequencing, sequencing by ligation (SBL), or sequencing by hybridization (SBH). Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11(1), 3-11 (2001); Ronaghi et al. Science 281(5375), 363 (1998); U.S. Pat. Nos. 6,210,891; 6,258,568; and 6,274,320, each of which is incorporated herein by reference in its entirety). In pyrosequencing, released PPi can be detected by being converted to adenosine triphosphate (ATP) by ATP sulfurylase, and the level of ATP generated can be detected via light produced by luciferase. In this manner, the sequencing reaction can be monitored via a luminescence detection system. In both SBL and SBH methods, target nucleic acids, and amplicons thereof, are subjected to repeated cycles of oligonucleotide delivery and detection. SBL methods, include those described in Shendure et al. Science 309:1728-1732 (2005); U.S. Pat. Nos. 5,599,675; and 5,750,341, each of which is incorporated herein by reference in its entirety; and the SBH methodologies are as described in Bains et al., Journal of Theoretical Biology 135(3), 303-7 (1988); Drmanac et al., Nature Biotechnology 16, 54-58 (1998); Fodor et al., Science 251(4995), 767-773 (1995); and WO 1989/10977, each of which is incorporated herein by reference in its entirety.

In SBS, extension of a nucleic acid primer along a nucleic acid template is monitored to determine the sequence of nucleotides in the template. The underlying chemical process can be catalyzed by a polymerase, wherein fluorescently labeled nucleotides are added to a primer (thereby extending the primer) in a template dependent fashion such that detection of the order and type of nucleotides added to the primer can be used to determine the sequence of the template. In embodiments, sequencing includes annealing and extending a sequencing primer to incorporate a detectable label that indicates the identity of a nucleotide in the target polynucleotide, detecting the detectable label, and repeating the extending and detecting of steps. In embodiments, the methods include sequencing one or more bases of a target nucleic acid by extending a sequencing primer hybridized to a target nucleic acid (e.g., an amplification product produced by the amplification methods described herein). In embodiments, sequencing may be accomplished by a sequencing-by-synthesis (SBS) process. In embodiments, sequencing includes a sequencing by synthesis process, where individual nucleotides are identified iteratively, as they are polymerized to form a growing complementary strand. In embodiments, nucleotides added to a growing complementary strand include both a label and a reversible chain terminator that prevents further extension, such that the nucleotide may be identified by the label before removing the terminator to add and identify a further nucleotide. Such reversible chain terminators include removable 3′ blocking groups, for example as described in U.S. Pat. Nos. 7,541,444 and 7,057,026. Once such a modified nucleotide has been incorporated into the growing polynucleotide chain complementary to the region of the template being sequenced, there is no free 3′-OH group available to direct further sequence extension and therefore the polymerase cannot add further nucleotides. Once the identity of the base incorporated into the growing chain has been determined, the 3′ reversible terminator may be removed to allow addition of the next successive nucleotide. By ordering the products derived using these modified nucleotides it is possible to deduce the DNA sequence of the oligonucleotide target nucleic acid sequence.

In embodiments, the methods of sequencing a nucleic acid include a extending a polynucleotide by using a polymerase. In embodiments, the polymerase is a DNA polymerase. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol μ DNA polymerase, Pol λ DNA polymerase, Pol σ DNA polymerase, Pol α DNA polymerase, Pol δ DNA polymerase, Pol ε DNA polymerase, Pol η DNA polymerase, Pol ι DNA polymerase, Pol κ DNA polymerase, Pol ζ DNA polymerase, Pol γ DNA polymerase, Pol θ DNA polymerase, Pol ν DNA polymerase, or a thermophilic nucleic acid polymerase (e.g., Therminator γ, 9° N polymerase (exo-), Therminator II, Therminator III, or Therminator IX). In embodiments, the DNA polymerase is a thermophilic nucleic acid polymerase. In embodiments, the DNA polymerase is a modified archacal DNA polymerase. In embodiments, the polymerase is a mutant P. abyssi polymerase (e.g., such as a mutant P. abyssi polymerase described in WO 2018/148723 or WO 2020/056044, each of which are incorporated herein by reference for all purposes). In embodiments, the polymerase is a bacterial DNA polymerase, eukaryotic DNA polymerase, archacal DNA polymerase, viral DNA polymerase, or phage DNA polymerases. Bacterial DNA polymerases include E. coli DNA polymerases I, II and III, IV and V, the Klenow fragment of E. coli DNA polymerase, Clostridium stercorarium (Cst) DNA polymerase, Clostridium thermocellum (Cth) DNA polymerase and Sulfolobus solfataricus (Sso) DNA polymerase. Eukaryotic DNA polymerases include DNA polymerases α, β, γ, δ, Ε, η, ζ, λ, σ, u, and k, as well as the Revl polymerase (terminal deoxycytidyl transferase) and terminal deoxynucleotidyl transferase (TdT). Viral DNA polymerases include T4 DNA polymerase, phi-29 DNA polymerase, GA-1, phi-29-like DNA polymerases, PZA DNA polymerase, phi-15 DNA polymerase, Cpl DNA polymerase, Cpl DNA polymerase, T7 DNA polymerase, and T4 polymerase. Other useful DNA polymerases include thermostable and/or thermophilic DNA polymerases such as Thermus aquaticus (Taq) DNA polymerase, Thermus filiformis (Tfi) DNA polymerase, Thermococcus zilligi (Tzi) DNA polymerase, Thermus thermophilus (Tth) DNA polymerase, Thermus flavusu (Tfl) DNA polymerase, Pyrococcus wocsci (Pwo) DNA polymerase, Pyrococcus furiosus (Pfu) DNA polymerase and Turbo Pfu DNA polymerase, Thermococcus litoralis (Tli) DNA polymerase, Pyrococcus sp. GB-D polymerase, Thermotoga maritima (Tma) DNA polymerase, Bacillus stearothermophilus (Bst) DNA polymerase, Pyrococcus Kodakaraensis (KOD) DNA polymerase, Pfx DNA polymerase, Thermococcus sp. JDF-3 (JDF-3) DNA polymerase, Thermococcus gorgonarius (Tgo) DNA polymerase, Thermococcus acidophilium DNA polymerase; Sulfolobus acidocaldarius DNA polymerase; Thermococcus sp. go N-7 DNA polymerase; Pyrodictium occultum DNA polymerase; Methanococcus voltae DNA polymerase; Methanococcus thermoautotrophicum DNA polymerase; Methanococcus jannaschii DNA polymerase; Desulfurococcus strain TOK DNA polymerase (D. Tok Pol); Pyrococcus abyssi DNA polymerase; Pyrococcus horikoshii DNA polymerase; Pyrococcus islandicum DNA polymerase; Thermococcus fumicolans DNA polymerase; Acropyrum pernix DNA polymerase; and the heterodimeric DNA polymerase DP1/DP2. In embodiments, the polymerase is 3PDX polymerase as disclosed in U.S. Pat. No. 8,703,461, the disclosure of which is incorporated herein by reference. In embodiments, the polymerase is a reverse transcriptase. Exemplary reverse transcriptases include, but are not limited to, HIV-1 reverse transcriptase from human immunodeficiency virus type 1 (PDB 1HMV), HIV-2 reverse transcriptase from human immunodeficiency virus type 2, M-MLV reverse transcriptase from the Moloney murine leukemia virus, AMV reverse transcriptase from the avian myeloblastosis virus, and Telomerase reverse transcriptase.

In embodiments, sequencing includes a plurality of sequencing cycles. In embodiments, sequencing includes 20 to 100 sequencing cycles. In embodiments, sequencing includes 50 to 100 sequencing cycles. In embodiments, sequencing includes 50 to 300 sequencing cycles. In embodiments, sequencing includes 50 to 150 sequencing cycles. In embodiments, sequencing includes at least 10, 20, 30 40, or 50 sequencing cycles. In embodiments, sequencing includes at least 10 sequencing cycles. In embodiments, sequencing includes 10 to 20 sequencing cycles. In embodiments, sequencing includes 10, 11, 12, 13, 14, or 15 sequencing cycles. In embodiments, sequencing includes (a) extending a sequencing primer by incorporating a labeled nucleotide, or labeled nucleotide analogue and (b) detecting the label to generate a signal for each incorporated nucleotide or nucleotide analogue.

In embodiments, sequencing includes sequentially extending a plurality of sequencing primers (e.g., sequencing a first region of a target nucleic acid followed by sequencing a second region of a target nucleic acid, followed by sequencing N regions, where N is the number of sequencing primers in the known sequencing primer set). In embodiments, sequencing includes generating a plurality of sequencing reads.

In embodiments, sequencing includes extending a sequencing primer to generate a sequencing read. In embodiments, sequencing includes extending a sequencing primer by incorporating a labeled nucleotide, or labeled nucleotide analogue and detecting the label to generate a signal for each incorporated nucleotide or nucleotide analogue. In embodiments, the labeled nucleotide or labeled nucleotide analogue further includes a reversible terminator moiety. In embodiments, the labeled nucleotide or labeled nucleotide analogue further includes a reversible terminator moiety. In embodiments, the reversible terminator moiety is attached to the 3′ oxygen of the nucleotide and is independently

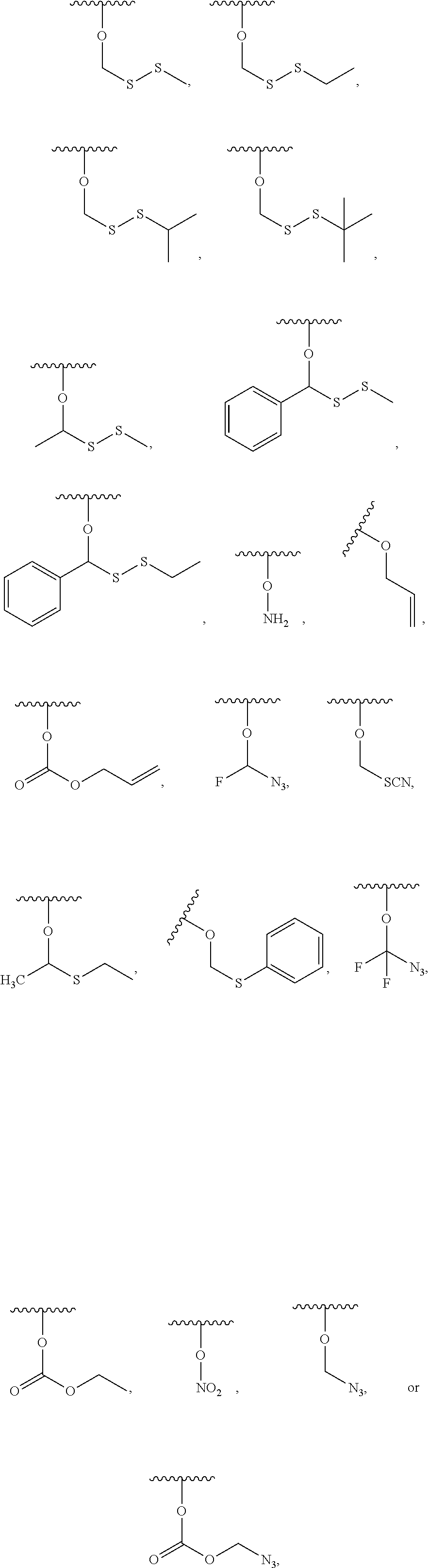

wherein the 3′ oxygen is explicitly depicted in the above formulae. Additional examples of reversible terminators may be found in U.S. Pat. No. 6,664,079, Ju J. et al. (2006) Proc Natl Acad Sci USA 103(52): 19635-19640; Ruparel H. et al. (2005) Proc Natl Acad Sci USA 102(17):5932-5937; Wu J. et al. (2007) Proc Natl Acad Sci USA 104(104):16462-16467; Guo J. et al. (2008) Proc Natl Acad Sci USA 105(27): 9145-9150 Bentley D. R. et al. (2008) Nature 456(7218):53-59; or Hutter D. et al. (2010) Nucleosides Nucleotides & Nucleic Acids 29:879-895, which are incorporated herein by reference in their entirety for all purposes. In embodiments, a polymerase-compatible cleavable moiety includes an azido moiety or a dithiol moiety.

In embodiments, the method includes sequencing a plurality of target polynucleotides of a cell in situ within an optically resolved volume. In embodiments, the number of unique targets detected within an optically resolved volume of a sample is about 3, 10, 30, 50, or 100. In embodiments, the number of unique targets detected within an optically resolved volume of a sample is about 1 to 10. In embodiments, the number of unique targets detected within an optically resolved volume of a sample is about 5 to 10. In embodiments, the number of unique targets detected within an optically resolved volume of a sample is about 1 to 5. In embodiments, the number of unique targets detected within an optically resolved volume of a sample is at least 3, 10, 30, 50, or 100. In embodiments, the number of unique targets detected within an optically resolved volume of a sample is less than 3, 10, 30, 50, or 100. In embodiments, the number of unique targets detected within an optically resolved volume of a sample is about 1, 2, 3, 4, 5, 6, 7, 8, 9, 10, 20, 50, 100, 500, 1,000, 5,000, 10,000, or 200,000. In embodiments, the methods allow for detection of a single target of interest. In embodiments, the methods allow for multiplex detection of a plurality of targets of interest.

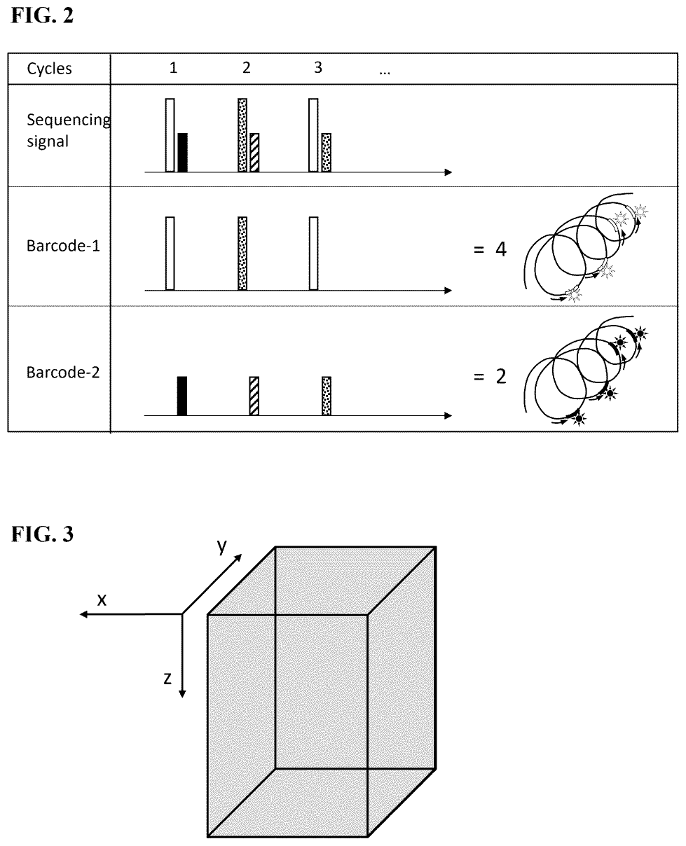

In embodiments, the optically resolved volume has an axial resolution (i.e., depth, or z) that is greater than the lateral resolution (i.e., xy plane). In embodiments, the optically resolved volume has an axial resolution that is greater than twice the lateral resolution. In embodiments, the dimensions (i.e., the x, y, and z dimensions) of the optically resolved volume are about 0.5 μm×0.5 μm×0.5 μm; 1 μm×1 μm×1 μm; 2 μm×2 μm×2 μm; 0.5 μm×0.5 μm×1 μm; 0.5 μm×0.5 μm×2 μm; 2 μm×2 μm×1 μm; or 1 μm×1 μm×2 μm. In embodiments, the dimensions (i.e., the x, y, and z dimensions) of the optically resolved volume are about 1 μm×1 μm×2 μm; 1 μm×1 μm×3 μm; 1 μm×1 μm×4 μm; or about 1 μm×1 μm×5 μm. See FIG. 2, for example. In embodiments, the dimensions (i.e., the x, y, and z dimensions) of the optically resolved volume are about 1 μm×1 μm×5 μm. In embodiments, the dimensions (i.e., the x, y, and z dimensions) of the optically resolved volume are about 1 μm×1 μm×6 μm. In embodiments, the dimensions (i.e., the x, y, and z dimensions) of the optically resolved volume are about 1 μm×1 μm×7 μm. In embodiments, the optically resolved volume is a cubic micron. In embodiments, the optically resolved volume has a lateral resolution from about 100 to 200 nanometers, from 200 to 300 nanometers, from 300 to 400 nanometers, from 400 to 500 nanometers, from 500 to 600 nanometers, or from 600 to 1000 nanometers. In embodiments, the optically resolved volume has an axial resolution from about 100 to 200 nanometers, from 200 to 300 nanometers, from 300 to 400 nanometers, from 400 to 500 nanometers, from 500 to 600 nanometers, or from 600 to 1000 nanometers. In embodiments, the optically resolved volume has an axial resolution from about 1 to 2 μm, from 2 to 3 μm, from 3 to 4 μm, from 4 to 5 μm, from 5 to 6 μm, or from 6 to 10 μm.

In embodiments, the method further includes an additional imaging modality, immunofluorescence (IF), or immunohistochemistry modality (e.g., immunostaining). In embodiments, the method includes ER staining (e.g., contacting the cell with a cell-permeable dye which localizes to the endoplasmic reticula), Golgi staining (e.g., contacting the cell with a cell-permeable dye which localizes to the Golgi), F-actin staining (e.g., contacting the cell with a phalloidin-conjugated dye that binds to actin filaments), lysosomal staining (e.g., contacting the cell with a cell-permeable dye that accumulates in the lysosome via the lysosome pH gradient), mitochondrial staining (e.g., contacting the cell with a cell-permeable dye which localizes to the mitochondria), nucleolar staining, or plasma membrane staining. For example, the method includes live cell imaging (e.g., obtaining images of the cell) prior to or during fixing, immobilizing, and permeabilizing the cell. Immunohistochemistry (IHC) is a powerful technique that exploits the specific binding between an antibody and antigen to detect and localize specific antigens in cells and tissue, commonly detected and examined with the light microscope. Known IHC modalities may be used, such as the protocols described in Magaki, S., Hojat, S. A., Wei, B., So, A., & Yong, W. H. (2019). Methods in molecular biology (Clifton, N.J.), 1897, 289-298, which is incorporated herein by reference. In embodiments, the additional imaging modality includes bright field microscopy, phase contrast microscopy, Nomarski differential-interference-contrast microscopy, or dark field microscopy. In embodiments, the method further includes determining the cell morphology (e.g., the cell boundary or cell shape) using known methods in the art. For example, to determining the cell boundary includes comparing the pixel values of an image to a single intensity threshold, which may be determined quickly using histogram-based approaches as described in Carpenter, A. et al Genome Biology 7, R100 (2006) and Arce, S., Sci Rep 3, 2266 (2013)).

In embodiments, the barcodes in the known set of barcodes have a specified Hamming distance. In embodiments, the Hamming distance is 4 to 15. In embodiments, the Hamming distance is 8 to 12. In embodiments, the Hamming distance is 10. In embodiments, the Hamming distance is 0 to 100. In embodiments, the Hamming distance is 0 to 15. In embodiments, the Hamming distance is 0 to 10. In embodiments, the Hamming distance is 1 to 10. In embodiments, the Hamming distance is 5 to 10. In embodiments, the Hamming distance is 1 to 100. In embodiments, the Hamming distance between any two barcode sequences of the set is at least 2, 3, 4, or 5. In embodiments, the Hamming distance between any two barcode sequences of the set is at least 3. In embodiments, the Hamming distance between any two barcode sequences of the set is at least 4.

In embodiments, demultiplexing the multiplexed signal includes a linear decomposition of the multiplexed signal. Any of a variety of techniques may be employed for decomposition of the multiplexed signal. Examples include, but are not limited to, Zimmerman et al. Chapter 5: Clearing Up the Signal: Spectral Imaging and Linear Unmixing in Fluorescence Microscopy; Confocal Microscopy: Methods and Protocols, Methods in Molecular Biology, vol. 1075 (2014); Shirawaka H. et al.; Biophysical Journal Volume 86, Issue 3, March 2004, Pages 1739-1752; and S. Schlachter, et al, Opt. Express 17, 22747-22760 (2009); the content of each of which is incorporated herein by reference in its entirety. In embodiments, multiplexed signal includes overlap of a first signal and a second signal and is computationally resolved, for example, by imaging software.

In embodiments, the method further includes measuring an amount of one or more of the targets by counting the one or more associated barcodes. In embodiments, the method further includes counting the one or more associated barcodes in an optically resolved volume.

In embodiments, the number of unique targets detected within an optically resolved volume of a sample is about 3, 10, 30, 50, or 100. In embodiments, the number of unique targets detected within an optically resolved volume of a sample is about 1 to 10. In embodiments, the number of unique targets detected within an optically resolved volume of a sample is about 5 to 10. In embodiments, the number of unique targets detected within an optically resolved volume of a sample is about 1 to 5. In embodiments, the number of unique targets detected within an optically resolved volume of a sample is at least 3, 10, 30, 50, or 100. In embodiments, the number of unique targets detected within an optically resolved volume of a sample is less than 3, 10, 30, 50, or 100. In embodiments, the number of unique targets detected within an optically resolved volume of a sample is about 1, 2, 3, 4, 5, 6, 7, 8, 9, 10, 20, 50, 100, 500, 1,000, 5,000, 10,000, or 200,000. In embodiments, the methods allow for detection of a single target of interest. In embodiments, the methods allow for multiplex detection of a plurality of targets of interest. The use of oligonucleotide barcodes with unique identifier sequences as described herein allows for simultaneous detection of 2, 3, 4, 5, 6, 7, 8, 9, 10, 20, 30, 40, 50, 60, 70, 80, 90, 100, 200, 300, 400, 500, 600, 700, 800, 900, 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, 10,000 or more than 10,000 unique targets within a single cell. In contrast to existing in situ detection methods, the methods presented herein have the advantage of virtually limitless numbers of individually detected molecules in parallel and in situ.

In embodiments, the total volume of the cell is about 1 to 25 μm³. In embodiments, the volume of the cell is about 5 to 10 μm³. In embodiments, the volume of the cell is about 3 to 7 μm³.

In aspects and embodiments described herein, the methods are useful in the field of predictive medicine in which diagnostic assays, prognostic assays, pharmacogenomics, and monitoring clinical trials are used for prognostic (i.e., predictive) purposes to thereby treat an individual prophylactically. Accordingly, in embodiments the methods of diagnosing and/or prognosing one or more diseases and/or disorders using one or more of expression profiling methods described herein are provided.

In an aspect is provided a method of detecting a disorder (e.g., cancer) or a disease-causing mutation or allele in a cell. In embodiments, the cell includes an oncogene (e.g., HER2, BRAF, EGFR, KRAS) and utilizing the methods described herein the oncogene is identified, thereby detecting a disorder when the presence of the oncogene is identified. In embodiments, the sample includes a nucleic acid molecule which includes a disease-causing mutation or allele. In embodiments, the method includes hybridizing an oligonucleotide primer which is correlated with the disease-causing mutation or allele. In embodiments, the method includes ligating a mutation-specific oligonucleotide probe only when the disease-causing mutation or allele is present in the nucleic acid target. In embodiments, the disease-causing mutation or allele is a base substitution, an insertion mutation, a deletion mutation, a gene amplification, a gene deletion, a gene fusion event, or a gene inversion event.

In embodiments, the mutation or allele is associated with an increased predisposition for one or more diseases, disorders, or other phenotypes. In embodiments, the mutation or allele is associated with a decreased predisposition for one or more diseases, disorders, or other phenotypes. For example, some mutations or alleles are associated with a cancer phenotype, such as decreased growth inhibition, evasion of immune detection, or dedifferentiation. Mutations that can be detected using the method provided herein include for example, mutations to BRAF, EGFR, Her2/ERBB2, and other somatic mutations as exemplified by Greenman et al., Nature (2007) 446:153-158, hereby incorporated by reference in its entirety.

### EXAMPLES

### Example 1. In Situ Transcriptomics

A wealth of information is reflected in the temporal and spatial variation of gene and protein expression among cells. Cellular macromolecules such as nucleic acids and proteins, occupy precise positions in cells and tissues, and a great deal of information is lost when these molecules are extracted. The methods available today for RNA sequence analysis (RNA-Seq) have the capacity to quantify the abundance of RNA molecules in a population of cells with great sensitivity. Current methods for single-cell RNA and protein analysis typically involve some method for “barcoding” the content of individual cells, followed by pooling the content and sequencing on a commercial DNA sequencing device (e.g., an Illumina or Singular Genomics G4™ sequencing platform). The barcoding can be done in individual wells on a microplate (e.g., a microplate with 96, 384, or 1536 wells), and more recently droplet-based methods are emerging as an essential tool for single-cell genomics research (see for example, Klein A. and Macosko E. Lab Chip. 2017; 17(15):2540-2541; and Zheng, G. X., et al. Nature communications, 2017; 8, 14049). Briefly, droplet-based methods begin with isolating a cell from a sample (e.g., a tissue) and encapsulating the cell in a droplet where unique identifying oligonucleotides (i.e., barcodes) are incorporated into the genomic sequence, often while converting RNAs to cDNAs during reverse transcription. These barcodes uniquely label the cDNA and identify the cellular origin. The cDNAs are then extracted and undergo standard library preparation for sequencing before being sequenced on a commercial sequencer. mRNA expression is then quantified by counting the number of barcodes that mapped to each cell.

These methods have found wide application dissecting transcriptomic heterogencity, and can handle upwards of 10,000 cells in an automated format, however they have several limitations and drawbacks. For example, if the cells of interest originate from a tissue sample, all information about the spatial distribution of the cells within the tissue is lost in the process of dissociating and isolating the cells prior to barcoding them. Often information about the intracellular distribution of analytes within the cellular microenvironment is also lost. This information can be vital to designing therapeutic approaches to cancers, for example, where the tumor microenvironment often creates spatial gradients of nutrients and metabolic byproducts. Droplet-based techniques are capable of barcoding and sequencing tens of thousands of cells (e.g., 10-50 thousand cells) in a single experiment but current approaches require generation of custom microfluidic devices, reagents, and sample preparation techniques (e.g., as found in the disclosures U.S. Pat. No. RE41,780 and US 2015/0225778). Additionally, due to the digital “counting” nature of the sequencing readout, hundreds of sequencing reads/cell are required to get information about the expression of less abundant genes. For example, if a particular abundant gene is transcribed into 500 copies of RNA, the abundant gene will dominate the sequencing run resulting in relatively inefficient use of sequencing capacity. However, cells can associate with multiple barcodes which significantly impacts single-cell analyses and rare cell events (Lareau, C. A., et al. (2020) Nature communications, 11(1), 866).

A different barcoding approach has been applied to spatial profiling of RNA & proteins in tissue. An example of this is the method developed by Spatial Transcriptomics, a Stockholm-based company purchased by 10X Genomics in 2018 and recently commercialized as “Visium Spatial” platform. This approach involves attaching a section of a frozen tissue of interest to patterned microarrays carrying spatially barcoded oligo-dT primers that capture the entire polyadenylated transcriptome contained in the tissue section. Each spot on the microarray contains a capture probe with a spatial barcode unique to that spot allowing the individual sequencing reads to be mapped to the originating spot. After cDNA synthesis on the surface via reverse transcription, the tissue is removed and the mRNA-cDNA hybrids are released from the array to be prepared for sequencing; see Vickovic, S., et al. Nat. Methods 16, 987-990 (2019) for greater detail on the approach. The current implementation of this technology includes a microarray with 100 μm spots spaced equidistant from each other, approximately 200 μm apart. The spatial resolution of this method is approximately 100 μm, which is sufficient for a coarse mapping of a pathology sample, but is insufficient to resolve individual cells, which are approximately 10-20 μm, or subcellular features (i.e., features less than 10 μm, such as the mitochondria). Wide adoption of this approach has been limited by the lack of scalability and accessible ways to automate and/or parallelize sequencing library preparation.

A number of new techniques have been described for reading out RNA transcription levels in tissue sections directly (i.e., in-situ), without requiring spatial barcoding, based on single molecule fluorescence in situ hybridization. These include MERFISH (Multiplexed Error-Robust Fluorescence In Situ Hybridization), STARmap (Spatially-resolved Transcript Amplicon Readout mapping), DART-FISH, seq-FISH (Sequential Fluorescence In Situ Hybridization) and others (see for example Chen, K. H., et al. (2015). Science, 348(6233), aaa6090; Wang, G., Moffitt, J. R. & Zhuang, X. Sci Rep. 2018; 8, 4847; Wang X. et al; Science, 2018; 27, Vol 361, Issue 6400, caat5691; Cai, M. Dissertation, (2019) UC San Diego. ProQuest ID: Cai_ucsd_0033D_18822; and Sansone, A. Nat Methods 16, 458; 2019). In all of these techniques, individual RNA transcripts are individually resolved, typically with pre-amplification or requiring multiple instances of labeled probes. Some of these techniques have been combined with super-resolution microscopy, expansion microscopy, or both, to increase the resolution and allow more transcripts to be resolved and thus counted. This increases the complexity and costs of detection, and can require laborious sample preparation and time consuming wash protocols.

Gap fill padlock probes (PLPs) are extremely useful when there is an unknown region on a target RNA or DNA molecule flanked by conserved and known sequences (e.g., a left flanking region and a right flanking region). Typically, gap fill PLPs include a first hybridization pad (i.e., a first hybridization hybridization complementary to a first target sequence) and a second hybridization pad (i.e., a second hybridization hybridization complementary to a second target sequence) which can each be directly targeted, for example, to the conserved and known sequences surrounding an unknown sequence. By polymerizing (e.g., with a DNA polymerase) the gap between the two hybridization pads, the reverse complement of the unknown sequence is incorporated into the PLP, which, upon ligation, becomes a circular template for rolling circle amplification (RCA).

Described herein are methods for addressing these and other problems in the art. An aspect of the invention is to allow the readout of multiple RNA transcripts using a set of oligonucleotide probes (e.g., up to 48 or more per gene), wherein each probe contains a unique barcode that specific for the particular sequence being targeted. A pair of third and second oligonucleotide probes are then hybridized to the first and second oligonucleotides (e.g., the RNA targeting probes), wherein the same third and second oligonucleotide probes are used for each pair of RNA targeting probes (e.g., universal third and second oligonucleotide probes). The method includes targeting specific RNA sequences, and “translating” them to a DNA barcode, with a means for local amplification. The method includes selecting barcodes that are widely spaced in the combinatorial space of possible barcodes (large Hamming distance). The method includes sequencing the barcodes. The methods described herein (for example within the aspects and embodiments) reveal the distribution of specific RNA molecules in cells and tissues. In this way, patterns of differential gene expression may be observed which aids in the understanding of a particular gene's function, and ultimately the phenotype of the cell.

### Example 2. Nucleic Acid Detection with Multi-Part Oligonucleotide Probes

Single-molecule RNA FISH (smFISH) is a versatile assay that allows detecting single RNA molecules with high specificity. In smFISH, a set of typically 30-50 oligonucleotides of 20 nucleotides (nt), each conjugated to a single fluorophore, are hybridized to a complementary RNA target. Individual transcripts are then visualized as diffraction-limited spots using wide-field epifluorescence microscopy, and quantified. A major limitation of this approach, however, is that smFISH signals are dim, while background fluorescence is high in FFPE samples (with large variability depending on the tissue type, sample age, and fixation conditions). Hence, imaging at high magnification (60-100×) is required. As a result, only a very small area of the sample is usually imaged (typically, 40-50 fields of view), making it difficult to assess intratumor heterogeneity.

In contrast to smFISH, other methods such as RNAscope, and single-molecule hybridization chain reaction (smHCR) involve one or more steps of signal amplification, which results in brighter fluorescence signals and higher signal-to-noise ratio. However, these methods have a number of drawbacks that limit their utility for the purpose of quantifying the intratumor heterogeneity of clinically relevant biomarkers. The sensitivity of RNAscope is lower compared to smFISH. Fluorescent in situ RNA Sequencing (FISSEQ) is another approach in which RCA products (RCPs) are generated from self-circularized cDNA in a non-targeted fashion, and then sequenced in situ by sequencing-by-ligation (SBL). First, cDNA synthesis is performed using a mix of regular and modified amine-bases, together with tagged random-hexamer RT primers. Via the amine bases, cDNA is cross-linked to its cellular environment and thereafter circularized by ligation. Subsequent RCA creates single stranded DNA nanoballs, whose positions are maintained via cross-linking to the cellular protein matrix. Sequencing is then performed using SBL with a read-length of 30 bases. However, to discriminate many different targets, a partition sequencing strategy is applied by using extended sequencing primers, where nanoballs are randomly sampled so that only a subset is sequenced (see, Asp M et al. Bioessays. 2020; 42(10):e1900221, which is incorporated herein by reference in its entirety).

Recently, a new assay termed RollFISH was developed that integrates the specificity of smFISH with rolling circle amplification (see, e.g., Wu C et al. Commun. Biol. 2018; 1:209, which is incorporated herein by reference in its entirety). In RollFISH, the design of smFISH probes is modified so that padlock probes can be docked to them, thus enabling signal amplification. Briefly, each RollFISH probe consists of a set of oligonucleotide probes, each containing a 30 nt sequence complementary to the RNA target, followed by a 46 nt docking sequence orthogonal to the human transcriptome on the 3′ end. The oligonucleotide probes are hybridized in situ to their complementary target, followed by removal of unspecifically bound ODNs. Next, a padlock probe containing a transcript-specific barcode sequence is docked to each oligonucleotide probe and circularized in situ to form a single-stranded DNA circle. As in standard RCA, rolling circle amplification is then carried out using the Phi29 polymerase primed by the 3′ end of the oligonucleotide probe. The resulting RCA product (RCP) contains hundreds to thousands of copies of the reverse sequence of the transcript-specific barcode sequence, which is detected using a fluorescently labeled secondary probe. Because a RollFISH probe consists of many oligonucleotide probes, multiple RCPs can form simultaneously on the same transcript molecule, resulting in a bright fluorescent spot that can be visualized at low magnification (20×) using widefield epifluorescence microscopy.

A drawback of the RollFISH design is that the individual padlock probes include the barcode sequence, and thus a unique padlock probe must be designed for each region of nucleic acid of interest being targeted by the oligonucleotide probes. Padlock probes are typically longer oligonucleotides, and thus generating a panel of unique probe for transcriptome detection is an expensive process. An alternate method for detection of target RNA in a sample was recently described that uses a hairpin probe which unfolds upon binding to its target RNA sequence, revealing a 3′ end region which is capable of binding to a circularizable probe (sec, U.S. Pat. Pub. US 2022/0010348, which is incorporated herein by reference in its entirety). The 3′ end region (i.e., the region that binds to a circularizable probe) may include a gap fill sequence for incorporation into the circularizable probe. The hairpin probe is also described as including a reporter domain used to distinguish between different target sequences in a target RNA, wherein the reporter domain is in the region of complementarity between the hairpin probe and the circularizable probe, such that a different circularizable probe is required to detect each reporter domain in the hairpin probe

When targeting known regions on a target RNA or DNA molecule, it is possible to design targeted oligonucleotide probes that include a single hybridization sequence complementary to the known nucleic acid sequence, resulting in a shorter oligonucleotide, in comparison with a gap fill PLP that includes two hybridization sequences. Utilizing the methods described herein, efficient and comprehensive in situ sequencing of diverse target populations using a single gap fill PLP may be realized. An alternative approach is presented herein wherein a pair (i.e., two) of probes are used to target a nucleic acid sequence of interest. Using a pair of probes for the initial target nucleic acid binding step may confer additional specificity over the use of a single target oligonucleotide probe, for example, under higher stringency hybridization conditions. The use of a first and second oligonucleotide each with regions of complementarity to a third and second oligonucleotide probe (i.e., the two circularization probes) also increases the specificity of the hybridization, as each of the third and second oligonucleotide probes must hybridize to both the first and second oligonucleotides in order to generate a circular oligonucleotide including a barcode sequence corresponding to the target nucleic acid of interest. By using targeted oligonucleotide probes wherein the first and second probe hybridization sequences and barcode sequence consist primarily of DNA, high efficiency ligation and circularization is achieved, leading to increased amplification product formation. The present methods include the advantage of not having to perform reverse transcription of a target RNA molecule to generate cDNA, for example, as the polynucleotide probe can directly hybridize to RNA.

In situ sequencing involves tissue and/or cellular extraction, combined with the fixation and permeabilization of cells, followed by amplification of the target nucleic acid fragments for sequencing. Briefly, cells and their surrounding milieu are attached to a substrate surface, fixed, and permeabilized. Targeted oligonucleotide probes designed for RNA detection are then annealed to nucleic acid regions of interest. For example, mRNA is targeting with a set of oligonucleotide probe targeting one or more regions of interest (e.g., up to 24, or up to 48 regions per gene). FIG. 4A illustrates an embodiment for detecting a target polynucleotide including a first oligonucleotide (11) and a second oligonucleotide (12), wherein the first oligonucleotide includes a first target hybridization sequence at a 3′ end (13), a first hybridization sequence (14; e.g., a sequence complementary to a second oligonucleotide probe as described in FIG. 4B), a barcode sequence (15), and a second hybridization sequence (16; e.g., a sequence complementary to a first oligonucleotide probe as described in FIG. 4B), and wherein the second oligonucleotide (12) includes a second target hybridization sequence at a 5′ end (17), a third hybridization sequence (18; e.g., a sequence complementary to the second oligonucleotide probe as described in FIG. 4B), and a fourth hybridization sequence (19; e.g., a sequence complementary to the first oligonucleotide probe as described in FIG. 4B). In some embodiment, the 3′ ends of the first and second oligonucleotides are blocked (e.g., reversibly blocked, for example with a ddNTP, to prevent nucleotide incorporation by a polymerase).

FIG. 4B illustrates a first oligonucleotide probe (20) and a second oligonucleotide probe (21), wherein the first oligonucleotide probe includes a sequence at a 3′ end (22) complementary to the fourth hybridization sequence of the second oligonucleotide (19) and a sequence at a 5′ end (23) complementary to the second hybridization sequence of the first oligonucleotide (16) as in FIG. 4A, and wherein the second oligonucleotide probe includes a sequence at a 5′ end (24) complementary to the third hybridization sequence of the second oligonucleotide (18) and a sequence at a 3′ end (25) complementary to the first hybridization sequence of the first oligonucleotide (14) as in FIG. 4A. A linker sequence may be present, as illustrated in FIG. 4B, between the first hybridization sequence complement and the third hybridization sequence complement (26), and between the second hybridization sequence complement and the fourth hybridization sequence complement (27).

FIG. 5A illustrates a first set of three independently targeted pairs of oligonucleotides (e.g., first and second oligonucleotides as described in FIG. 4A) hybridized to a first mRNA (28) and a second set of three independently targeted pairs of oligonucleotides hybridized to a second mRNA (29), wherein for each set, the first oligonucleotide of each pair includes a unique barcode (e.g., 30 or 31). Once each pair of first and second oligonucleotides are hybridized to the target regions (e.g., via each adjacent Target Sequence), a first oligonucleotide probe and a second oligonucleotide probe (as described in FIG. 4B) is hybridized to each pair of first and second oligonucleotides, as shown in FIG. 5B, wherein the 5′ end of the hybridized first oligonucleotide probe (23) and the 3′ end of the hybridized second oligonucleotide probe (25) flanks the barcode sequence (30 or 31) of the first oligonucleotide. A gap (e.g., a nick) is also present between the 3′ end of the first oligonucleotide probe (22) and the 5′ end of the second oligonucleotide probe (24).

Gap-filling is then performed with a polymerase to incorporate a complementary barcode sequence (38 or 39) into the 3′ end of the second oligonucleotide probe, as shown in FIG. 5C, followed by ligation of the nick and the complementary barcode sequence to the first oligonucleotide probe to generate a circular oligonucleotide. Following gap filling and ligation, this circular oligonucleotide may be amplified, for example, by rolling circle amplification (RCA) using a strand-displacing polymerase (40) and the 3′ end of the second oligonucleotide as an amplification primer, as shown in FIG. 5D. In embodiments where the 3′ end of the second oligonucleotide includes a cleavable blocking moiety, the blocking moiety is removed prior to amplification (e.g., removed via cleavage with a cleaving agent).

The third and second oligonucleotide probes may also be provided hybridized to each pair of first and second oligonucleotides prior to contacting a sample. In this approach, following hybridization of the first and second oligonucleotides to the target nucleic acid sequence, gap-filling extension is performed without an intervening third and fourth oligonucleotide hybridization step. Alternatively, gap-filling extension and ligation to incorporate a complement of the barcode sequence into the second oligonucleotide probe may also be performed prior to contacting each pair of first and second oligonucleotides to a sample. In this embodiment, for example, each pair of first and second oligonucleotides would be hybridized to a pair of third and second oligonucleotide probes prior to hybridizing to a target nucleic acid in the sample.

Optionally, the RCA reaction can be done with modified nucleotides that contain chemical groups that serve as attachment points to the cell or the matrix in which the cell is embedded (e.g., a hydrogel). The attachment of the amplified product to the matrix can help confine & fix the amplicon to a small volume. In embodiments, amplification reactions include standard dNTPs and a modified nucleotide (e.g., amino-allyl dUTP, 5-TCO-PEG4-dUTP, C8-Alkyne-dUTP, 5-Azidomethyl-dUTP, 5-Vinyl-dUTP, or 5-Ethynyl dLTTP). For example, during amplification a mixture of standard dNTPs and aminoallyl deoxyuridine 5′-triphosphate (dUTP) nucleotides may be incorporated into the amplicon and subsequently cross-linked to the cell protein matrix by using a cross-linking reagent (e.g., an amine-reactive crosslinking agent with PEG spacers, such as (PEGylated bis(sulfosuccinimidyl)suberate) (BS(PEG)9)).

Optionally, one or more nucleotides within the amplification primer sequence, the sequencing primer sequence, and/or the immobilized oligonucleotide primer contains one or more functional moieties (e.g., bioconjugate reactive groups) that serve as attachment points to the cell or the matrix in which the cell is embedded (e.g., a hydrogel). In embodiments, one or more nucleotides within the amplification primer sequence, the sequencing primer sequence, and/or the immobilized oligonucleotide primer contains one or more functional moieties (e.g., bioconjugate reactive groups) that serve as attachment points to complementary bioconjugate reactive groups within the cell (e.g., a protein). In embodiments, a plurality of oligonucleotide primers are provided to the matrix in which the cell is embedded prior to amplification. In embodiments, a plurality of oligonucleotide primers are provided to the cell or the matrix in which the cell is embedded concurrently with amplification. In embodiments, the bioconjugate reactive group is located at the 5′ or 3′ end of the primer. In embodiments, the bioconjugate reactive group is located at an internal position of the primer e.g., the primer contains one or more modified nucleotides, such as aminoallyl deoxyuridine 5′-triphosphate (dUTP) nucleotide(s). In embodiments, the immobilized oligonucleotide primers may be used to aid in tethering the extension product to a confined area and may not be extended. In embodiments, the immobilized oligonucleotide primers may be used to aid in tethering the extension product to a confined area and may also be capable of being extended. For example, one or more immobilized oligonucleotides may be used to aid in tethering the extension product to a localized area and may be extended in an exponential RCA amplification reaction.

An alternate method is to start with a reverse transcription step to convert the RNA to cDNA. The cDNA would then act as the target for the padlock probe. In embodiments, the cDNA could serve as a splint for ligation. Yet another method would be to simply bind a large probe directly to the RNA, without doing ligation or RCA. The large probe (e.g., branched DNA or a long concatemer) would carry multiple sites for binding sequencing primers & reading identical barcodes. The drawbacks to this method include higher non-specific background and less efficient binding kinetics of a large probe. An alternate method is to start with RNA and hybridize the padlock probes directly to the RNA.

Probes can be designed to target multiple regions on the RNA of interest. This could be done to enhance the signal, and/or to provide a level of redundancy of targeting in case of mutations in a particular region. The probes that target 2 or more regions of the same RNA transcript could carry identical barcodes (for redundancy), or could carry distinct barcodes (to independently identify the region that is being detected).

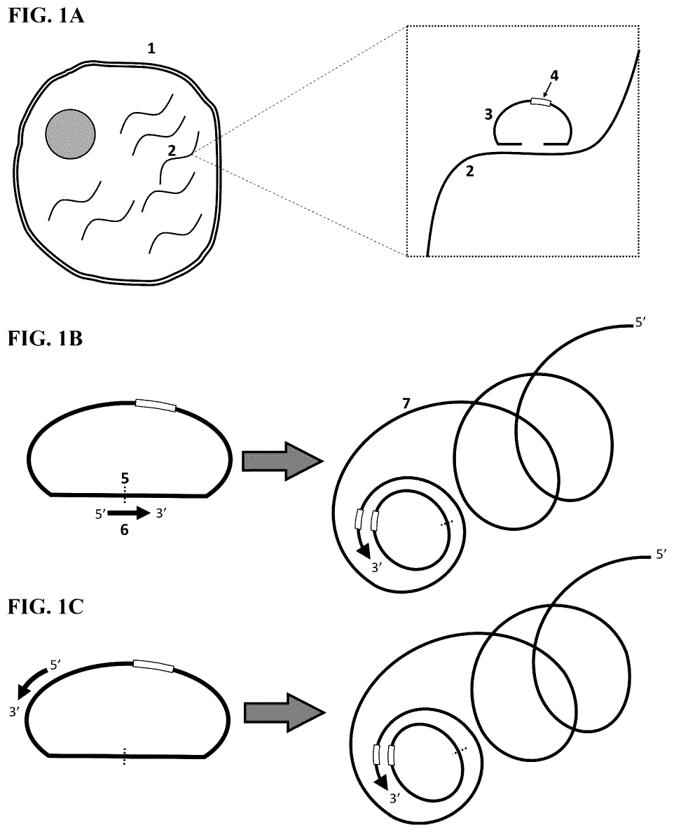

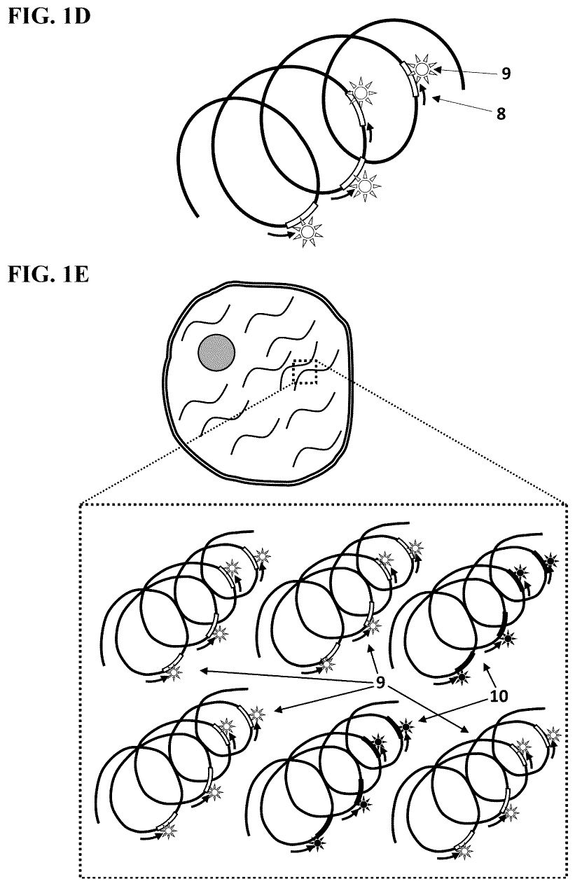

Imaging. To read out the barcode-containing circular oligonucleotides, a sequencing primer is introduced, and the barcode is read out (see, e.g., FIGS. 1D-1E). Preferably, the readout is done by SBS (Sequencing-By-Synthesis), with labeled and reversibly terminated nucleotides. Similar to the amplification primer sequence, and the immobilized oligonucleotide primer described supra, the sequencing primer sequence may contain one or more nucleotides containing functional moieties (e.g., bioconjugate reactive groups) that serve as attachment points to the cell or the matrix in which the cell is embedded (e.g., a hydrogel) or to a cellular component, and the SBS reactions are performed with labeled and reversibly terminated nucleotides. In some embodiments, the sequencing primer binding sequence is the first or second hybridization sequence of the circularizable oligonucleotide. In embodiments, the modified sequencing primer is provided to the matrix in which the cell is embedded following amplification or concurrently with the SBS mixture. The attachment of the SBS product to the matrix via the sequencing primer can help confine and fix the amplicon to a small, localized volume.

Because the identity of all the barcodes is known a priori, the resulting signal can be deconstructed (demultiplexed) into the constituent components (see, e.g., FIG. 2). Each sequencing cycle produces information about the magnitude of the signal in all 4 channels (a subset of 3 or 2 could also be used). The magnitude of the signal in all 4 channels can, for example, be represented by a signal matrix as:
Cycle 1: C₁, T₁, G₁, A₁
Cycle 2: C₂, T₂, G₂, A₂
Cycle 3: C₃, T₃, G₃, A₃
Cycle 4: C₄, T₄, G₄, A₄
Cycle 5: C₅, T₅, G₅, A₅
Cycle 6: C₆, T₆, G₆, A₆

Each barcode can also be represented by a similar matrix. For example, a 6-base barcode such as GTCATA could be represented as:
0, 0, 1, 0
0, 1, 0, 0
1, 0, 0, 0
0, 0, 0, 1
0, 1, 0, 0
0, 0, 0, 1

The signal is then fit to a linear combination of component barcodes. For example, in embodiments when using 4-color detection (i.e., one color per nucleotide) a set of 10 sequencing cycles provides information in 40 dimensions (4 channels per cycle×10 cycles). Any of the up to 410 possible barcodes would point to a unique position in this 40-dimensional space. Linear combinations of barcodes are thus easily resolvable, limited only by the accuracy of the sequencing signals. A typical example might be a set of 1,000-10,000 RNA targets, each encoded by a barcode selected from 4N combinations, where N is the number of sequencing cycles or “digits” in the barcode. With 10 cycles, up to 410, or approximately one million barcodes are available. This allows for the ability to select barcodes that are as far apart as possible in the available space (maximizing the Hamming distance), for more robust demultiplexing.

As illustrated in FIG. 2, each barcode can also be assigned a color (e.g., colors that are visually distinguishable) for the purpose of visualizing the spatial location of the barcode in a cell.

Each sequencing cycle produces varying signal intensities in a voxel for each of the 4 channels. The brightness of the representative pixel corresponds to the local concentration of the barcode and is proportional to the product of the signal and barcode matrices (e.g., the product of the signal and barcode matrices results in a value of 4 for Barcode-1 in FIG. 2, which can also be assigned a color such as green). Subsequent barcodes would also be assigned colors with pixel intensities proportional to the sequencing signal (e.g., the product of the signal and barcode matrices results in a value of 2 for Barcode-2 in FIG. 2, which can also be assigned a color such as red).

Practical limitations, such as noise in the sequencing signal, will limit the total number of RNA transcripts that can be accurately detected in a single resolved volume (voxel). Reasonable upper limits might be 3, 10, 30, or >100 targets per voxel, depending on the performance of the sequencing system.

Imaging. Either 2D or 3D fluorescent imaging modalities can be used. An advantage of 3D imaging is that a larger number of individual volumes can be resolved. 3D fluorescent imaging methods include confocal microscopy, light sheet microscopy, and multi-photon microscopy. For example, if the imaging system has a lateral resolution of 0.5 μm, and a depth resolution of 1.0 μm, a 10×10×10 μm volume would contain 20×20×10=4,000 voxels. If each voxel can resolve 10 barcodes, then this would correspond to a capacity of 40,000 reads in a 10-um cube without pushing the limits of optical resolution.

Further information can be gained by including expansion microscopy if subcellular resolution is required beyond the limits of diffraction, or if an even larger number of reads is desired.

The described methods can be applied to single cells affixed to a transparent substrate, as well as to sections of tissue on a similar substrate. In both cases (individual cells or cells in tissue), the cells are fixed and permeabilized for delivering probes, enzymes, nucleotides and other components required in the reactions.

What is claimed is:

1. A method of generating an amplification product, said method comprising:
a) contacting a target polynucleotide in a cell or tissue with a first oligonucleotide comprising a first target hybridization sequence, wherein said first oligonucleotide comprises a first probe hybridization sequence, a barcode sequence, and a second probe hybridization sequence, and hybridizing the first target hybridization sequence to a first target sequence of said target polynucleotide;
b) contacting the target polynucleotide with a second oligonucleotide comprising a second target hybridization sequence, wherein said second oligonucleotide comprises a third probe hybridization sequence and a fourth probe hybridization sequence, and hybridizing the second target hybridization sequence to a second target sequence of said target polynucleotide;
c) hybridizing a first oligonucleotide probe to said first oligonucleotide and to said second oligonucleotide, wherein said first oligonucleotide probe comprises a sequence complementary to said second probe hybridization sequence and a sequence complementary to said fourth probe hybridization sequence;
d) hybridizing a second oligonucleotide probe to said first oligonucleotide and said second oligonucleotide, wherein said second oligonucleotide probe comprises a sequence complementary to said first probe hybridization sequence and a sequence complementary to said third probe hybridization sequence;
e) extending the second oligonucleotide probe along the barcode sequence with a polymerase to generate a complementary barcode sequence, ligating the complementary barcode sequence to the first oligonucleotide probe, and ligating the first oligonucleotide probe to the second oligonucleotide probe, thereby generating a circular oligonucleotide; and
f) amplifying the circular oligonucleotide, thereby generating an amplification product comprising multiple complements of the complementary barcode sequence.

2. The method of claim 1, further comprising binding a plurality of oligonucleotides to the target polynucleotide and repeating steps c), d), e) and f) for each bound oligonucleotide.

3. The method of claim 1, further comprising detecting the amplification product of step (f).

4. The method of claim 3, wherein detecting the amplification product comprises hybridizing an oligonucleotide associated with a detectable label to the amplification product and identifying said detectable label.

4. The method of claim 1, further comprising sequencing the amplification product of step (f).

6. The method of claim 5, wherein sequencing comprises sequencing by synthesis, sequencing by binding, sequencing by ligation, or pyrosequencing.

7. The method of claim 5, wherein sequencing comprises extending a sequencing primer by incorporating a labeled nucleotide or labeled nucleotide analogue, and detecting the label to generate a signal for each incorporated nucleotide or nucleotide analogue, wherein the sequencing primer is hybridized to the extension product.

5. The method of claim 1, wherein steps (a)-(f) of the method are performed in a cell.

9. The method of claim 8, wherein the cell is permeabilized and immobilized to a solid support surface.

6. The method of claim 1, wherein said second oligonucleotide does not comprise a barcode sequence.

7. The method of claim 1, wherein said first oligonucleotide probe and second oligonucleotide probe do not comprise a barcode sequence.

8. The method of claim 1, wherein the first target sequence is 5′ to the second target sequence of said target polynucleotide.

9. The method of claim 1, wherein amplifying the circular oligonucleotide comprises incubating the circular oligonucleotide with the strand-displacing polymerase (a) for about 1 minute to about 2 hours, and/or (b) at a temperature of about 20° C. to about 50° C.

10. The method of claim 1, wherein the first oligonucleotide, the second oligonucleotide, or both the first and second oligonucleotides comprise one or more locked nucleic acid (LNA) nucleotides.

11. The method of claim 1, wherein the first oligonucleotide probe, the second oligonucleotide probe, or both the first and second oligonucleotide probes comprise one or more locked nucleic acid (LNA) nucleotides.

12. The method of claim 1, wherein the circular oligonucleotide is about 100 to about 1000 nucleotides in length.

13. The method of claim 1, wherein each target hybridization sequence is about 5 to about 35 nucleotides in length.

14. The method of claim 1, wherein the barcode sequence is at least 5, 6, 7, 8, 9, 10, 11, 12, 13, 14, or 15 nucleotides in length.

15. The method of claim 1, wherein the barcode sequence is selected from a known set of barcode sequences.

20. The method of claim 19, wherein each of the known set of barcode sequences is associated with a target hybridization sequence from a known set of target hybridization sequences.
